(19) United States
(12) Patent Application Publication
(10) Pub. No.: US 2004/0180891 A1
(43) Pub. Date: Sep. 16, 2004

(54) Pyrazole derivatives useful in the treatment of hyper-proliferative disorders

(76) Inventors: Uday Khire, Hamden, CT (US); Chengzhi Zhang, Orange, CT (US); Harold C E Kluender, Trumbull, CT (US); Ingo Mugge, New Haven, CT (US); Zhengqui Hong, Milford, CT (US); Jianxing Shao, Cheshire, CT (US); Neil Bifulco, Durham, NC (US); Pamela A Trail, Madison, CT (US); Jacques Dumas, Bethany, CT (US); Rico C Lavoie, Hamden, CT (US); Xaio-Gao Liu, New Haven, CT (US); Veena Agarwal, Milford, CT (US); Sharad K Verma, New Haven, CT (US); Lei Wang, New Haven, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/489,796

(22) PCT Filed: Sep. 20, 2002
(86) PCT No.: PCT/US02/29958

Related U.S. Application Data
(60) Provisional application No. 60/324,573, filed on Sep. 25, 2001.

(51) Int. Cl. A61K 31/5377; A61K 31/4439; A61K 31/4152; C07D 413/02; C 07D 4 3/02

(52) U.S. Cl. 514/235.5; 514/341; 514/406; 544/140; 546/276.4; 548/366.1

(57) ABSTRACT

This invention relates to pyrazole derivatives of

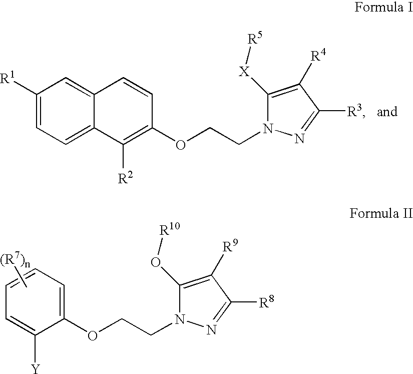

that are useful for treating hyper-proliferative disorders and angiogenesis dependent disorders.

### FIELD OF THE INVENTION

This invention relates to novel pyrazole compounds, pharmaceutical compositions containing such compounds and the use of those compounds and compositions for the prevention and/or treatment of hyper-proliferative disorders and diseases associated with deregulated angiogenesis.

### DESCRIPTION OF THE INVENTION

Compounds of the Present Invention

One embodiment of the present invention relates to a compound of the formula

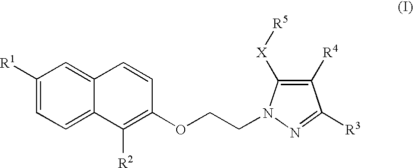

wherein

R¹ is H, halo or CN;

R² is H, CN, COR⁶, halo, or C₁-C₆alkyl;

R³is CF₃,

C₁-C₆alkyl substituted with 0-1 substituent selected from

phenyl where the phenyl group is substituted with 0-5 substituents selected from C₁-C₆alkyl, C₁-C₆alkoxy, CF₃, NO₂, halo, CONH₂ and COOR⁶, and

phenoxy where the phenoxy group is substituted with 0-5 substituents selected from C₁-C₆alkyl, C₁-C₆alkoxy, CF₃, NO₂, halo, CONH₂ and COOR⁶, or

phenyl substituted with 0-5 substituents selected from C₁-C₆alkyl, C₁-C₆alkoxy, CF₃, NO₂, halo, CONH₂ and COOR⁶,

furyl substituted with 0-2 substituents selected from C₁-C₆alkyl and CF₃,

thienyl substituted with 0-2 substituents selected from halo and C₁-C₆alkoxy,

isoxazolyl substituted with 0-2 C₁-C₆alkyl substituents,

pyridyl, or

benzodioxole;

R⁴ is H, C₁-C₆alkyl, halo or cyano;

X is 0 or NH;

R⁵ is C₁-C₆alkyl substituted with 0-1 substituent selected from CF₃, pyridyl, morpholinyl, and thienyl substituted with 0-1 C₁-C₆alkyl group;

R⁶ is H or C₁-C₆alkyl;

or a pharmaceutically acceptable salt thereof.

The terms identified above have the following meaning throughout:

C₁-C₆ alkyl means straight or branched chain alkyl groups having from one to about six saturated carbon atoms which may be linear or branched with single or multiple branching. Such groups include methyl, ethyl, n-propyl, isopropyl, n-butyl, isobutyl, sec-butyl, tert-butyl, and the like.

The term C₁-C₆ alkoxy means straight or branched chain alkoxy groups having from one to about six saturated carbon atoms which may be linear or branched with single or multiple branching, and includes such groups as methoxy, ethoxy, n-propoxy, isopropoxy, n-butoxy, isobutoxy, sec-butoxy, tert-butoxy, and the like.

Halo means fluoro, chloro or bromo. Fluoro and bromo are preferred, and fluoro is most preferred for R⁴, whereas Br is most preferred for R¹ and R².

When any moiety is “substituted”, from none to up to the highest number of substituents indicated can be attached to that moiety. Each substituent can be located at any available carbon atom on the moiety and can be attached through any available atom on the substituent. “Any available atom” means any atom at any position on the moiety that is chemically accessible through means known in the art or taught herein and that does not create an unduly unstable molecule. When there are two or more substituents on any moiety, each substituent is defined independently of any other substituent and can, accordingly, be the same or different.

The compounds of Formula I may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of Formula I which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

The use of pharmaceutically acceptable salts of the compounds of Formula I are also within the scope of this invention. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate. The term acid addition alts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Whenever used herein, the term “compounds of this invention”, “Formula I compounds”, “Formula II compounds”, and the like, are intended to include also the pharmaceutically acceptable salts and all stereoisomeric forms of the referenced

Illustrative examples of the compounds of Formula I include those compounds described below in Table I.

TABLE 1

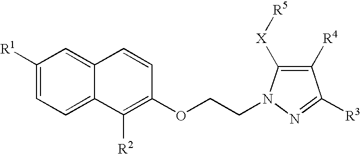

| Example | | | | | | |
|---|---|---|---|---|---|---|
| No. | R¹ | R² | R³ | R⁴ | X | R⁵ |
| 1 | Br | Br | 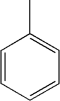 | H | O | Et |
| 2 | Br | Br | Me | H | O | Et |
| 3 | Br | Br | 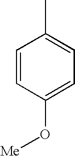 | H | O | Et |
| 4 | Br | Br | 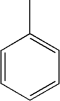 | H | O | Me |
| 5 | Br | Br | 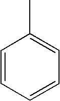 | H | O | i-Pr |
| 6 | Br | Br | 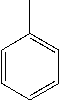 | H | O | n-Pr |
| 7 | Br | H | 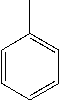 | H | O | Et |
| 8 | Br | H | 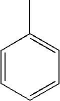 | H | O | Me |
| 9 | Br | H | 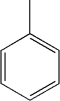 | H | O | i-Pr |
| 10 | Br | H | 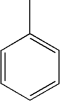 | H | O | n-Pr |
| 11 | Br | Br | 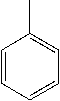 | H | O | n-Bu |
| 12 | Br | Br | 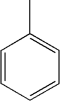 | H | O | n-Pent |
| 13 | Br | H | CF₃ | H | O | Et |
| 14 | Br | H | CF₃ | H | O | n-Pr |
| 15 | Br | H | CF₃ | H | O | n-Bu |
| 16 | Br | H | CF₃ | H | O | n-Pent |
| 17 | Br | H | CF₃ | H | O | Et |
| 18 | Br | H | 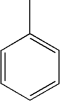 | H | O | 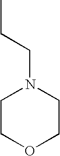 |
| 19 | Br | H | 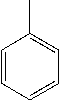 | H | O | 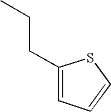 |
| 20 | Br | H | 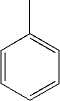 | H | O | 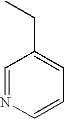 |
| 21 | H | Br | 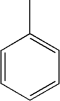 | H | O | Et |
| 22 | Br | Br | 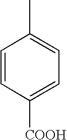 | H | O | Et |
| 23 | Br | Br | 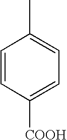 | H | O | n-Pr |
| 24 | H | 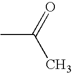 | 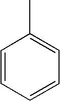 | H | O | Et |
| 25 | Br | Br | 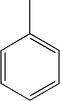 | Me | O | Et |
| 26 | Br | Br | 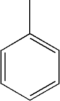 | F | O | Et |
| 27 | Br | Br | 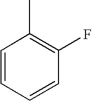 | H | O | Et |
| 28 | Br | Br | 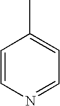 | H | O | Et |
| 29 | Br | Br | 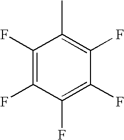 | H | O | Et |
| 30 | Br | Br | 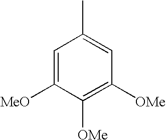 | H | O | Et |
| 31 | Br | Br | 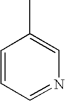 | H | O | Et |
| 32 | Br | Br | 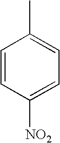 | H | O | Et |
| 33 | Br | Br | 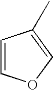 | H | O | Et |
| 34 | Br | Br | 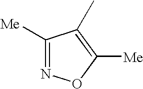 | H | O | Et |
| 35 | Br | Br | 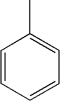 | H | N | (CH₂)₂CF₃ |
| 36 | Br | Br | 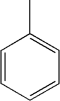 | H | N | Et |
| 37 | Br | Br | 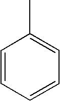 | H | O | CH₂CF₃ |
| 38 | Br | Br | 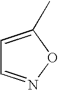 | H | O | Et |
| 39 | Br | Br | 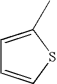 | H | O | Et |
| 40 | Br | Br | 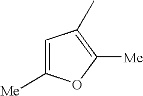 | H | O | Et |
| 41 | Br | Br | 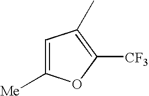 | H | O | Et |
| 42 | Br | Br | 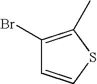 | H | O | Et |
| 43 | Br | Br | 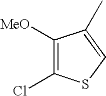 | H | O | Et |
| 44 | Br | Br | 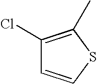 | H | O | Et |
| 45 | Br | Br | 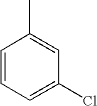 | H | O | Et |
| 46 | Br | Br | 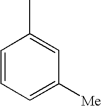 | H | O | Et |
| 47 | Br | Br | 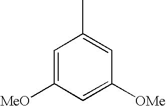 | H | O | Et |
| 48 | Br | Br | 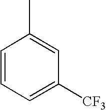 | H | O | Et |
| 49 | Br | Br | 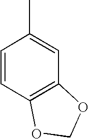 | H | O | Et |
| 50 | Br | Br | 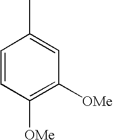 | H | O | Et |
| 51 | Br | Br | 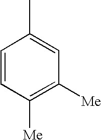 | H | O | Et |
| 52 | Br | Br | 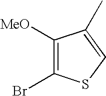 | H | O | Et |
| 53 | Br | Br | 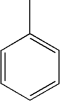 | Cl | O | Et |
| 54 | Br | Br | 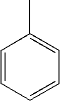 | Br | O | Et |
| 55 | Br | Br | 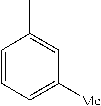 | F | O | Et |
| 56 | Br | Br | 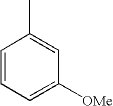 | F | O | Et |
| 57 | Br | Br | 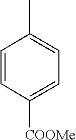 | Br | O | Et |
| 58 | Br | Br | 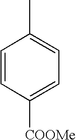 | F | O | Et |
| 59 | Br | Br | 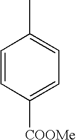 | F | O | n-Pr |
| 60 | Br | Br | 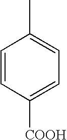 | Br | O | Et |
| 61 | Br | Br | 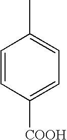 | Br | O | n-Pr |
| 62 | Br | Br | 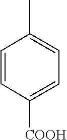 | F | O | Et |
| 63 | Br | Br | 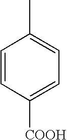 | F | O | n-Pr |
| 64 | Br | Br | 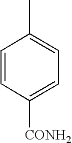 | F | O | Et |
| 65 | Br | Br | 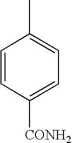 | F | O | n-Pr |
| 66 | Br | Br | 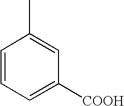 | H | O | Et |
| 67 | Br | Br | 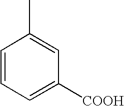 | H | O | n-Pr |
| 68 | Br | Br | 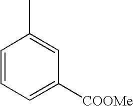 | H | O | Et |
| 69 | Br | Br | 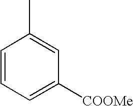 | H | O | n-Pr |
| 70 | Br | Cl | 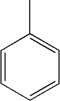 | F | O | Et |
| 71 | Br | Cl | 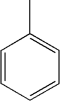 | Br | O | Et |
| 72 | Br | Br | 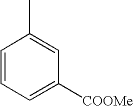 | F | O | Et |
| 73 | Br | Br | 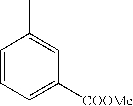 | F | O | n-Pr |
| 74 | Br | Br | 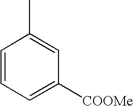 | Br | O | n-Pr |
| 75 | Br | Br | 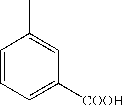 | F | O | Et |
| 76 | Br | Br | 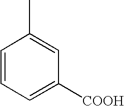 | F | O | n-Pr |
| 77 | Br | Br | 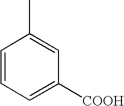 | Br | O | n-Pr |
| 78 | Br | Br | 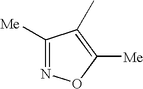 | H | O | Et |
| 79 | Br | Br | 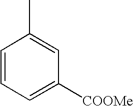 | Br | O | Et |
| 80 | Br | Br | 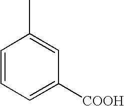 | Br | O | Et |
| 81 | Br | Br | 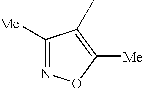 | F | O | Et |
| 82 | Br | Br | 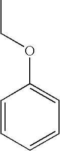 | H | O | Et |
| 83 | CN | CN | 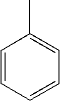 | H | O | Et |
| 84 | CN | Cl | 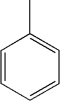 | H | O | Et |
| 85 | Br | Br | 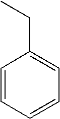 | H | O | Et |
| 86 | Br | Br | 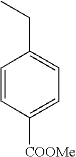 | H | O | Et |
| 87 | Br | Br | 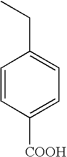 | H | O | Et |

The compound structures of Table 1 correspond to the IUPAC compound names and characterization data below.

1. 1-{2-[(1,6-dibromo-2-naphthyl)oxy]ethyl}-5-ethoxy-3-phenyl-1H-pyrazole: ¹H-NMR (CD₃OD, 400 MHz) δ1.29 (m, 3H), 4.11 (m, 2H), 4.42 (m, 2H), 4.60 (m, 2H), 5.96 (s, 1H), 7.28 (m, 1H), 7.36 (m, 3H), 7.62 (m, 3H), 7.73 (m, 3H), 7.99 (d, J=2.0 Hz, 1H), 8.03 (d, J=9.2 Hz, 1H). LC/MS [M+1]⁺: m/z 517.04.

2. 1-{2-[(1,6-dibromo-2-naphthyl)oxy]ethyl}-5-ethoxy-3-methyl-1H-pyrazole: LC/MS [M]⁺: m/z 454.1, RT 4.65.

3. 1-{2-[(1,6-dibromo-2-naphthyl)oxy]ethyl}-5-ethoxy-3-(4-methoxyphenyl)-1H-pyrazole: LC/MS [M+1]⁺: m/z 547.1, RT 4.53.

4. 1-{2-[(1,6-dibromo-2-naphthyl)oxy]ethyl}-5-methoxy-3-phenyl-1H-pyrazole: ¹H-NMR (CDCl₃, 400 MHz) δ 3.20 (s, 3H), 4.40 (m, 3H), 4.50 (m, 2H), 5.70 (s, 1H), 7.10 (m, 1H), 7.25 (m, 1H), 7.35 (m, 2H), 7.55 (m, 2H), 7.65 (m, 2H), 7.80 (s, 1H), 7.95 (d, 1H). LC/MS [M+1]⁺: m/z 503, RT 3.89.

5. 1-{2-[(1,6-dibromo-2-naphthyl)oxy]ethyl}-5-isopropoxy-3-phenyl-1H-pyrazole: ¹H-NMR (CDCl₃, 400 MHz) δ 2.25 (d, 6H), 4.40 (m, 3H), 4.50 (m, 2H), 5.70 (s, 1H), 7.10 (m, 1H), 7.25 (m, 1H), 7.35 (m, 2H), 7.55 (m, 2H), 7.65 (m, 2H), 7.80 (s, 1H), 7.95 (d, 1H). LC/MS [M+1]⁺: m/z 531, RT 4.37.

6. 1-{2-[(1,6-dibromo-2-naphthyl)oxy]ethyl}-3-phenyl-5-propoxy-1H-pyrazole: ¹H-NMR (CDCl₃, 400 MHz) δ 0.90 (m, 3H), 1.65 (m, 2H), 3.90 (m, 2H), 4.40 (m, 2H), 4.55 (m, 2H), 5.70 (s, 1H), 7.10 (m, 1H), 7.25 (m, 1H), 7.35 (m, 2H), 7.55 (m, 2H), 7.65 (m, 2H), 7.80 (s, 1H), 7.95 (d, 1H). LC/MS [M+1]⁺: m/z 531, RT 4.27.

7. 1-{2-[(6-bromo-2-naphthyl)oxy]ethyl}-5-ethoxy-3-phenyl-1H-pyrazole: TLC (EtOAc-hexanes, 1:1, Rf=0.80); ¹H NMR (CDCl₃): δ 1.45 (t, 3H), 4.17 (q, 2H), 4.45 (t, 4H), 5.8 (s, 1H), 7.1 (s, 1H), 7.16 (d, 1H), 7.3 (d, 1H), 7.4 (t, 2H), 7.45 (d, 1H), 7.5 (d, 1H), 7.67 (d, 1H), 7.8 (d, 2H), 7.9 (s, 1H); HPLC/MS (M)⁺ m/z 437 and (M+2)⁺ m/z 439.

8. 1-{2-[(6-bromo-2-naphthyl)oxy]ethyl}-5-methoxy-3-phenyl-1H-pyrazole: TLC (EtOAc-hexanes 1:3, Rf=0.38,). ¹H NMR (CDCl₃): δ 3.95 (s, 3H), 4.5 (m, 4H), 5.85 (s, 1H), 7.1 (s, 1H), 7.15 (d, 1H), 7.35 (t, 1H), 7.4 (m, 2H), 7.5 (d, 1H), 7.55 (d, 1H), 7.62 (d, 1H), 7.8 (d, 2H), 7.9 (s, 1H).

9. 1-{2-[(6-bromo-2-naphthyl)oxy]ethyl}-5-isopropoxy-3-phenyl-1H-pyrazole: TLC (Rf=0.5, EtOAc-hexanes 1:3); ¹H NMR (CDCl₃): δ 1.37 (d, 6H ), 4.4 (m, 5H), 5.78 (s, 1H), 7.05 (s, 1H), 7.1 (d, 1H), 7.27 (d, 1H), 7.4 (m, 2H), 7.45 (d, 1H), 7.5 (d, 1H), 7.6 (d, 1H), 7.75 (d, 2H), 7.9 (s, 1H); HPLC/MS (M+H)⁺ m/z 452.

10. 1-{2-[(6-bromo-2-naphthyl)oxy]ethyl}-3-phenyl-5-propoxy-1H-pyrazole:TLC (33% EtOAc/Hexanes, Rf=0.5), LC/MS [M+1]⁺: m/z 452.36, RT 3.29

11. 5-butoxy-1-{2-[(1,6-dibromo-2-naphthyl)oxy]ethyl}-3-phenyl-1H-pyrazole: LC/MS [M+1]⁺: m/z 545, RT 4.47.

12. 1-{2-[(1,6-dibromo-2-naphthyl)oxy]ethyl}-5-(pentyloxy)-3-phenyl-1H-pyrazole:LC/MS [M+1]⁺: m/z 559, RT 4.62.

13.1-{2-[(6-bromo-2-naphthyl)oxy]ethyl}-5-ethoxy-3-(trifluoromethyl)-1H-pyrazole: LC/MS [M+1]⁺: m/z 431, RT 3.80.

14. 1-{2-[(6-bromo-2-naphthyl)oxy]ethyl}-5-propoxy-3-(trifluoromethyl)-1H-pyrazole: LC/MS [M+1]⁺: m/z 445, RT 3.99.

15. 1-{2-[(6-bromo-2-naphthyl)oxy]ethyl}-5-butoxy-3-(trifluoromethyl)-1H-pyrazole: LC/MS [M+1]⁺: m/z 459, RT 4.13.

16. 1-{2-[(6-bromo-2-naphthyl)oxy]ethyl}-5-(pentyloxy)-3-(trifluoromethyl)-1H-pyrazole: LC/MS [M+1]⁺: m/z 473, RT 4.22.

17. 1-{2-[(1,6-dibromo-2-naphthyl)oxy]ethyl}-5-ethoxy-3-(trifluoromethyl)-1H-pyrazole: LC/MS [M+1]⁺: m/z 509, RT 4.02.

18. 4-{2-[(1-{2-[(6-bromo-2-naphthyl)oxy]ethyl}-3-phenyl-1H-pyrazol-5-yl)oxy]ethyl}-morpholine: TLC (Rf=0.18 EtOAc); ¹H NMR (CDCl₃): δ 2.55 (m, 4H), 2.78 (t, 2H),), 3.73 (m, 4H), 4.2 (t, 2H), ), 4.45 (m, 4H), 5.85 (s, 1H), 7.05 (s, 1H), 7.1 (d, 1H), 7.3 (m, 1H), ), 7.35 (m, 2H), 7.4 (m, 2H), 7.6 (d, 1H), 7.7 (d, 2H), 7.9 (s, 1H); HPLC/MS (M)⁺ m/z 522.

19. 1-{2-[(6-bromo-2-naphthyl)oxy]ethyl}-3-phenyl-5-[2-(2-thienyl)ethoxy]-1H-pyrazole: TLC showed a single spot; Rf=0.4 (EtOAc-hexanes 1:4); ); ¹H NMR (CDCl₃): δ 3.3 (t, 2H), 4.3 (t, 2H), 4.45 (t, 4H), 5.8 (s, 1H), 6.9 (m, 2H), 7.05 (s, 1H), 7.1 (m, 2H), 7.3 (d, 1H), 7.37 (m, 2H), 7.48 (d, 1H), 7.5 (d, 1H), 7.65 (d, 1H), 7.75 (d, 2H), 7.9 (s, 1H); HPLC/MS (M)⁺ m/z 519 and (M+2)⁺ m/z 521.

20. 3-{[(1-{2-[(6-bromo-2-naphthyl)oxy]ethyl}-3-phenyl-1H-pyrazol-5-yl)oxy]methyl}pyridine: TLC (Rf=0.18, EtOAc); HPLC/MS (M)⁺ m/z 500.

21. 1-{2-[(1-bromo-2-naphthyl)oxy]ethyl}-5-ethoxy-3-phenyl-1H-pyrazole: LC/MS [M]⁺: m/z 437, RT 3.73. ¹H-NMR (CDCl₃, 400 MHz) δ 1.38 (m, 3H), 4.05 (m, 2H), 4.40 (m, 2H), 4.50 (m, 2H), 5.75 (s, 1H), 7.10 (m, 1H), 7.25 (m, 2H), 7.35 (m, 3H), 7.68 (m, 3H), 8.20 (d, 1H).

22. 4-(1-{2-[(1,6-dibromo-2-naphthyl)oxy]ethyl}-5-ethoxy-1H-pyrazol-3-yl)benzoic acid: LC/MS [M+1]⁺: m/z 561, RT 3.63. ¹H-NMR (DMSO, 400 MHz) δ 1.05 (m, 3H), 3.90 (m, 2H), 4.18 (m, 2H), 4.39 (m, 2H), 6.05 (s, 1H), 7.35 (m, 1H), 7.50 (m, 1H), 7.65 (m, 2H), 7.75 (m, 4H), 8.0 (s, 1H).

23. 4-(1-{2-[(1,6-dibromo-2-naphthyl)oxy]ethyl}-5-propoxy-1H-pyrazol-3-yl)benzoic acid: LC/MS [M+1]⁺: m/z 575, RT 3.77.

24. 1-{2-[2-(5-ethoxy-3-phenyl-1H-pyrazol-1-yl)ethoxy]-1-naphthyl}ethanone: TLC: R_{f}=0.43 (30% EtOAc-hexanes); HPLC/MS (M)⁺ m/z 400, RT 3.25

25. 1-{2-[(1,6-dibromo-2-naphthyl)oxy]ethyl}-5-ethoxy-4-methyl-3-phenyl-1H-pyrazole: ¹H-NMR (CDCl₃, 400 MHz) δ 1.35 (t, 3H), 2.05 (s, 3H), 4.16 (q, 2H), 4.41 (t, 2H), 4.53 (t, 2H), 7.12 (d, 1H), 7.25 (m, 1H), 7.34 (t, 2H), 7.50-7.60 (m, 4H), 7.84 (d, 1H), 7.99 (d, 1H). LC/MS [M+1]⁺: m/z 531.15.

26. 1-{2-[(1,6-dibromo-2-naphthyl)oxy]ethyl}-5-ethoxy-4-fluoro-3-phenyl-1H-pyrazole: ¹H-NMR (CD₃OD, 400 MHz) δ 1.34 (t, 3H), 4.35-4.44 (m, 4H), 4.61 (t, 2H). 7.30-7.43 (m, 4H), 7.63 (dd, 1H), 7.72-7.80 (m, 3H), 8.01-8.08 (m, 2H). LC/MS [M+1]⁺: m/z 535.04. Corresponding β-keto ester was prepared according to the literature procedure (Kim, D. Y.; Oh, D. Y. Tetrahedron letters 1996, 37, 653-654)

27. 1-{2-[(1,6-dibromo-2-naphthyl)oxy]ethyl}-5-ethoxy-3-(2-fluorophenyl)-1H-pyrazole: LC/MS [M+1]⁺: m/z 535, RT 4.14

28. 4-(1-{2-[(1,6-dibromo-2-naphthyl)oxy]ethyl}-5-ethoxy-1H-pyrazol-3-yl)pyridine: HPLC-MS m/z 518, RT 2.89

29. 1-{2-[(1,6-dibromo-2-naphthyl)oxy]ethyl}-5-ethoxy-3-(2,3,4,5,6-pentafluorophenyl)-1H-pyrazole: HPLC-MS m/z 607, RT 4.32

30. 1-{2-[(1,6-dibromo-2-naphthyl)oxy]ethyl}-5-ethoxy-3-(3,4,5-trimethoxyfluorophenyl)-1H-pyrazole: HPLC-MS m/z 607, RT 3.87

31. 3-(1-{2-[(1,6-dibromo-2-naphthyl)oxy]ethyl}-5-ethoxy-1H-pyrazol-3-yl)pyridine: HPLC-MS m/z 518, RT 3.23

32. 1-{2-[(1,6-dibromo-2-naphthyl)oxy]ethyl}-5-ethoxy-3-(4-nitrophenyl)-1H-pyrazole: LC/MS [M+1]⁺: m/z 535.3, RT 4.38.

33. 1-{2-[(1,6-dibromo-2-naphthyl)oxy]ethyl}-5-ethoxy-3-(3-furyl)-1H-pyrazole: HPLC-MS m/z 507, RT 3.74

34. 4-(1-{2-[(1,6-dibromo-2-naphthyl)oxy]ethyl}-5-ethoxy-1H-pyrazol-3-yl)-3,5-dimethylisoxazole: HPLC-MS m/z 536.3, RT 3.68

35. N-(1-{2-[(1,6-dibromo-2-naphthyl)oxy]ethyl}-3-phenyl-1H-pyrazol-5-yl)-N-(3,3,3-trifluoropropyl)amine: ¹H NMR (CDCl₃) δ 8.05 (1H, d, J=5 Hz), 7.93 (1H, s), 7.76 (2H, dd, J=6, 3 Hz), 7.68 (1H, d, J=6 Hz), 7.65-7.61 (2H, m), 7.40-7.36 (3H, m), 7.17 (1H, d, J=6 Hz), 5.83 (1H, s), 4.5-54.51 (4H, m), 3.48 (2H, q, J=6 Hz), 2.53-2.51 (2H, m). LC/MS (m+1)⁺ m/z 584.3 TLC Rf=0.25 (EtOAc/Hex=2/3)

36. 1-{2-[(1,6-dibromo-2-naphthyl)oxy]ethyl}-N-ethyl-3-phenyl-1H-pyrazol-5-amine: ¹H-NMR (CDCl₃, 400 MHz) δ 1.25 (t, 3H), 3.14 (q, 2H), 4.40-4.62 (m, 4H), 5.75 (s, 1H), 7.05-7.15 (m, 1H), 7.30-7.40 (m, 2H), 7.60-7.78 (m, 4H), 7.90 (s, 1H), 8.00 (d, 1H). LC/MS [M+1]⁺: m/z 516.04.

37. N-(1-{2-[(1,6-dibromo-2-naphthyl)oxy]ethyl}-3-phenyl-1H-pyrazol-5-yl)-N-(2,2,2-trifluoroethyl)amine: ¹H NMR (CDCl₃, δ ppm) 8.05 (1H, d, J=5 Hz), 7.93 (1H, s), 7.76 (2H, dd, J=6, 3 Hz), 7.68 (1H, d, J=6 Hz), 7.65-7.61 (2H, m), 7.40-7.36 (3H, m), 7.17 (1H, d, J=6 Hz), 5.83 (1H, s), 4.72-4.68 (2H, m), 4.62-4.58 (2H, m), 3.82-3.76 (2H, m). LC/MS (m+1)⁺ m/z=570.0, TLC Rf=0.7 (EtOAc/Hex=1/1) 38. 5-(1-{2-[(1,6-dibromo-2-naphthyl)oxy]ethyl}-5-ethoxy-1H-pyrazol-3-yl)isoxazole: HPLC-MS m/z 508.3, RT 3.6

39. 1-{2-[(1,6-dibromo-2-naphthyl)oxy]ethyl}-5-ethoxy-3-(2-thienyl)-1H-pyrazole: HPLC-MS m/z 523.2, RT 3.98

40. 1-{2-[(1,6-dibromo-2-naphthyl)oxy]ethyl}-3-(2,5-dimethyl-3-furyl)-5-ethoxy-1H-pyrazole: HPLC-MS m/z 535.3, RT 4.12

41. 1-{2-[(1,6-dibromo-2-naphthyl)oxy]ethyl}-5-ethoxy-3-[5-methyl-2-(trifluoromethyl)-3-furyl]-1H-pyrazole: HPLC-MS m/z 589.3, RT 4.32

42. 3-(3-bromo-2-thienyl)-1-{2-[(1,6-dibromo-2-naphthyl)oxy]ethyl}-5-ethoxy-1H-pyrazole: HPLC-MS m/z 603.1, RT 4.38

43. 3-(5-chloro-4-methoxy-3-thienyl)-1-{2-[(1,6-dibromo-2-naphthyl)oxy]ethyl}-5-ethoxy-1H-pyrazole: HPLC-MS m/z 587.2, RT 4.36

44. 3-(3-chloro-2-thienyl)-1-{2-[(1,6-dibromo-2-naphthyl)oxy]ethyl}-5-ethoxy-1H-pyrazole: HPLC-MS m/z 557.2, RT 4.26

45. 3-(3-chlorophenyl)-1-{2-[(16-dibromo-2-naphthyl)oxy]ethyl}-5-ethoxy-1H-pyrazole: HPLC-MS m/z 551.3, RT 4.35

46. 1-{2-[(1,6-dibromo-2-naphthyl)oxy]ethyl}-5-ethoxy-3-(3-methylphenyl)-1H-pyrazole: HPLC-MS m/z 531.3, RT 4.16

47. 1-{2-[(1,6-dibromo-2-naphthyl)oxy]ethyl}-3-(3,5-dimethoxyphenyl)-5-ethoxy-1H-pyrazole: HPLC-MS m/z 577.3, RT 3.97

48. 1-{2-[(1,6-dibromo-2-naphthyl)oxy]ethyl}-5-ethoxy-3-[3-(trifluoromethyl)phenyl]-1H-pyrazole: HPLC-MS m/z 585.3, RT 4.38

49. 3-(1,3-benzodioxol-5-yl)-1-{2-[(1,6-dibromo-2-naphthyl)oxy]ethyl}-5-ethoxy-1H-pyrazole: HPLC-MS m/z 561.28, RT 3.94

50. 1-{2-[(1,6-dibromo-2-naphthyl)oxy]ethyl}-3-(3,4-dimethoxyphenyl)-5-ethoxy-H-pyrazole: HPLC-MS m/z 577.3, RT 3.72

51. 1-{2-[(1,6-dibromo-2-naphthyl)oxy]ethyl}-3-(3,4-dimethylphenyl)-5-ethoxy-1H-pyrazole: HPLC-MS m/z 545.3, RT 4.22

52. 3-(5-bromo-4-methoxy-3-thienyl)-1-{2-[(1,6-dibromo-2-naphthyl)oxy]ethyl}-5-ethoxy-1H-pyrazole: HPLC-MS m/z 633.2, RT 4.27

53. 4-chloro-1-{2-[(1,6-dibromo-2-naphthyl)oxy]ethyl}-5-ethoxy-3-phenyl-1H-pyrazole: HPLC-MS m/z 551.3, RT 4.33

54. 4-bromo-1-{2-[(1,6-dibromo-2-naphthyl)oxy]ethyl}-5-ethoxy-3-phenyl-1H-pyrazole: HPLC-MS m/z 597.3, RT 4.38

55. 1-{2-[(1,6-dibromo-2-naphthyl)oxy]ethyl}-5-ethoxy-4-fluoro-3-(3-methylphenyl)-1H-pyrazole: HPLC-MS m/z (MH+) 547.6, 549.3, RT 4.60 min

56. 1-{2-[(1,6-dibromo-2-naphthyl)oxy]ethyl}-5-ethoxy-4-fluoro-3-(3-methoxyphenyl)-1H-pyrazole: HPLC-MS m/z (MH+) 563.7, 565.2, RT 4.40 min

57. methyl 4-(4-bromo-1-{2-[(1,6-dibromo-2-naphthyl)oxy]ethyl}-5-ethoxy-1H-pyrazol-3-yl)benzoate: HPLC-MS m/z 655, RT 4.55

58. methyl 4-(1-{2-[(1,6-dibromo-2-naphthyl)oxy]ethyl}-5-ethoxy-4-fluoro-1H-pyrazol-3-yl)benzoate: HPLC-MS m/z 593.1, RT 4.49

59. methyl 4-(1-{2-[(1,6-dibromo-2-naphthyl)oxy]ethyl}-4-fluoro-5-propoxy-1H-pyrazol-3-yl)benzoate: HPLC-MS m/z 607.1, RT 4.65

60. 4-(4-bromo-1-{2-[(1,6-dibromo-2-naphthyl)oxy]ethyl}-5-ethoxy-1H-pyrazol-3-yl)benzoic acid: HPLC-MS m/z 639.2, RT 4.02

61. 4-(4-bromo-1-{2-[(1,6-dibromo-2-naphthyl)oxy]ethyl}-5-propoxy-1H-pyrazol-3-yl)benzoic acid: HPLC-MS m/z 653.2, RT 4.03

62. 4-(1-{2-[(1,6-dibromo-2-naphthyl)oxy]ethyl}-5-ethoxy-4-fluoro-1H-pyrazol-3-yl)benzoic acid: HPLC-MS m/z 579.3, RT 3.78

63. 4-(1-{2-[(1,6-dibromo-2-naphthyl)oxy]ethyl}-4-fluoro-5-propoxy-1H-pyrazol-3-yl)benzoic acid: HPLC-MS m/z 593.3, RT 3.85

64. 4-(1-{2-[(1,6-dibromo-2-naphthyl)oxy]ethyl}-5-ethoxy-4-fluoro-1H-pyrazol-3-yl)benzamide: HPLC-MS m/z 578.3, RT 3.39

65. 4-(1-{2-[(1,6-dibromo-2-naphthyl)oxy]ethyl}-4-fluoro-5-propoxy-1H-pyrazol-3-yl)benzamide: HPLC-MS m/z 592.3, RT 3.6

66. 3-(1-{2-[(1,6-dibromo-2-naphthyl)oxy]ethyl}-5-ethoxy-1H-pyrazol-3-yl)benzoic acid: HPLC-MS m/z 559.2, RT 4.2

67. 3-(1-{2-[(1,6-dibromo-2-naphthyl)oxy]ethyl}-5-propoxy-1H-pyrazol-3-yl)benzoic acid: HPLC-MS m/z 573.1, RT 4.37

68. methyl 3-(1-{2-[(1,6-dibromo-2-naphthyl)oxy]ethyl}-5-ethoxy-1H-pyrazol-3-yl)benzoate: HPLC-MS m/z 573.2, RT 4.63

69. methyl 3-(1-{2-[(1,6-dibromo-2-naphthyl)oxy]ethyl}-5-propoxy-1H-pyrazol-3-yl)benzoate: HPLC-MS m/z 587.2, RT 4.78

70. 1-{2-[(6-bromo-1-chloro-2-naphthyl)oxy]ethyl}-5-ethoxy-4-fluoro-3-phenyl-1H-pyrazole: TLC Rf=0.40 (38% yield). LC/MS: MW+1 at m/z 489, RT 4.71 min. ¹H NMR (CD₂Cl₂) δ: 8.1 (m, 1H), 7.95 (d, 1H), 7.8 (m, 2H), 7.7 (m, 2H), 7.4 (m, 4H), 4.6 (t, 2H), 4.5 (t, 2H), 4.4 (2H), 1.4 (t, 3H).

71. 4-bromo-1-{2-[(6-bromo-1-chloro-2-naphthyl)oxy]ethyl}-5-ethoxy-3-phenyl-1H-pyrazole: HPLC-MS [M+1]⁺: m/z 549, RT 5.12

72. methyl 3-(1-{2-[(1,6-dibromo-2-naphthyl)oxy]ethyl}-5-ethoxy-4-fluoro-1H-pyrazol-3-yl)benzoate: HPLC-MS m/z 591.1, RT 4.85

73. methyl 3-(1-{2-[(1,6-dibromo-2-naphthyl)oxy]ethyl}4-fluoro-5-propoxy-1H-pyrazol-3-yl)benzoate: HPLC-MS m/z 605.1, RT 5.00

74. methyl 3-(4-bromo-1-{2-[(1,6-dibromo-2-naphthyl)oxy]ethyl}-5-propoxy-1H-pyrazol-3-yl)benzoate: HPLC-MS m/z 667, RT 5.07

75. 3-(1-{2-[(1,6-dibromo-2-naphthyl)oxy]ethyl}-5-ethoxy-4-fluoro-1H-pyrazol-3-yl )benzoic acid: m/z 579.3, RT 3.82

76. 3-(1-{2-[(1,6-dibromo-2-naphthyl)oxy]ethyl}-4-fluoro-5-propoxy-1H-pyrazol-3-yl) benzoic acid: m/z 593.3, RT 3.86

77. 3-(4-bromo-1-{2-[(1,6-dibromo-2-naphthyl)oxy]ethyl}-5-propoxy-1H-pyrazol-3-yl)benzoic acid: m/z 655.2, RT 3.99

78. 4-(1-{2-[(1,6-dibromo-2-naphthyl)oxy]ethyl}-5-propoxy-1H-pyrazol-3-yl)-3,5-dimethylisoxazole: HPLC-MS m/z 613.3, RT 4.66

79. methyl 3-(4-bromo-1-{2-[(1,6-dibromo-2-naphthyl)oxy]ethyl}-5-ethoxy-1H-pyrazol-3-yl)benzoate: HPLC-MS m/z 653.1, RT 4.90

80. 3-(4-bromo-1-{2-[(1,6-dibromo-2-naphthyl)oxy]ethyl}-5-ethoxy-1H-pyrazol-3-yl)benzoic acid: HPLC-MS m/z 639.1, RT 4.44

81. 4-(1-{2-[(1,6-dibromo-2-naphthyl)oxy]ethyl}-5-ethoxy-4-fluoro-1H-pyrazol-3-yl)-3,5-dimethylisoxazole: HPLC-MS [M+1]⁺ m/z 552, RT 4.57

82.1 -{2-[(1,6-dibromo-2-naphthyl)oxy]ethyl}-5-ethoxy-3-(phenoxymethyl)-1H-pyrazole: LC/MS [M+1]⁺: m/z 547, RT 4.13

83. 2-[2-(5-ethoxy-3-phenyl-1H-pyrazol-1-yl)ethoxy]-1,6-naphthalenedicarbonitrile: HPLC-MS [M+1]⁺: m/z 409, RT 3.37

84. 5-chloro-6-[2-(5-ethoxy-3-phenyl-1H-pyrazol-1-yl)ethoxy]-2-naphthonitrile: HPLC-MS [M+1]⁺: m/z 418, RT 3.65

85. 3-benzyl-1-{2-[(1,6-dibromo-2-naphthyl)oxy]ethyl}-5-ethoxy-1H-pyrazole: LC/MS (M+1)⁺ : m/z 529, RT 4.01

86. methyl 4-[(1-{2-[(1,6-dibromo-2-naphthyl)oxy]ethyl}-5-ethoxy-1H-pyrazol-3-yl)methyl]benzoate: LC/MS (M)⁺: m/z 600, RT 4.06

87. 4-[(1-{2-[(1,6-dibromo-2-naphthyl)oxy]ethyl}-5-ethoxy-1H-pyrazol-3-yl)methyl]benzoic acid: LC/MS (M)⁺: m/z 572, RT 3.44

Another embodiment of the present invention relates to a compound of the formula:

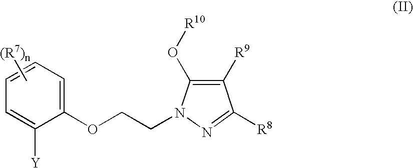

wherein

R⁷ is selected from C₁-C₆alkoxy, Br, Cl, F, CF₃, CN, COOH, NHCOR¹⁴,

C₁-C₆alkyl substituted with 0-1 substituent selected from COOH, NR²R², morpholine, pyrrolidine and piperidine,

phenyl substituted with from 0-3 substituents selected from C₁-C₆ alkyl, C₁-C₆alkoxy, SR¹⁴, Br, Cl, F, CF₃, NH₂ and phenyl,

a C₅-C₆ cyclic group,

thiophene substituted with 0-1 substituent selected from C₁-C₆alkyl and COR¹⁴,

pyridine with 0-2 substituents selected from Br, Cl, F, and C₁-C₆alkyl,

pyrimidine substituted with 0-2 Br atoms,

pyrrole, furan, oxazole, benzothiophene, benzofuran, morpholine, pyrrolidine, piperidine, naphthalene, and benzodioxole;

Y is H, C₁-C₆alkyl, C₁-C₆alkoxy, CN, Br, Cl, F, or I;

R⁸ is phenyl substituted with 0-2 substituents selected from C₁-C₆alkyl, C₁-C₆alkoxy, COR¹¹, and CONH(C₁-C₃alkyl)R¹¹;

R⁹ is H, C₁-C₆alkyl, Br, Cl, and F;

R¹⁰ is C₁-C₆alkyl substituted with 0-1 substituent selected from CF₃, pyridine, morpholine, and thiophene substituted with 0-1 C₁-C₆alkyl group;

R¹¹ is OH, NR¹²R¹², C₁-C₁₀alkyl, C₁-C₆alkoxy, C₁-C₆alkyl substituted with 0-1 substituent selected from CF₃ and morpholine;

R¹² is H and C₁-C₆alkyl;

R¹⁴ is C₁-C₆alkyl;

n is 0, 1, or 2;

or a pharmaceutically acceptable salt thereof.

The terms identified above have the following meaning throughout:

C₁-C₆alkyl and C₁-C₁₀alkyl each means straight or branched chain alkyl groups having from one to about six or about ten saturated carbon atoms respectively, which may be linear or branched with single or multiple branching. Such groups include methyl, ethyl, n-propyl, isopropyl, n-butyl, isobutyl, sec-butyl, tert-butyl, and the like.

The term C₁-C₆alkoxy means straight or branched chain alkoxy groups having from one to about six saturated carbon atoms which may be linear or branched with single or multiple branching, and includes such groups as methoxy, ethoxy, n-propoxy, isopropoxy, n-butoxy, isobutoxy, sec-butoxy, tert-butoxy, and the like.

The C₅-C₆cyclic group is any saturated or unsaturated ring containing 5 or 6 carbon atoms. This group is fused to the phenyl moiety of the core compound at any two adjacent and chemically available carbon atoms. These fused moieties include such groups as naphthalene, tetrahydronaphthalene, indene and indane.

R⁷ is attached to the rest of the molecule of Formula II at the 3, 4, 5 and/or 6 position of the benzyl ring. When n is 2, each R⁷ is selected independently from the other and, accordingly, can be the same or different. When n is 1, R⁷ is preferably attached to the 4 position of the benzyl ring.

When any moiety is “substituted”, it has from none to up to the highest number of substituents indicated. Each substituent can be located at any available carbon or nitrogen atom on the moiety and can be attached through any available atom on the substituent. “Any available atom” means any atom at any position on the moiety that is chemically accessible through means known in the art or taught herein and that does not create an unduly unstable molecule. Specifically, all heterocyclic moieties can be attached to the rest of the molecule through any available carbon atom except pyrrole, which can be attached at any available carbon or nitrogen atom. When there are two or more substituents on any moiety, each substituent is defined independently of any other substituent and can, accordingly, be the same or different.

The compounds of Formula II may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of Formula II which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

The use of pharmaceutically acceptable salts of the compounds of Formula II are also within the scope of this invention. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Illustrative examples of the compounds of Formula II include those compounds described below in Table 2. In Table 2, R¹³ represents the portion of Formula II molecule that corresponds to

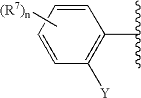

TABLE 2

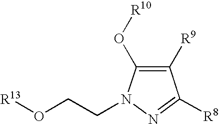

| Ex. | | | | |
|---|---|---|---|---|
| No. | R¹³ | R⁸ | R⁹ | R¹⁰ |
| 88 | 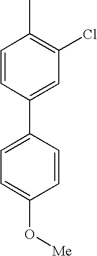 | 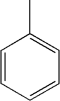 | H | Et |
| 89 | 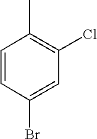 | 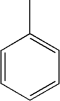 | H | Et |
| 90 | 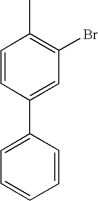 | 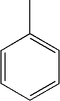 | H | Et |
| 91 | 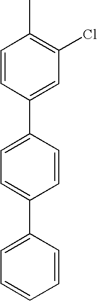 | 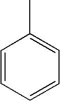 | H | Et |
| 92 | 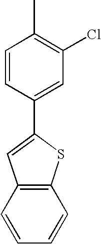 | 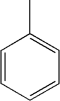 | H | Et |
| 93 | 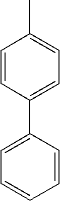 | 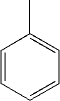 | H | Et |
| 94 | 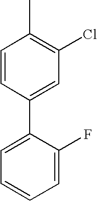 | 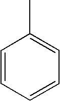 | H | Et |
| 95 | 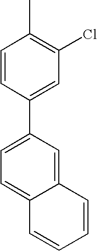 | 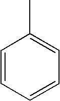 | H | Et |
| 96 | 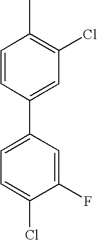 | 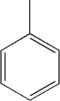 | H | Et |
| 97 | 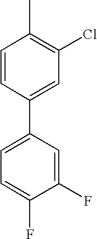 | 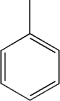 | H | Et |
| 98 | 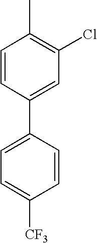 | 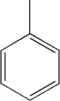 | H | Et |
| 99 | 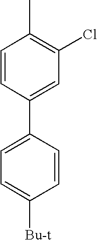 | 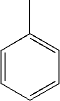 | H | Et |
| 100 | 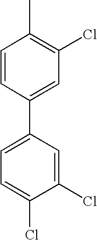 | 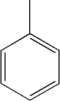 | H | Et |
| 101 | 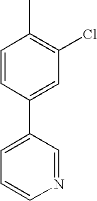 | 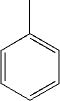 | H | Et |
| 102 | 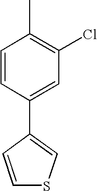 | 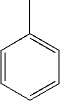 | H | Et |
| 103 | 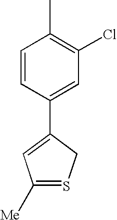 | 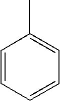 | H | Et |
| 104 | 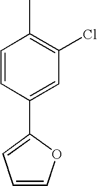 | 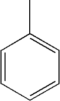 | H | Et |
| 105 | 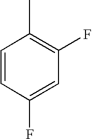 | 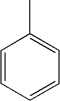 | H | Et |
| 106 | 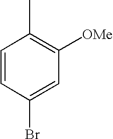 | 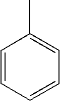 | H | Et |
| 107 | 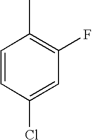 | 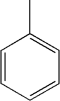 | H | Et |
| 108 | 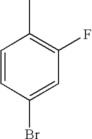 | 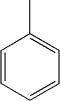 | H | Et |
| 109 | 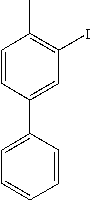 | 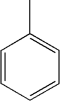 | H | Et |
| 110 | 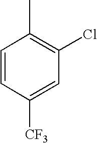 | 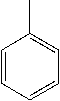 | H | Et |
| 111 | 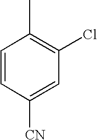 | 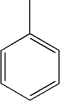 | H | Et |
| 112 | 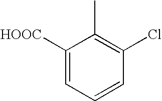 | 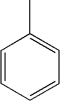 | H | Et |
| 113 | 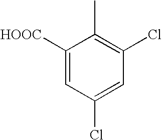 | 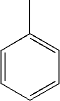 | H | Et |
| 114 | 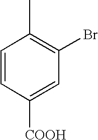 | 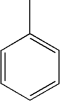 | H | Et |
| 115 | 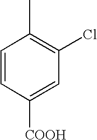 | 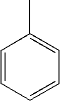 | H | Et |
| 116 | 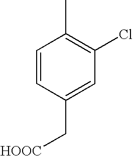 | 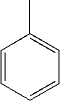 | H | Et |
| 117 | 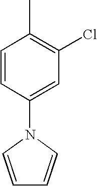 | 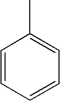 | H | Et |
| 118 | 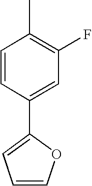 | 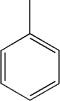 | H | Et |
| 119 | 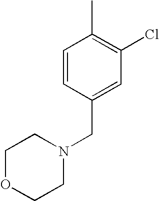 | 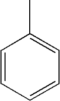 | H | Et |
| 120 | 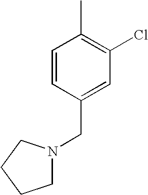 | 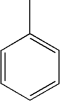 | H | Et |
| 121 | 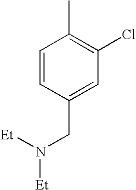 | 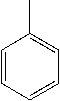 | H | Et |
| 122 | 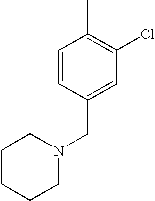 | 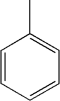 | H | Et |
| 123 | 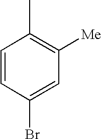 | 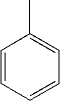 | H | Et |
| 124 | 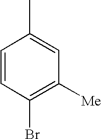 | 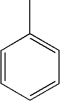 | H | Et |
| 125 | 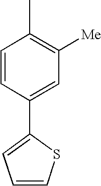 | 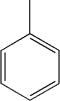 | H | Et |
| 126 | 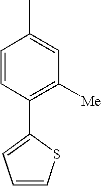 | 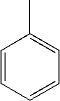 | H | Et |
| 127 | 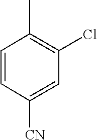 | 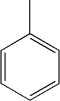 | Me | Et |
| 128 | 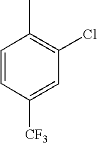 | 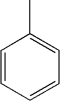 | Me | Et |
| 129 | 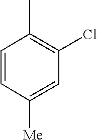 | 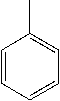 | Me | Et |
| 130 | 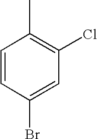 | 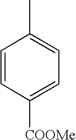 | H | Et |
| 131 | 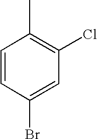 | 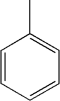 | F | Et |
| 132 | 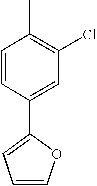 | 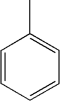 | F | Et |
| 133 | 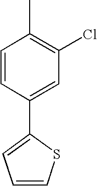 | 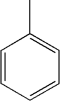 | F | Et |
| 134 | 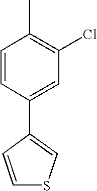 | 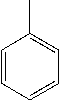 | F | Et |
| 135 | 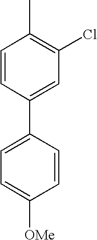 | 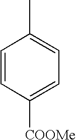 | H | Et |
| 136 | 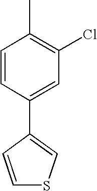 | 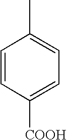 | H | Et |
| 137 | 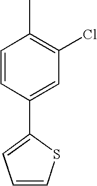 | 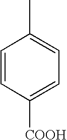 | H | Et |
| 138 | 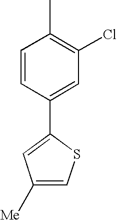 | 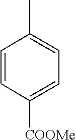 | H | Et |
| 139 | 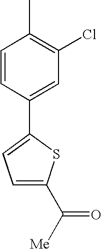 | 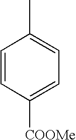 | H | Et |
| 140 | 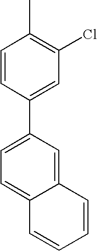 | 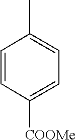 | H | Et |
| 141 | 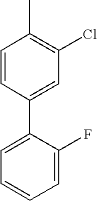 | 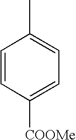 | H | Et |
| 142 | 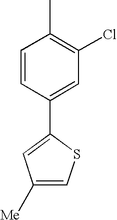 | 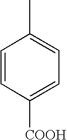 | H | Et |
| 143 | 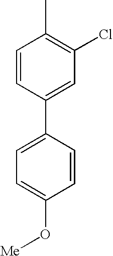 | 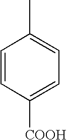 | H | Et |
| 144 | 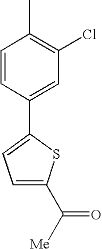 | 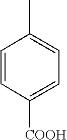 | H | Et |
| 145 | 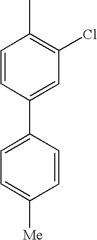 | 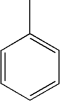 | F | Et |
| 146 | 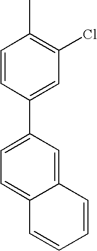 | 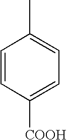 | H | Et |
| 147 | 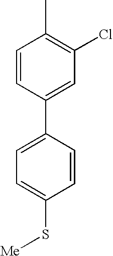 | 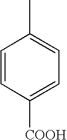 | F | Et |
| 148 | 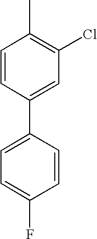 | 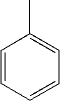 | F | Et |
| 149 | 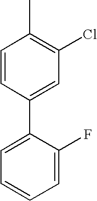 | 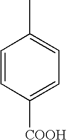 | H | Et |
| 150 | 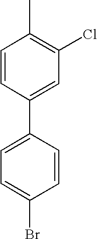 | 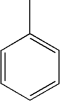 | F | Et |
| 151 | 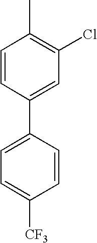 | 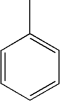 | F | Et |
| 152 | 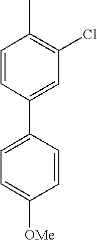 | 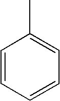 | H | Et |
| 153 | 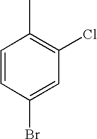 | 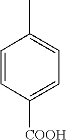 | H | Et |
| 154 | 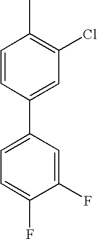 | 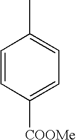 | H | Et |
| 155 | 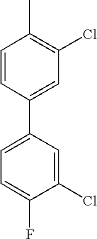 | 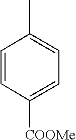 | H | Et |
| 156 | 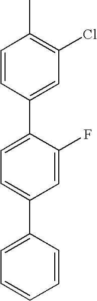 | 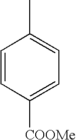 | H | Et |
| 157 | 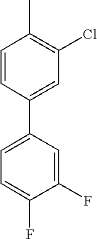 | 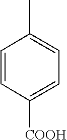 | H | Et |
| 158 | 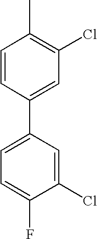 | 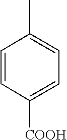 | H | Et |
| 159 | 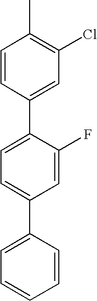 | 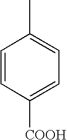 | H | Et |
| 160 | 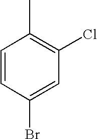 | 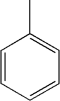 | H | Et |
| 161 | 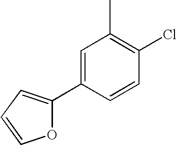 | 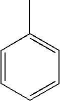 | H | Et |
| 162 | 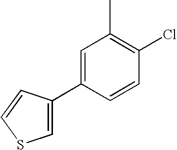 | 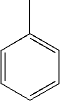 | H | Et |
| 163 | 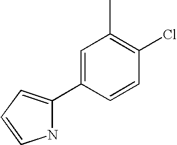 | 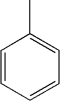 | H | Et |
| 164 | 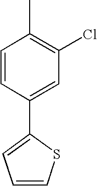 | 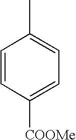 | F | Et |
| 165 | 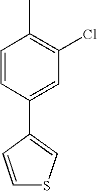 | 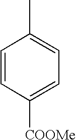 | F | Et |
| 166 | 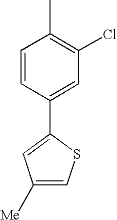 | 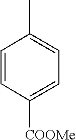 | F | Et |
| 167 | 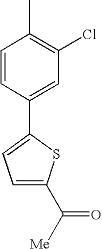 | 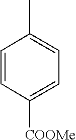 | F | Et |
| 168 | 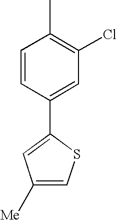 | 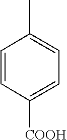 | F | Et |
| 169 | 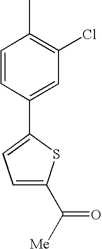 | 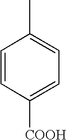 | F | Et |
| 170 | 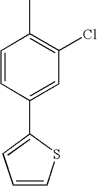 | 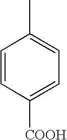 | F | Et |
| 171 | 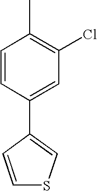 | 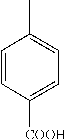 | F | Et |
| 172 | 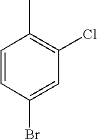 | 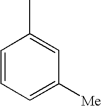 | F | Et |
| 173 | 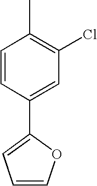 | 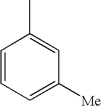 | F | Et |
| 174 | 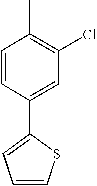 | 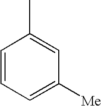 | F | Et |
| 175 | 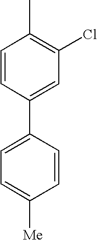 | 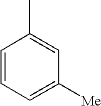 | F | Et |
| 176 | 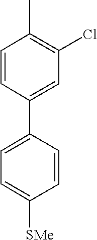 | 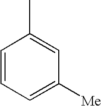 | F | Et |
| 177 | 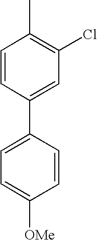 | 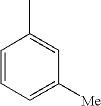 | F | Et |
| 178 | 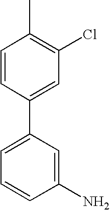 | 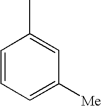 | F | Et |
| 179 | 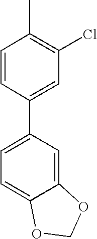 | 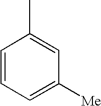 | F | Et |
| 180 | 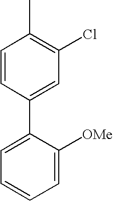 | 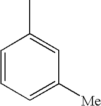 | F | Et |
| 181 | 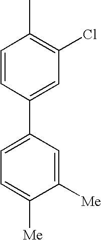 | 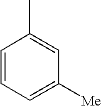 | F | Et |
| 182 | 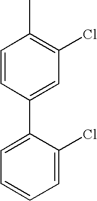 | 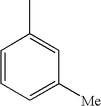 | F | Et |
| 183 | 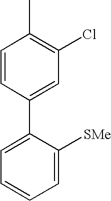 | 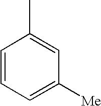 | F | Et |
| 184 | 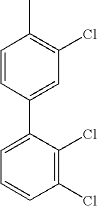 | 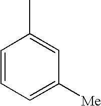 | F | Et |
| 185 | 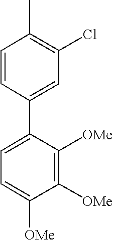 | 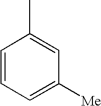 | F | Et |
| 186 | 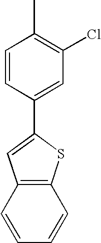 | 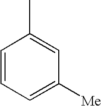 | F | Et |
| 187 | 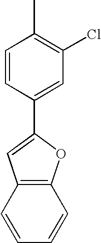 | 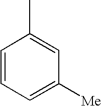 | F | Et |
| 188 | 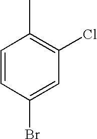 | 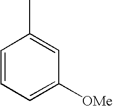 | F | Et |
| 189 | 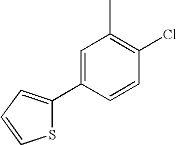 | 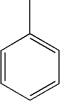 | H | Et |
| 190 | 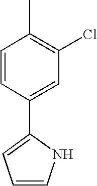 | 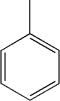 | H | Et |
| 191 | 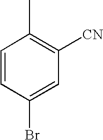 | 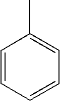 | H | Et |
| 192 | 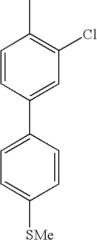 | 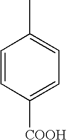 | H | Et |
| 193 | 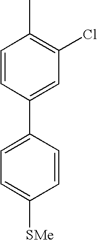 | 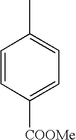 | H | Et |
| 194 | 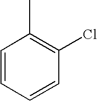 | 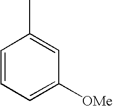 | F | Et |
| 195 | 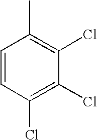 | 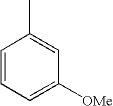 | F | Et |
| 196 | 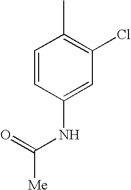 | 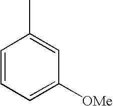 | F | Et |
| 197 | 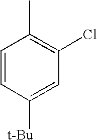 | 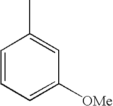 | F | Et |
| 198 | 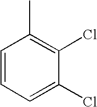 | 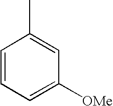 | F | Et |
| 199 | 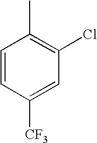 | 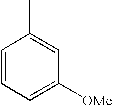 | F | Et |
| 200 | 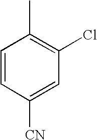 | 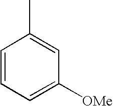 | F | Et |
| 201 | 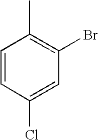 | 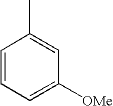 | F | Et |
| 202 | 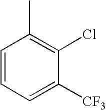 | 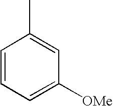 | F | Et |
| 203 | 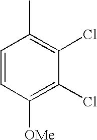 | 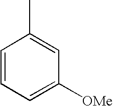 | F | Et |
| 204 | 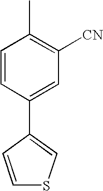 | 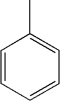 | H | Et |
| 205 | 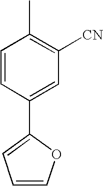 | 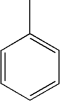 | H | Et |
| 206 | 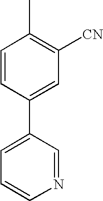 | 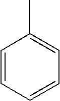 | H | Et |
| 207 | 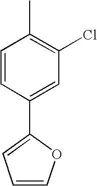 | 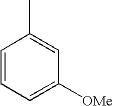 | F | Et |
| 208 | 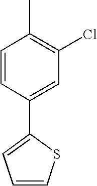 | 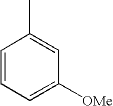 | F | Et |
| 209 | 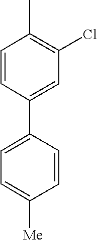 | 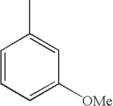 | F | Et |
| 210 | 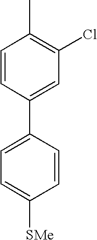 | 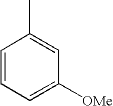 | F | Et |
| 211 | 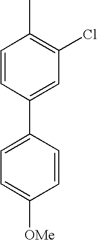 | 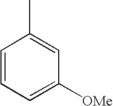 | F | Et |
| 212 | 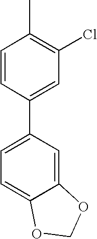 | 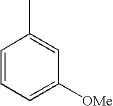 | F | Et |
| 213 | 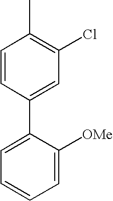 | 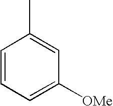 | F | Et |
| 214 | 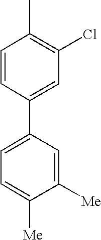 | 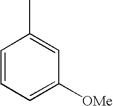 | F | Et |
| 215 | 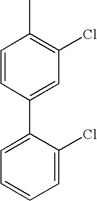 | 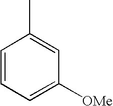 | F | Et |
| 216 | 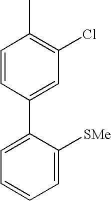 | 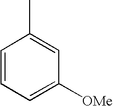 | F | Et |
| 217 | 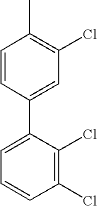 | 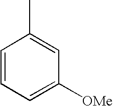 | F | Et |
| 218 | 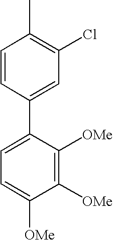 | 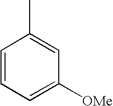 | F | Et |
| 219 | 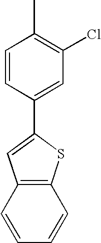 | 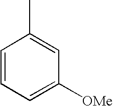 | F | Et |
| 220 | 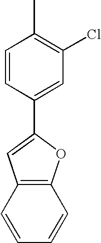 | 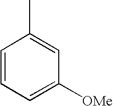 | F | Et |
| 221 | 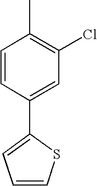 | 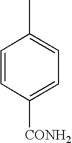 | F | Et |
| 222 | 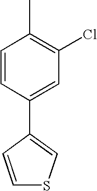 | 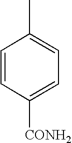 | F | Et |
| 223 | 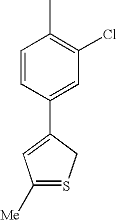 | 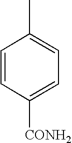 | F | Et |
| 224 | 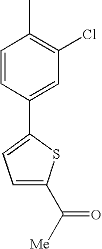 | 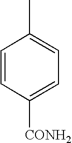 | F | Et |
| 225 | 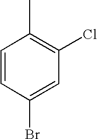 | 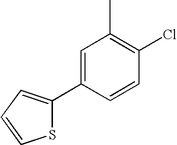 | F | Et |
| 226 | 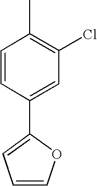 | 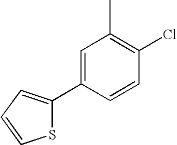 | F | Et |
| 227 | 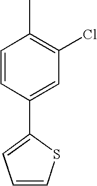 | 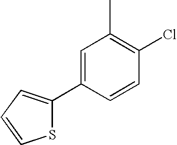 | F | Et |
| 228 | 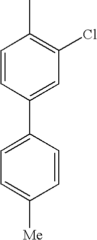 | 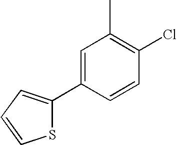 | F | Et |
| 229 | 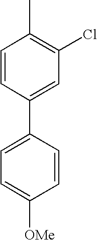 | 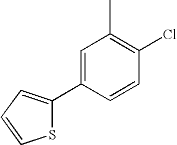 | F | Et |
| 230 | 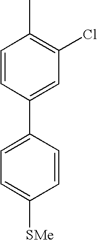 | 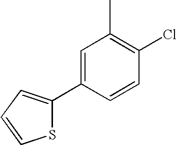 | F | Et |
| 231 | 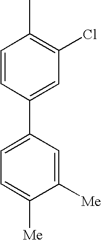 | 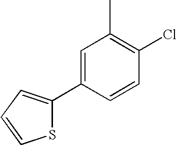 | F | Et |
| 232 | 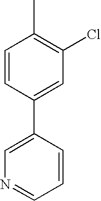 | 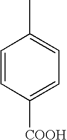 | F | Et |
| 233 | 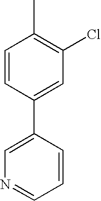 | 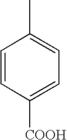 | H | Et |
| 234 | 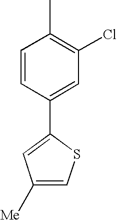 | 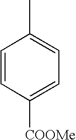 | Br | Et |
| 235 | 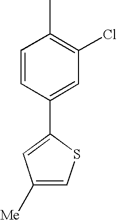 | 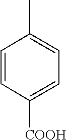 | Br | Et |
| 236 | 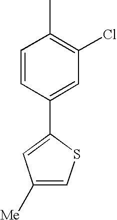 | 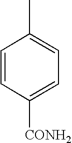 | Br | Et |
| 237 | 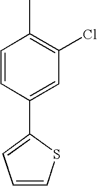 | 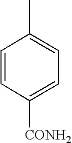 | Br | Et |
| 238 | 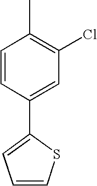 | 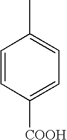 | Br | Et |
| 239 | 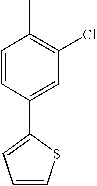 | 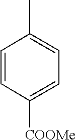 | Br | Et |
| 240 | 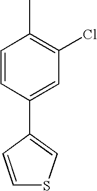 | 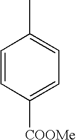 | Br | Et |
| 241 | 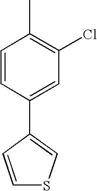 | 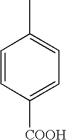 | Br | Et |
| 242 | 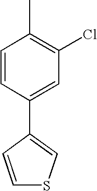 | 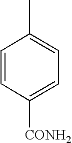 | Br | Et |
| 243 | 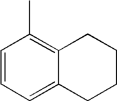 | 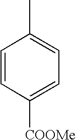 | H | Et |
| 244 | 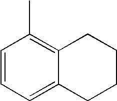 | 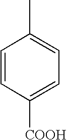 | H | Et |
| 245 | 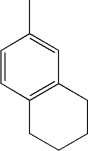 | 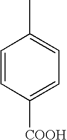 | H | Et |
| 246 | 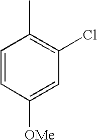 | 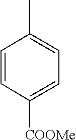 | H | Et |
| 247 | 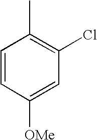 | 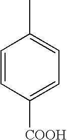 | H | Et |
| 248 | 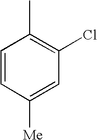 | 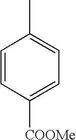 | H | Et |
| 249 | 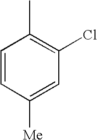 | 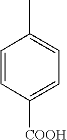 | H | Et |
| 250 | 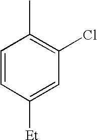 | 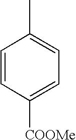 | H | Et |
| 251 | 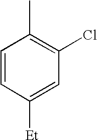 | 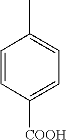 | H | Et |
| 252 | 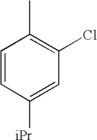 | 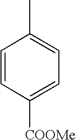 | H | Et |
| 253 | 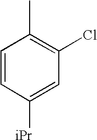 | 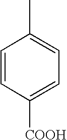 | H | Et |
| 254 | 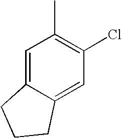 | 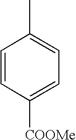 | H | Et |
| 255 | 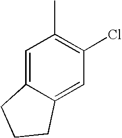 | 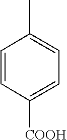 | H | Et |
| 256 | 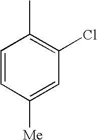 | 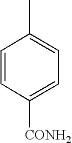 | H | Et |
| 257 | 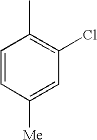 | 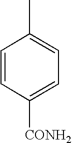 | H | Et |
| 258 | 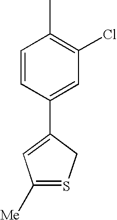 | 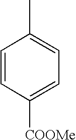 | H | Et |
| 259 | 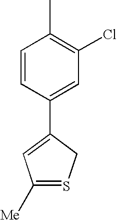 | 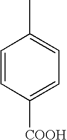 | H | Et |
| 260 | 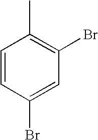 | 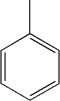 | H | Et |
| 261 | 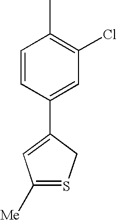 | 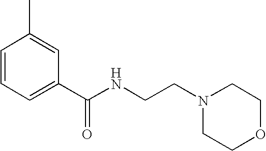 | H | Et |
| 262 | 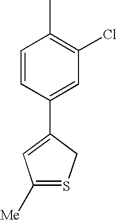 | 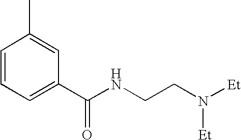 | H | Et |
| 263 | 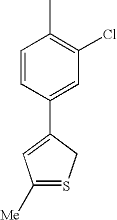 | 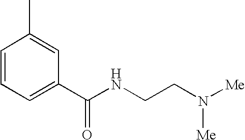 | H | Et |
| 264 | 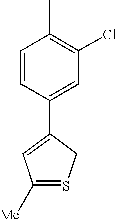 | 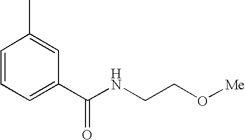 | H | Et |
| 265 | 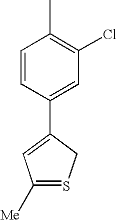 | 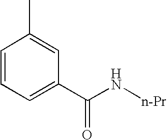 | H | Et |
| 266 | 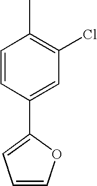 | 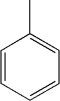 | H | n-Pr |
| 267 | 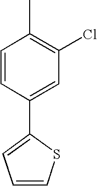 | 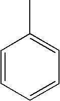 | H | n-Pr |
| 268 | 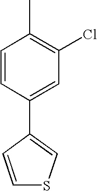 | 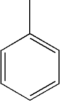 | H | n-Pr |
| 269 | 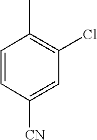 | 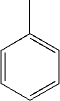 | F | Et |
| 270 | 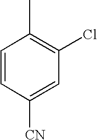 | 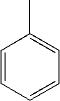 | Br | Et |
| 271 | 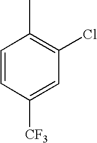 | 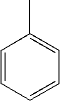 | Br | Et |
| 272 | 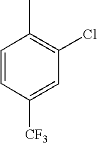 | 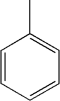 | F | Et |
| 273 | 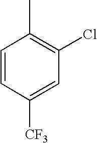 | 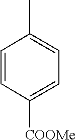 | H | Et |
| 274 | 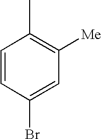 | 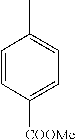 | H | Et |
| 275 | 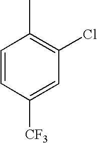 | 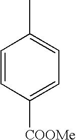 | F | Et |
| 276 | 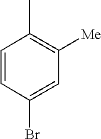 | 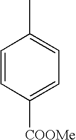 | F | Et |
| 277 | 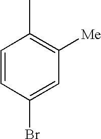 | 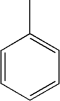 | F | Et |
| 278 | 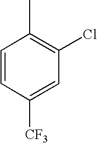 | 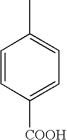 | F | Et |
| 279 | 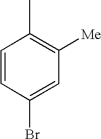 | 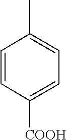 | F | Et |
| 280 | 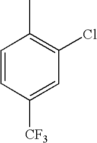 | 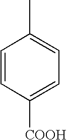 | H | Et |
| 281 | 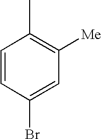 | 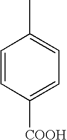 | H | Et |
| 282 | 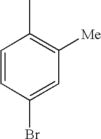 | 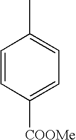 | Br | Et |
| 283 | 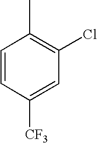 | 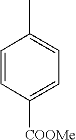 | Br | Et |
| 284 | 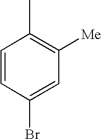 | 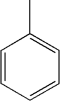 | Br | Et |
| 285 | 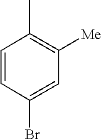 | 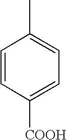 | Br | Et |
| 286 | 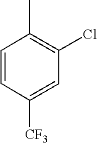 | 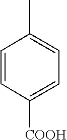 | Br | Et |
| 287 | 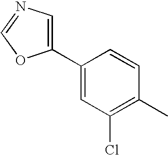 | 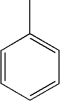 | H | Et |

The compound structures of Table 2 correspond to the IUPAC compound names and characterization data below.

88. 1-{2-[(3-chloro-4′-methoxy-1,1′-biphenyl-4-yl)oxy]ethyl}-5-ethoxy-3-phenyl-1H-pyrazole: HPLC/MS: (M+H)⁺ m/z 449; R_{f}=0.34 (1:4, EtOAc/hexanes); ¹H NMR (CDCl₃): δ 1.45 (t, 3H), 3.85 (s, 3H), 4.2 (q, 2H), 4.45 (s, 4H), 5.8 (s, 1H), 6.95 (m, 3H), 7.3-7.45 (m, 6H), 7.52 (s, 1H), 7.75 (d, 2H).

89. 1-[2-(4-bromo-2-chlorophenoxy)ethyl]-5-ethoxy-3-phenyl-1H-pyrazole: TLC R_{f}=0.7 (3:7, EtOAc/hexanes); ¹H NMR (CDCl₃): δ 1.45 (t, 3H), 4.15 (q, 2H), 4.4 (d, 4H), 5.80 (s, 1H), 6.7 (d, 1H), 7.3-7.35 (m, 2H), 7.40 (t, 2H), 7.47 (s, 1H), 7.75 (d, 2H).

90. 1-{2-[(3-bromo-1,1′-biphenyl-4-yl)oxy]ethyl}-5-ethoxy-3-phenyl-1H-pyrazole: HPLC/MS: M⁺ m/z 463; R_{f}=0.43 (1:4, EtOAc/hexanes); ¹H NMR (CDCl₃): δ 145 (t, 3H), 4.25 (q, 2H), 4.45 (d, 4H), 5.8 (s, 1H), 6.95 (d, 1H), 7.3-7.45 (m, 7H), 7.5 (d, 2H), 7.75 (d, 3H).

91. 1-{2-[(3-chloro-1,1′:4′,1″-terphenyl-4-yl)oxy]ethyl}-5-ethoxy-3-phenyl-1H-pyrazole: HPLC/MS: (M+H)⁺ m/z 496; R_{f}=0.33 (1:4, EtOAc/hexanes); ¹H NMR (CDCl₃): δ 1.42 (t, 3H), 4.2 (q, 2H), 4.45 (d, 4H), 5.8 (s, 1H), 7.0 (d, 1H), 7.28 (m, 3H), 7.4-7.5 (m, 6H), 7.6 (d, 5H), 7.72 (d, 2H).

92. 1-{2-[4-(1-benzothien-2-yl)-2-chlorophenoxy]ethyl}-5-ethoxy-3-phenyl-1H-pyrazole: HPLC/MS: (M+H)⁺ m/z 476; R_{f}=0.3 (1:4, EtOAc/hexanes); ¹H NMR (CDCl₃): δ 1.42 (t, 3H), 4.2 (q, 2H), 4.5 (d, 4H), 5.8 (s, 1H), 6.95 (d, 1H), 7.3-7.5 (m, 8H), 7.6-7.8 (m, 4H).

93. 1-[2-(1,1′-biphenyl-4-yloxy)ethyl]-5-ethoxy-3-phenyl-1H-pyrazole: HPLC/MS: (M)⁺ m/z 385; R_{f}=0.47 (1:4, EtOAc/hexanes); ¹H NMR (CDCl₃): δ 1.4 (t, 3H), 4.2 (q, 2H), 4.4 (s, 4H), 5.85 (s, 1H), 6.95 (d, 2H), 7.3-7.6 (m, 10H), 7.8 (d, 2H).

94. 1-{2-[(3-chloro-2′-fluoro-1,1′-biphenyl-4-yl)oxy]ethyl}-5-ethoxy-3-phenyl-1H-pyrazole: HPLC/MS: (M+H)⁺ m/z 437, R_{f}=0.45 (20% EtOAc/hexanes); ¹H NMR (CDCl₃): δ 1.45 (t, 3H), 4.15 (q, 2H), 4.5 (s, 4H), 5.8 (s, 1H), 6.95 (d, 1H), 7.1-7.4 (m, 8H), 7.6 (s, 1H), 7.75 (d, 2H).

95. 1-{2-[2-chloro-4-(2-naphthyl)phenoxy]ethyl}-5-ethoxy-3-phenyl-1H-pyrazole: HPLC/MS: (M+H)⁺ m/z 469; R_{f}=0.35 (1:4, EtOAc/hexanes); ¹H NMR (CDCl₃): δ 1.48 (t, 3H), 4.17 (q, 2H), 4.5 (s, 4H), 5.83 (s, 1H), 7.0 (d, 1H), 7.3 (m, 1H), 7.35 (t, 2H), 7.5 (d, 3H), 7.65 (s, 1H), 7.75 (m, 3H), 7.90 (m, 3H), 7.97 (s, 1H).

96. 1-{2-[(3,4′-dichloro-3′-fluoro-1, 1′-biphenyl-4-yl)oxy]ethyl}-5-ethoxy-3-phenyl-1H-pyrazole: HPLC/MS: (M+H)⁺ m/z 472; R_{f}=0.29 (1:4, EtOAc/hexanes); ¹H NMR (CDCl₃): δ 1.48 (t, 3H), 4.2 (q, 2H), 4.5 (s, 4H), 5.82 (s, 1H), 6.97 (d, 1H), 7.2 (t, 1H), 7.3-7.4 (m, 5H), 7.5 (d, 2H), 7.77 (d, 2H).

97. 1-{2-[(3-chloro-3′,4′-difluoro-1,1′-biphenyl-4-yl)oxy]ethyl}-5-ethoxy-3-phenyl-1H-pyrazole: HPLC/MS: (M+H)⁺ m/z 455; R_{f}=0.33 (1:4, EtOAc/hexanes); ¹H NMR (CDCl₃): δ 1.48 (t, 3H), 4.17 (q, 2H), 4.7 (s, 4H), 5.85 (s, 1H), 6.86-7.0 (m, 3H), 7.3-7.4 (m, 5H), 7.5 (s, 1H), 7.77 (d, 2H).

98. 1-(2-{[3-chloro-4′-(trifluoromethyl)-1,1′-biphenyl-4-yl]oxy}ethyl)-5-ethoxy-3-phenyl-1H-pyrazole: HPLC/MS: (M+H)⁺ m/z 487; R_{f}=0.33 (1:4, EtOAc/hexanes); ¹H NMR (CDCl₃): δ 1.45 (t, 3H), 4.15 (q, 2H), 4.5 (d, 4H), 5.8 (s, 1H), 7.0 (d, 1H), 7.3 (t, 1H), 7.4 (t, 3H), 7.6 (m, 3H), 7.65 (d, 2H), 7.75 (d, 2H).

99. 1-{2-[(4′-tert-butyl-3-chloro-1,1′-biphenyl-4-yl)oxy]ethyl}-5-ethoxy-3-phenyl-1H-pyrazole: HPLC/MS: (M+H)⁺ m/z 476; R_{f}=0.54 (1:4, EtOAc/hexanes); ¹H NMR (CDCl₃): δ 7.8 (d, 2H), 7.59 (s, 1H), 7.5 (s, 4H), 7.4 (m, 4H), 6.93 (d, 1H), 5.82 (s, 1H), 4.5 (s, 4H), 4.18 (q, 2H), 1.45 (t, 3H), 1.37 (s, 9H).

100. 5-ethoxy-3-phenyl-1-{2-[(3,3′,4′-trichloro-1,1′-biphenyl-4-yl)oxy]ethyl}-1H-pyrazole: HPLC/MS: (M+H)⁺ m/z 488; R_{f}=0.26 (1:4, EtOAc/hexanes); ¹H NMR (CDCl₃): δ 7.78 (d, 2H), 7.3-7.6 (m, 8H), 6.95 (d, 1H), 5.82 (s, 1H), 4.5 (s, 4H), 4.2 (q, 2H), 1.46 (t, 3H).

101. 3-{3-chloro-4-[2-(5-ethoxy-3-phenyl-1H-pyrazol-1-yl)ethoxy]phenyl}pyridine: HPLC/MS: (M+H)⁺ m/z420; R_{f}=0.14 (1:1, EtOAc/hexanes); ¹H NMR (CDCl₃): δ 8.78 (s, 1H), 8.58 (s, 1H), 7.78 (m, 3H), 7.6 (s, 1H), 7.25-7.4 (m, 5H), 7.0 (d, 1H), 5.83 (s, 1H), 4.5 (s, 4H), 4.18 (q, 2H), 1.47 (t, 3H).

102. 1-{2-[2-chloro-4-(3-thienyl)phenoxy]ethyl}-5-ethoxy-3-phenyl-1H-pyrazole: HPLC/MS: (M+H)⁺ m/z 425; R_{f}=0.37 (1:4, EtOAc/hexanes); ¹H NMR (CDCl₃): δ 7.75 (d, 2H), 7.67 (s, 1H), 7.25-7.5 (m, 7H), 7.0 (d, 1H), 5.8 (s, 1H), 4.47 (d, 4H), 4.2 (q, 2H), 1.5 (t, 3H).

103. 1-{2-[2-chloro-4-(5-methyl-3-thienyl)phenoxy]ethyl}-5-ethoxy-3-phenyl-1H-pyrazole: HPLC/MS: (M+H)⁺ m/z 439; R_{f}=0.31 (1:4, EtOAc/hexanes); ¹H NMR (CDCl₃): δ 7.75 (d, 2H), 7.6 (s, 1H), 7.3-7.45 (m, 4H), 7.0 (s, 1H), 6.85 (d, 1H), 6.78 (s, 1H), 5.84 (s, 1H), 4.48 (s, 4H), 4.2 (q, 2H), 2.3 (s, 3H), 1.45 (t, 3H).

104. 1-{2-[2-chloro-4-(2-furyl)phenoxy]ethyl}-5-ethoxy-3-phenyl-1H-pyrazole: HPLC/MS: (M+H)⁺ m/z 409; R_{f}=0.23 (1:4, EtOAc/hexanes); ¹H NMR (CDCl₃): δ 7.65 (t, 2H), 7.6 (d, 1H), 7.2-7.4 (m, 5H), 6.8 (t, 1H), 6.45 (d, 1H), 6.35 (d, 1H), 5.75 (d, 1H), 4.4 (d, 4H), 4.1 (q, 2H), 1.3 (t, 3H).

105. 1-[2-(2,4-difluorophenoxy)ethyl]-5-ethoxy-3-phenyl-1H-pyrazole: HPLC/MS: (M)⁺ m/z 344; TLC (20% Ethyl acetate/hexanes, R_{f}=0.16); ¹H-NMR (CDCl₃): δ 7.7 (d, 2H), 7.25-7.35 (t, 2H), 7.15-7.25 (m, 1H), 6.65-6.85 (m, 2H), 6.60-6.65 (m, 1H), 5.7 (s, 1H), 4.3 (s, 4H), 4.1 (q, 2H), 1.3 (t, 3H).

106. 1-[2-(4-bromo-2-methoxyphenoxy)ethyl]-5-ethoxy-3-phenyl-1H-pyrazole: HPLC/MS: (M)⁺ m/z 417; TLC (20% Ethyl acetate/hexanes, R_{f}=0.15); ¹H-NMR (CDCl₃): δ 7.6 (d, 2H), 7.25-7.35 (m, 2H), 7.15-7.25 (m, 1H), 6.8 (s, 2H), 6.55-6.65 (m, 1H), 5.7 (s, 1H), 4.254.4 (m, 4H), 4.04.1 (q, 2H), 3.75 (s, 3H), 1.35 (t, 3H).

107. 1-[2-(4-chloro-2-fluorophenoxy)ethyl]-5-ethoxy-3-phenyl-1H-pyrazole: HPLC/MS: (M)⁺ m/z 361; TLC (20% Ethyl acetate/hexanes, Rf=0.19); ¹H-NMR (CDCl₃): δ 7.7 (d, 2H), 7.25-7.35 (m, 2H), 7.15-7.25 (m, 1H), 6.95-7.05 (m, 1H), 6.85-6.95 (m, 1H), 6.7-6.85 (m, 1H), 5.7 (s, 1H), 4.3 (s, 4H), 4.1 (q, 2H), 1.35 (t, 3H).

108. 1-[2-(4-bromo-2-fluorophenoxy)ethyl]-5-ethoxy-3-phenyl-1H-pyrazole: HPLC/MS: (M)⁺ m/z 405; TLC (20% Ethyl acetate/hexanes, Rf=0.18); ¹H-NMR (CDCl₃): δ 7.7 (m, 2H), 7.25-7.35 (m, 2H), 7.15-7.25 (m, 1H), 7.15-7.05 (m, 1H), 7.05-6.95 (m, 1H), 6.7-6.85 (m, 1H), 5.7 (s, 1H), 4.3 (s, 4H), 4.1 (q, 2H), 1.35 (t, 3H).

109. 5-ethoxy-1-{2-[(3-iodo-1,1′-biphenyl-4-yl)oxy]ethyl}-3-phenyl-1H-pyrazole: HPLC/MS: (M)⁺ m/z 510; TLC (20% Ethyl acetate/hexanes, R_{f}=0.18); ¹H-NMR (CDCl₃): δ 7.9 (s, 1H), 7.65 (d, 2H), 7.15-7.45 (m, 9H), 6.75 (m, 1H), 5.7 (s, 1H), 4.4 (s, 4H), 4.1 (q, 2H), 1.35 (t, 3H).

110. 1-{2-[2-chloro-4-(trifluoromethyl)phenoxy]ethyl}-5-ethoxy-3-phenyl-1H-pyrazole: LC/MS [M+1]⁺: m/z 411.4, RT 3.57. ¹H-NMR (DMSO, 400 MHz) δ1.40 (m, 3H), 4.30 (m, 2H), 4.40 (s, 2H), 4.71 (s, 2H), 6.30 (s, 1H), 7.39 (m, 1H), 7.45 (m, 3H), 7.70 (s, 1H), 7.90 (m, 2H), 8.18 (s, 1H).

111. 3-chloro-4-[2-(5-ethoxy-3-phenyl-1H-pyrazol-1-yl)ethoxy]benzonitrile: LC/MS [M+1]⁺: m/z 368.39, RT 3.25. ¹H-NMR (DMSO, 400 MHz) δ1.8 (m, 3H), 3.50 (s, 2H), 3.85 (m, 2H), 4.18 (s, 2H), 5.95 (s, 1H), 7.08 (m, 1H), 7.20 (m, 4H), 7.45 (m, 1H), 7.56 (m, 2H).

112. 3-chloro-2-[2-(5-ethoxy-3-phenyl-1H-pyrazol-1-yl)ethoxy]benzoic acid: LC/MS [M+1]⁺: m/z 387.2, RT 3.04

113. 3,5-dichloro-2-[2-(5-ethoxy-3-phenyl-1H-pyrazol-1-yl)ethoxy]benzoic acid: LC/MS [M+1]⁺: m/z 421.1, RT 3.38

114. 3-bromo-4-[2-(5-ethoxy-3-phenyl-1H-pyrazol-1-yl)ethoxy]benzoic acid: LC/MS [M+1]⁺: m/z 431.1, RT 2.92

115. 3-chloro-4-[2-(5-ethoxy-3-phenyl-1H-pyrazol-1-yl)ethoxy]benzoic acid: LC/MS [M+1]⁺: m/z 387, RT 3.04

116. {3-chloro-4-[2-(5-ethoxy-3-phenyl-1H-pyrazol-1-yl)ethoxy]phenyl}acetic acid: LC/MS [M+1]⁺: m/z 401.2, RT 2.96

117. 1-{2-[2-chloro-4-(1H-pyrrol-1-yl)phenoxy]ethyl}-5-ethoxy-3-phenyl-1H-pyrazole: LC/MS [M+1]⁺: m/z 368.39, RT 3.25

118. 5-ethoxy-1-{2-[2-fluoro4-(2-furyl)phenoxy]ethyl}-3-phenyl-1H-pyrazole: HPLC-MS [M+1]⁺: m/z 393, RT 3.62

119. 4-{3-chloro-4-[2-(5-ethoxy-3-phenyl-1H-pyrazol-1-yl)ethoxy]benzyl}morpholine: HPLC-MS [M+1]⁺: m/z 442, RT 2.06

120. 1-{2-[2-chloro-4-( 1-pyrrolidinylmethyl)phenoxy]ethyl}-5-ethoxy-3-phenyl-1H-pyrazole: HPLC-MS [M+1]⁺: m/z 426, RT 2.04

121. N-{3-chloro-4-[2-(5-ethoxy-3-phenyl-1H-pyrazol-1-yl)ethoxy]benzyl}-N,N-diethylamine: HPLC-MS [M+1]⁺: m/z 428, RT 2.04

122. 1-{3-chloro-4-[2-(5-ethoxy-3-phenyl-1H-pyrazol-1-yl)ethoxy]benzyl}piperidine: HPLC-MS [M+1]⁺: m/z 440, RT 2.00

123. 1-[2-(4-bromo-2-methylphenoxy)ethyl]-5-ethoxy-3-phenyl-1H-pyrazole: HPLC-MS [M+1]⁺: m/z 401, RT 3.90

124. 1-[2-(4-bromo-3-methylphenoxy)ethyl]-5-ethoxy-3-phenyl-1H-pyrazole: HPLC-MS [M+1]⁺: m/z 401, RT 3.90

125. 5-ethoxy-1-{2-[2-methyl-4-(2-thienyl)phenoxy]ethyl}-3-phenyl-1H-pyrazole: HPLC-MS [M+1]⁺: m/z 405, RT 4.01

126. 5-ethoxy-1-{2-[3-methyl-4-(2-thienyl)phenoxy]ethyl}-3-phenyl-1H-pyrazole: HPLC-MS [M+1]⁺: m/z 405, RT 4.00

127. 3-chloro-4-[2-(5-ethoxy-4-methyl-3-phenyl-1H-pyrazol-1-yl)ethoxy]benzonitrile: LC/MS [M+1]⁺: m/z 382.5, RT 3.39

128. 1-{2-[2-chloro-4-(trifluoromethyl)phenoxy]ethyl}-5-ethoxy-4-methyl-3-phenyl-1H-pyrazole: LC/MS [M+1]⁺: m/z 425.2, RT 4.04

129. 1-[2-(2-chloro-4-methylphenoxy)ethyl]-5-ethoxy-4-methyl-3-phenyl-1H-pyrazole: LC/MS [M+1]⁺: m/z 371.2, RT 3.93

130. methyl 4-{1-[2-(4-bromo-2-chlorophenoxy)ethyl]-5-ethoxy-1H-pyrazol-3-yl}benzoate: ¹H NMR (CDCl₃): δ 1.41 (t, 3H), 3.92 (s, 3H), 4.10 (q, 2H), 4.40 (m, 4H), 5.82 (s, 1H), 6.75 (d, 1H), 7.25 (m, 1H), 7.42 (s, 1H), 7.78 (d, 2H), 8.01 (d, 2H), LC/MS (M+H)⁺ m/z 479.1

131. 1-[2-(4-bromo-2-chlorophenoxy)ethyl]-5-ethoxy-4-fluoro-3-phenyl-1H-pyrazole: HPLC-MS [M+1]⁺: m/z 439, RT 4.29

132. 1-{2-[2-chloro-4-(2-furyl)phenoxy]ethyl}-5-ethoxy-4-fluoro-3-phenyl-1H-pyrazole: HPLC-MS [M+1]⁺: m/z 427, RT 4.16

133. 1-{2-[2-chloro-4-(2-thienyl)phenoxy]ethyl}-5-ethoxy-4-fluoro-3-phenyl-1H-pyrazole: HPLC-MS [M+1]⁺: m/z 443, RT 4.32

134. 1-{2-[2-chloro-4-(3-thienyl)phenoxy]ethyl}-5-ethoxy-4-fluoro-3-phenyl-1H-pyrazole: HPLC-MS [M+1]⁺: m/z 443, RT 4.23

135. methyl 4-(1-{2-[(3-chloro-4′-methoxy-1,1′-biphenyl-4-yl)oxy]ethyl}-5-ethoxy-1H-p yrazol-3-yl)benzoate): TLC (Rf=0.23, EtOAc-hexanes 1:4); ¹H NMR (CDCl₃): δ 1.42 (t, 3H ), 3.82 (s, 3H), 3.92 (s, 3H), 4.15 (q, 2H), 4.44 (br. s, 4H), 5.85 (s, 1H), 6.85 (m, 3H), 7.28 (d, 1H), 7.39 (d, 2H), 7.54 (d, 1H), 7.80 (d, 2H), 8.03 (d, 2H); LC/MS (M+H)⁺ m/z

136. 4-(1-{2-[2-chloro-4-(3-thienyl)phenoxy]ethyl}-5-ethoxy-1H-pyrazol-3-yl)benzoic acid: TLC (Rf=0.07, EtOAc-hexanes 1:2); ¹H NMR (Acetone): δ 1.42 (t, 3H), 4.15 (q, 2H), 4.43 (t, 2H), 4.53 (t, 2H), 6.20 (s 1H), 7.18 (d, 1H), 7.48 (d, 1H), 7.39 (d, 1H), 7.54 (dd, 1H), 7.70 (m, 2H), 7.92 (d, 2H), 8.03 (d, 2H), 11.15 (br. s, 1H); LC/MS (M+H)⁺ m/z 469.4

137. 4-(1-{2-[2-chloro-4-(2-thienyl)phenoxy]ethyl}-5-ethoxy-1H-pyrazol-3-yl)benzoic acid: TLC (Rf=0.07, EtOAc-hexanes 1:2); ¹H NMR (Acetone): δ 1.42 (t, 3H), 4.15 (q, 2H), 4.43 (t, 2H), 4.53 (t, 2H), 6.20 (s, 1H), 7.08 (dd, 1H), 7.18 (d, 1H), 7.39 (m, 2H), 7.54 (dd, 1H), 7.65 (d, 1H), 7.92 (d, 2H), 8.03 (d, 2H), 11.15 (br. s, 1H); LC/MS (M+H)⁺ m/z 469.4

138. methyl 4-(1-{2-[2-chloro-4-(4-methyl-2-thienyl)phenoxy]ethyl}-5-ethoxy-1H-pyrazol-3-yl)benzoate: TLC (Rf=0.22, EtOAc-hexanes 1:4); ¹H NMR (CDCl₃): δ 1.42 (t, 3H), 2.25 (s, 3H), 3.92 (s, 3H), 4.15 (q, 2H), 4.44 (br. s, 4H), 5.85 (s, 1H), 6.80 (m, 1H), 6.85 (d, 1H), 6.99 (m, 1H), 7.32 (dd, 1H), 7.54 (d, 1H), 7.80 (d, 2H), 8.03 (d, 2H), LC/MS (M+H)⁺ m/z 497.2

139. methyl 4-(1-{2-[4-(5-acetyl-2-thienyl)-2-chlorophenoxy]ethyl}-5-ethoxy-1H-pyrazol-3-yl)benzoate: TLC (Rf=0.15, EtOAc-hexanes 1:2); ¹H NMR (CDCl₃): δ 1.44 (t, 3H), 2.54 (s, 3H), 3.91 (s, 3H), 4.15 (q, 2H), 4.44 (br. s, 4H), 5.85 (s, 1H), 6.88 (d, 1H), 7.18 (d, 1H), 7.40 (d, 1H), 7.62 (m, 2H), 7.81 (d, 2H), 8.02 (d, 2H), LC/MS (M+H)⁺ m/z 525.2

140. methyl 4-(1-{2-[2-chloro-4-(2-naphthyl)phenoxy]ethyl}-5-ethoxy-1H-pyrazol-3-yl)benzoate: TLC (Rf=0.18, EtOAc-hexanes 1:4); ¹H NMR (CDCl₃): δ 1.43 (t, 3H), 3.91 (s, 3H), 4.17 (q, 2H), 4.49 (br. s, 4H), 5.87 (s, 1H), 6.98 (d, 1H), 7.46 (m, 3H), 7.62 (d, 1H), 7.70 (m, 1H), 7.81 (m, 6H), 8.03 (d, 2H), LC/MS (M+H)⁺ m/z 527.4

141. methyl 4-(1-{2-[(3-chloro-2′-fluoro-1,1′-biphenyl-4-yl)oxy]ethyl}-5-ethoxy-1H-pyrazol-3-yl)benzoate: TLC (Rf=0.24, EtOAc-hexanes 1:4); ¹H NMR (CDCl₃): δ

1.41 (t, 3H), 3.91 (s, 3H), 4.15 (q, 2H), 4.47 (br. s, 4H), 5.85 (s, 1H), 6.93 (d, 1H), 7.05 (m, 2H), 7.25 (m, 1H), 7.32 (m, 2H), 7.54 (s, 1H), 7.80 (d, 2H), 8.03 (d, 2H), LC/MS (M+H)⁺ m/z 495.4

142. 4-(1-{2-[2-chloro-4-(4-methyl-2-thienyl)phenoxy]ethyl}-5-ethoxy-1H-pyrazol-3-yl)benzoic acid: ¹H NMR (Acetone): δ 1.41 (t, 3H), 2.11 (s, 3H), 4.20 (q, 2H), 4.41 (t, 2H), 4.53 (t, 2H), 6.19 (s, 1H), 6.99 (s, 1H), 7.17 (s, 1H), 7.19 (d, 1H), 7.50 (d, 1H), 7.60 (s, 1H), 7.90 (d, 2H), 8.03 (d, 2H), 11.15 (br. s), LC/MS (M+H)⁺ m/z 483.2

143. 4-(1-{2-[(3-chloro-4′-methoxy-1,1′-biphenyl-4-yl)oxy]ethyl}-5-ethoxy-1H-pyrazol-3-yl)benzoic acid: ¹H NMR (Acetone): δ 1.41 (t, 3H), 3.82 (s, 3H), 4.20 (q, 2H), 4.42 (t, 2H), 4.53 (t, 2H), 6.19 (s, 1H), 6.99 (d, 2H), 7.09 (d, 1H), 7.45 (m, 4H), 7.90 (d, 2H), 8.03 (d, 2H), 11.15 (br. s, 1H), LC/MS (M+H)⁺ m/z 493.2

144. 4-(1-{2-[4-(5-acetyl-2-thienyl)-2-chlorophenoxy]ethyl}-5-ethoxy-1H-pyrazol-3-yl)benzoic acid: ¹H NMR (Acetone): δ 1.41 (t, 3H), 2.25 (s, 3H), 4.20 (q, 2H), 4.42 (t, 2H), 4.57 (t, 2H), 6.19 (s, 1H), 7.21 (d, 1H), 7.49 (d, 1H), 7.62 (dd, 1H), 7.76 (dd, 2H), 7.90 (d, 2H), 8.03 (d, 2H), 11.15 (br. s, 1H), LC/MS (M+H)⁺ m/z 511.2

145. 1-{2-[(3-chloro-4′-methyl-1,1′-biphenyl-4-yl)oxy]ethyl}-5-ethoxy-4-fluoro-3-phenyl-1H-pyrazole: HPLC-MS m/z (MH+) 451.4, RT 4.42 min

146. 4-(1-{2-[2-chloro-4-(2-naphthyl)phenoxy]ethyl}-5-ethoxy-1H-pyrazol-3-yl)benzoic acid: ¹H NMR (Acetone): δ 1.41 (t, 3H), 4.20 (q, 2H), 4.46 (t, 2H), 4.57 (t, 2H), 6.19 (s, 1H), 7.24 (d, 1H), 7.50 (m, 2H), 7.70 (dd, 1H), 7.78 (m, 3H), 7.90 (m, 6H), 8.08 (s, 1H), 11.15 (br. s, 1H), LC/MS (M+H)⁺ m/z 513.2

147. 4-[1-(2-{[3-chloro-4′-(methylsulfanyl)-1,1′-biphenyl-4-yl]oxy}ethyl)-5-ethoxy-4-fluoro-1H-pyrazol-3-yl]benzoic acid: HPLC-MS m/z (M+) 483.4, RT 4.39 min

148. 1-{2-[(3-chloro-4′-fluoro-1,1′-biphenyl-4-yl)oxy]ethyl}-5-ethoxy-4-fluoro-3-phenyl-1H-pyrazole: HPLC-MS m/z (MH+) 455.4, RT 4.25 min

149. 4-(1-{2-[(3-chloro-2′-fluoro-1,1′-biphenyl-4-yl)oxy]ethyl}-5-ethoxy-1H-pyrazol-3-yl)benzoic acid: ¹H NMR (Acetone): δ 1.41 (t, 3H), 4.20 (q, 2H), 4.44 (t, 2H), 4.57 (t, 2H), 6.19 (s, 1H), 7.18 (m, 3H), 7.35 (m, 1H), 7.45 (m, 2H), 7.58 (s, 1H), 7.92 (d, 2H), 8.03 (d, 2H), 11.15 (br. s, 1H), LC/MS (M+H)⁺ m/z 481.2

150. 1-{2-[(4′-bromo-3-chloro-1,1′-biphenyl-4-yl)oxy]ethyl}-5-ethoxy-4-fluoro-3-phenyl-1H-pyrazole: HPLC-MS m/z (M+) 515.3, RT 4.52 min

151.1-(2-{[3-chloro-4′-(trifluoromethyl)-1,1′-biphenyl-4-yl]oxy}ethyl)-5-ethoxy-4-fluoro-3-phenyl-1H-pyrazole: HPLC-MS m/z (MH+) 505.4, RT 4.45 min

152. 1-{2-[(3-chloro-4′-methoxy-1,1′-biphenyl-4-yl)oxy]ethyl}-5-ethoxy-3-phenyl-1H-pyrazole: HPLC-MS m/z (MH+) 467.4, RT 4.20 min

153. 4-{1-[2-(4-bromo-2-chlorophenoxy)ethyl]-5-ethoxy-1H-pyrazol-3-yl}benzoic acid: ¹H NMR (CD3OD): δ 1.38 (t, 3H), 4.15 (q, 2H), 4.44 (m, 2H), 4.57 (t, 2H), 6.10 (s, 1H), 6.99 (d, 1H), 7.31 (dd, 1H), 7.48 (d, 1H), 7.80 (d, 2H), 8.03 (d, 2H) carboxylic acid H not visible, LC/MS (M+H)⁺ m/z 465.1

154. methyl 4-(1-{2-[(3-chloro-2′,4′-difluoro-1,1′-biphenyl-4-yl)oxy]ethyl}-5-ethoxy-1H-pyrazol-3-yl)benzoate: ¹H NMR (CDCl3): δ 1.38 (t, 3H), 3.92 (s, 3H), 4.10 (q, 2H), 4.44 (br. s, 4H), 5.82 (s, 1H), 6.91 (m, 3H), 7.12 (m, 2H), 7.44 (s, 1H), 7.80 (d, 2H), 8.03 (d, 2H), LC/MS (M+H)⁺ m/z 513.4

155. methyl 4-(1-{2-[(3,3′-dichloro-4′-fluoro-1,1′-biphenyl-4-yl)oxy]ethyl}-5-ethoxy-1H-pyrazol-3-yl)benzoate: ¹H NMR (CDCl3): δ 1.38 (t, 3H), 3.92 (s, 3H), 4.10 (q, 2H), 4.44 (br. s, 4H), 5.82 (s, 1H), 6.91 (d, 1H), 7.10 (t, 1H), 7.22 (m, 2H), 7.45 (m, 2H), 7.90 (d, 2H), 8.03 (d, 2H), LC/MS (M+H)⁺ m/z 513.4

156. methyl 4-(1-{2-[(3-chloro-2′-fluoro-1,1′:4′,1″-terphenyl-4-yl)oxy]ethyl}-5-ethoxy-1H-pyrazol-3-yl)benzoate: ¹H NMR (CDCl3): δ 1.40 (t, 3H), 3.92 (s, 3H), 4.10 (q, 2H), 4.44 (br. s, 4H), 5.82 (s, 1H), 6.95 (d, 1H), 7.22 (m, 7H), 7.58 (m, 3H), 7.80 (d, 2H), 8.03 (d, 2H), LC/MS (M+H)⁺ m/z 571.5

157. 4-(1-{2-[(3-chloro-2′,4′-difluoro-1,1′-biphenyl-4-yl)oxy]ethyl}-5-ethoxy-1H-pyrazol-3-yl)benzoic acid: ¹H NMR (Acetone): δ 1.38 (t, 3H), 4.20 (q, 2H), 4.42 (t, 2H), 4.57 (t, 2H), 6.19 (s, 1H), 7.05 (m, 2H), 7.21 (d, 1H), 7.40 (dd, 1H), 7.50 (m, 2H), 7.90 (d, 2H), 8.03 (d, 2H), 11.15 (br. s, 1H), LC/MS (M+H)⁺ m/z 499.2

158. 4-(1-{2-[(3,3′-dichloro-4′-fluoro-1,1′-biphenyl-4-yl)oxy]ethyl}-5-ethoxy-1H-pyrazol-3-yl)benzoic acid: ¹H NMR (Acetone): δ 1.38 (t, 3H), 4.20 (q, 2H), 4.42 (t, 2H), 4.57 (t, 2H), 6.19 (s, 1H), 7.21 (d, 1H), 7.35 (d, 1H), 7.55 (m, 2H), 7.65 (s, 1H), 7.75 (d, 1H), 7.90 (d, 2H), 8.03 (d, 2H), 11.15 (br. s, 1H), LC/MS (M+H)⁺ m/z 515.2

159. 4-(1-{2-[(3-chloro-2′-fluoro-1,1′:4′,1″-terphenyl-4-yl)oxy]ethyl}-5-ethoxy-1H-pyrazol-3-yl)benzoic acid: ¹H NMR (Acetone): δ 1.38 (t, 3H), 4.20 (q, 2H), 4.42 (t, 2H), 4.57 (t, 2H), 6.19 (s, 1H), 7.25 (d, 1H), 7.38 (d, 1H), 7.42 (m, 8H), 7.75 (s, 1H), 7.90 (d, 2H), 8.03 (d, 2H), 11.15 (br. s, 1H), LC/MS (M+H)⁺ m/z 557.2

160. 1-[2-(5-bromo-2-chlorophenoxy)ethyl]-5-ethoxy-3-phenyl-1H-pyrazole: HPLC-MS [M+1]⁺: m/z 421, RT 3.95

161. 1-{2-[2-chloro-5-(2-furyl)phenoxy]ethyl}-5-ethoxy-3-phenyl-1H-pyrazole: HPLC-MS [M+1]⁺: m/z 409, RT 3.76

162. 1-{2-[2-chloro-5-(3-thienyl)phenoxy]ethyl}-5-ethoxy-3-phenyl-1H-pyrazole: HPLC-MS [M+1]⁺: m/z 425, RT 3.93

163. 1-{2-[2-chloro-5-(1H-pyrrol-2-yl)phenoxy]ethyl}-5-ethoxy-3-phenyl-1H-pyrazole: HPLC-MS [M+1]⁺: m/z 408, RT 3.83

164. methyl 4-(1-{2-[2-chloro-4-(2-thienyl)phenoxy]ethyl}-5-ethoxy-4-fluoro-1H-pyrazol-3-yl)benzoate: TLC (Rf=0.43, EtOAc-hexanes 1:4); ¹H NMR (CDCl3): δ 1.41 (t, 3H), 3.92 (s, 3H), 4.40 (m, 6H), 6.90 (d, 1H), 7.01 (dd, 1H), 7.18 (d, 1H), 7.21 (d, 1H), 7.38 (dd, 1H), 7.60 (s, 1H), 7.90 (d, 2H), 8.03 (d, 2H), LC/MS (M+H)⁺ m/z 501.1

165. methyl 4-(1-{2-[2-chloro-4-(3-thienyl)phenoxy]ethyl}-5-ethoxy-4-fluoro-1H-pyrazol-3-yl)benzoate: TLC (Rf=0.39, EtOAc-hexanes 1:4); ¹H NMR (CDCl3): δ 1.41 (t, 3H), 3.92 (s, 3H), 4.40 (m, 6H), 6.90 (d, 1H), 7.25 (m, 1H), 7.30 (m, 3H), 7.60 (s, 1H), 7.90 (d, 2H), 8.03 (d, 2H), LC/MS (M+H)⁺ m/z 501.1

166. methyl 4-(1-{2-[2-chloro-4-(4-methyl-2-thienyl)phenoxy]ethyl}-5-ethoxy-4-fluoro-1H-pyrazol-3-yl)benzoate: TLC (Rf=0.45, EtOAc-hexanes 1:4); ¹H NMR (CDCl3): δ 1.41 (t, 3H), 2.23 (s, 3H), 3.92 (s, 3H), 4.40 (m, 6H), 6.81 (s, 1H), 6.90 (d, 1H), 7.00 (s, 1H), 7.30 (dd, 1H), 7.55 (s, 1H), 7.90 (d, 2H), 8.03 (d, 2H), LC/MS (M+H)⁺ m/z 511.1

167. methyl 4-(1-{2-[4-(5-acetyl-2-thienyl)-2-chlorophenoxy]ethyl}-5-ethoxy-4-fluoro-1H-pyrazol-3-yl)benzoate: TLC (Rf=0.11, EtOAc-hexanes 1:4); ¹H NMR (CDCl3): δ 1.41 (t, 3H), 2.53 (s, 3H), 3.92 (s, 3H), 4.40 (m, 6H), 6.90 (d, 1H), 7.18 (d, 1H), 7.41 (dd, 1H), 7.60 (m, 2H), 7.90 (d, 2H), 8.03 (d, 2H), LC/MS (M+H)⁺ m/z 543.0

168. 4-(1-{2-[2-chloro-4-(4-methyl-2-thienyl)phenoxy]ethyl}-5-ethoxy-4-fluoro-1H-pyrazol-3-yl)benzoic acid: ¹H NMR (Acetone): δ 1.41 (t, 3H), 2.53 (s, 3H), 4.40 (t, 4H), 4.60 (t, 2H), 7.25 (d, 1H), 7.50 (d, 1H), 7.63 (dd, 1H), 7.75 (d, 1H), 7.80 (d, 1H), 7.90 (d, 2H), 8.05 (d, 2H), 11.15 (br. s, 1H), LC/MS (M+H)⁺ m/z 501.1

169. 4-( 1-{2-[4-(5-acetyl-2-thienyl)-2-chlorophenoxy]ethyl}-5-ethoxy-4-fluoro-1H-pyrazol-3-yl)benzoic acid: ¹H NMR (Acetone): δ 1.41 (t, 3H), 2.23 (s, 3H), 4.40 (m, 4H), 4.58 (t, 2H), 6.99 (s, 1H), 7.19 (s, 1H), 7.50 (dd, 1H), 7.60 (d, 1H), 7.90 (d, 2H), 8.05 (d, 2H), 11.15 (br. s, 1H), LC/MS (M+H)⁺ m/z 529.1

170. 4-(1-{2-[2-chloro-4-(2-thienyl)phenoxy]ethyl}-5-ethoxy-4-fluoro-1H-pyrazol-3-yl)benzoic acid: ¹H NMR (Acetone): δ 1.41 (t, 3H), 4.40 (m, 4H), 4.58 (t, 2H), 7.08 (dd, 1H), 7.19 (d, 1H), 7.38 (m, 2H), 7.55 (dd, 1H), 7.65 (s, 1H), 7.90 (d, 2H), 8.05 (d, 2H), carboxylic acid H not observed, LC/MS (M+H)⁺ m/z 487.1

171. 4-(1-{2-[2-chloro-4-(3-thienyl)phenoxy]ethyl}-5-ethoxy-4-fluoro-1H-pyrazol-3-yl)benzoic acid: ¹H NMR (Acetone): δ 1.41 (t, 3H), 4.40 (m, 4H), 4.58 (t, 2H), 7.08 (dd, 1H), 7.45 (d, 1H), 7.55 (m, 1H), 7.60 (dd, 1H), 7.70 (m, 2H), 7.90 (d, 2H), 8.05 (d, 2H), carboxylic acid H not observed, LC/MS (M+H)⁺ m/z 487.1

172. 1-[2-(4-bromo-2-chlorophenoxy)ethyl]-5-ethoxy-4-fluoro-3-(3-methylphenyl)-1H-pyrazole: HPLC-MS m/z (MH+) 451.4, ret. time 4.42 min

173. 1-{2-[2-chloro-4-(2-furyl)phenoxy]ethyl}-5-ethoxy-4-fluoro-3-(3-methylphenyl)-1H-pyrazole: HPLC-MS m/z (MH+) 441.2, ret. time 4.45 min

174. 1-{2-[2-chloro-4-(3-thienyl)phenoxy]ethyl}-5-ethoxy-4-fluoro-3-(3-methylphenyl)-1H-pyrazole: HPLC-MS m/z (MH+) 457.1, ret. time 4.52 min

175. 1-{2-[(3-chloro-4′-methyl-1,1′-biphenyl-4-yl)oxy]ethyl}-5-ethoxy4-fluoro-3-(3-methylphenyl)-1H-pyrazole: HPLC-MS m/z (MH+) 465.2, ret. time 4.82 min

176. 1-(2-{[3-chloro-4′-(methylsulfanyl)-1,1′-biphenyl-4-yl]oxy}ethyl)-5-ethoxy-4-fluoro-3-(3-methylphenyl)-1H-pyrazole: HPLC-MS m/z (M+) 497.2, ret. time 4.78 min

177. 1-{2-[(3-chloro-4′-methoxy-1,1′-biphenyl-4-yl)oxy]ethyl}-5-ethoxy-4-fluoro-3-(3-methylphenyl)-1H-pyrazole: HPLC-MS m/z (MH+) 481.2, ret. time 4.58 min

178. 3′-chloro-4′-{2-[5-ethoxy-4-fluoro-3-(3-methylphenyl)-1H-pyrazol-1-yl]ethoxy}-1,1′-biphenyl-3-amine: HPLC-MS m/z (MH+) 466.2, ret. time 3.35 min

179. 1-{2-[4-(1,3-benzodioxol-5-yl)-2-chlorophenoxy]ethyl}-5-ethoxy-4-fluoro-3-(3-methylphenyl)-1H-pyrazole: HPLC-MS m/z (MH+) 495.2, ret. time 4.51 min

180. 1-{2-[(3-chloro-2′-methoxy-1,1′-biphenyl-4-yl)oxy]ethyl}-5-ethoxy-4-fluoro-3-(3-methylphenyl)-1H-pyrazole: HPLC-MS m/z (MH+) 481.2, ret. time 4.58 min

181. 1-{2-[(3-chloro-3′,4′-dimethyl-1,1′-biphenyl-4-yl)oxy]ethyl}-5-ethoxy-4-fluoro-3-(3-methylphenyl)-1H-pyrazole: HPLC-MS m/z (MH+) 479.2, ret. time 4.94 min

182. 1-{2-[(2′,3-dichloro-1,1′-biphenyl-4-yl)oxy]ethyl}-5-ethoxy-4-fluoro-3-(3-methylphenyl)-1H-pyrazole: HPLC-MS m/z (M+) 485.1, ret. time 4.75 min

183. 1-(2-{[3-chloro-2′-(methylsulfanyl)-1,1′-biphenyl-4-yl]oxy}ethyl)-5-ethoxy-4-fluoro-3-(3-methylphenyl)-1H-pyrazole: HPLC-MS m/z (M+) 497.2, ret. time 4.68 min

184. 5-ethoxy-4-fluoro-3-(3-methylphenyl)-1-{2-[(2′,3,3′-trichloro-1,1′-biphenyl-4-yl)oxy]ethyl}-1H-pyrazole: HPLC-MS m/z (M+) 519.1, 521.1, ret. time 4.93 min

185. 1-{2-[(3-chloro-2′,3′,4′-trimethoxy-1,1′-biphenyl-4-yl)oxy]ethyl}-5-ethoxy-4-fluoro-3-(3-methylphenyl)-1H-pyrazole: HPLC-MS m/z (M+) 541.2, ret. time 4.33 min

186. 1-{2-[4-(1-benzothien-2-yl)-2-chlorophenoxy]ethyl}-5-ethoxy-4-fluoro-3-(3-methylphenyl)-1H-pyrazole: HPLC-MS m/z (M+) 507.1, ret. time 4.99 min

187.1-{2-[4-(1-benzofuran-2-yl)-2-chlorophenoxy]ethyl}-5-ethoxy-4-fluoro-3-(3-methylphenyl)-1H-pyrazole: HPLC-MS m/z (MH+) 491.2, ret. time 4.89 min

188. 1-[2-(4-bromo-2-chlorophenoxy)ethyl]-5-ethoxy-4-fluoro-3-(3-methoxyphenyl)-1H-pyrazole: HPLC-MS, m/z469.1 (M+1), RT 4.28 min

189. 1-{2-[2-chloro-5-(2-thienyl)phenoxy]ethyl}-5-ethoxy-3-phenyl-1H-pyrazole: HPLC-MS [M+1]⁺: m/z 425, RT 4.18

190. 1-{2-[2-chloro-4-(1H-pyrrol-2-yl)phenoxy]ethyl}-5-ethoxy-3-phenyl-1H-pyrazole: HPLC-MS [M+1]⁺: m/z 408, RT 3.75

191. 5-bromo-2-[2-(5-ethoxy-3-phenyl-1H-pyrazol-1-yl)ethoxy]benzonitrile: HPLC-MS [M+1]⁺: m/z 412, RT 3.69

192. 4-[1-(2-{[3-chloro-4′-(methylsulfanyl)-1,1′-biphenyl-4-yl]oxy}ethyl)-5-ethoxy-1H-pyrazol-3-yl]benzoic acid: ¹H NMR (Acetone): δ 1.41 (t, 3H), 2.51 (s, 3H), 4.20 (q, 2H), 4.42 (t, 2H), 4.58 (t, 2H), 6.19 (s, 1H), 7.19 (d, 1H), 7.32 (d, 2H), 7.55 (m, 3H), 7.60 (s, 1H), 7.90 (d, 2H), 8.03 (d, 2H), 11.15 (br.s, 1H), LC/MS (M+H)⁺ m/z 509.2

193. methyl 4-[1-(2-{[3-chloro-4′-(methylsulfanyl)-1,1′-biphenyl-4-yl]oxy}ethyl)-5-ethoxy-1H-pyrazol-3-yl]benzoate: ¹H NMR (CDCl3): δ 1.41 (t, 3H), 2.51 (s, 3H), 3.92 (s, 3H), 4.15 (q, 2H), 4.45 (br.s, 4H), 5.85 (s, 1H), 6.90 (d, 1H), 7.25 (m, 3H), 7.38 (d, 2H), 7.55 (s, 1H), 7.90 (d, 2H), 8.03 (d, 2H), LC/MS (M+H)^{+ m/z} 523.2

194. 1-[2-(2-chlorophenoxy)ethyl]-5-ethoxy-4-fluoro-3-(3-methoxyphenyl)-1H-pyrazole: HPLC-MS m/z (MH+) 391.2, RT 4.02 min

195. 5-ethoxy-4-fluoro-3-(3-methoxyphenyl)-1-[2-(2,3,4-trichlorophenoxy)ethyl]-1H-pyrazole: HPLC-MS m/z (M+) 459.1, 461.0 RT 4.44 min

196. N-(3-chloro-4-{2-[5-ethoxy-4-fluoro-3-(3-methoxyphenyl)-1H-pyrazol-1-yl]ethoxy}phenyl)acetamide: HPLC-MS m/z (MH+) 448.1, RT 3.43 min

197. 1-[2-(4-tert-butyl-2-chlorophenoxy)ethyl]-5-ethoxy-4-fluoro-3-(3-methoxyphenyl)-1H-pyrazole: HPLC-MS m/z (MH+) 447.2, RT 4.62 min

198. 1-[2-(2,3-dichlorophenoxy)ethyl]-5-ethoxy-4-fluoro-3-(3-methoxyphenyl)-1H-pyrazole: HPLC-MS m/z (M+) 425.1, RT 4.22 min

199. 1-{2-[2-chloro-4-(trifluoromethyl)phenoxy]ethyl}-5-ethoxy-4-fluoro-3-(3-methoxyphenyl)-1H-pyrazole: HPLC-MS m/z (MH+) 459.2, RT 4.30 min

200. 3-chloro-4-{2-[5-ethoxy-4-fluoro-3-(3-methoxyphenyl)-1H-pyrazol-1-yl]ethoxy}benzonitrile: HPLC-MS m/z (MH+) 416.1, RT 3.85 min

201. 1-[2-(2-bromo-4-chlorophenoxy)ethyl]-5-ethoxy-4-fluoro-3-(3-methoxyphenyl)-1H-pyrazole: HPLC-MS m/z (M+) 469.1, 471.0, RT 4.33 min

202. 1-{2-[2-chloro-3-(trifluoromethyl)phenoxy]ethyl}-5-ethoxy-4-fluoro-3-(3-methoxyphenyl)-1H-pyrazole: HPLC-MS m/z (MH+) 459.1, RT 4.23 min

203. 1-[2-(2,3-dichloro-4-methoxyphenoxy)ethyl]-5-ethoxy-4-fluoro-3-(3-methoxyphenyl)-1H-pyrazole: HPLC-MS m/z (M+) 455.1, RT 4.13 min

204. 2-[2-(5-ethoxy-3-phenyl-1H-pyrazol-1-yl)ethoxy]-5-(3-thienyl)benzonitrile: HPLC-MS [M+1]⁺: m/z 416, RT 3.91

205. 2-[2-(5-ethoxy-3-phenyl-1H-pyrazol-1-yl)ethoxy]-5-(2-furyl)benzonitrile: HPLC-MS [M+1]⁺: m/z 400, RT 3.42

206. 2-[2-(5-ethoxy-3-phenyl-1H-pyrazol-1-yl)ethoxy]-5-(3-pyridinyl)benzonitrile: HPLC-MS [M+1]⁺: m/z 411, RT 2.76

207. 1-{2-[2-chloro-4-(2-furyl)phenoxy]ethyl}-5-ethoxy-4-fluoro-3-(3-methoxyphenyl)-1H-pyrazole: HPLC-MS m/z (MH+) 457.4, RT 4.00 min

208. 1-{2-[2-chloro-4-(3-thienyl)phenoxy]ethyl}-5-ethoxy-4-fluoro-3-(3-methoxyphenyl)-1H-pyrazole: HPLC-MS m/z (MH+) 473.4, RT 4.06 min

209. 1-{2-[(3-chloro-4′-methyl-1,1′-biphenyl-4-yl)oxy]ethyl}-5-ethoxy-4-fluoro-3-(3-methoxyphenyl)-1H-pyrazole: HPLC-MS m/z (MH+) 481.5, RT 4.34 min

210. 1-(2-{[3-chloro-4′-(methylsulfanyl)-1,1′-biphenyl-4-yl]oxy}ethyl)-5-ethoxy-4-fluoro-3-(3-methoxyphenyl)-1H-pyrazole: HPLC-MS m/z (M+) 513.4, RT 4.30 min

211. 1-{2-[(3-chloro-4′-methoxy-1,1′-biphenyl-4-yl)oxy]ethyl}-5-ethoxy-4-fluoro-3-(3-methoxyphenyl)-1H-pyrazole: HPLC-MS m/z (MH+) 497.5, RT 4.11 min

212. 1-{2-[4-(1,3-benzodioxol-5-yl)-2-chlorophenoxy]ethyl}-5-ethoxy-4-fluoro-3-(3-methoxyphenyl)-1H-pyrazole: HPLC-MS m/z (MH+) 511.5, RT 4.06 min

213. 1-{2-[(3-chloro-2′-methoxy-1,1′-biphenyl-4-yl)oxy]ethyl}-5-ethoxy-4-fluoro-3-(3-methoxyphenyl)-1H-pyrazole: HPLC-MS m/z (MH+) 497.6, RT 4.11 min

214. 1-{2-[(3-chloro-3′,4′-dimethyl-1,1′-biphenyl-4-yl)oxy]ethyl}-5-ethoxy-4-fluoro-3-(3-methoxyphenyl)-1H-pyrazole: HPLC-MS m/z (MH+) 495.6, RT 4.46 min

215. 1-{2-[(2′,3-dichloro-1,1′-biphenyl-4-yl)oxy]ethyl}-5-ethoxy-4-fluoro-3-(3-methoxyphenyl)-1H-pyrazole: HPLC-MS m/z (M+) 501.5, RT 4.28 min

216. 1-(2-{[3-chloro-2′-(methylsulfanyl)-1,1′-biphenyl-4-yl]oxy}ethyl)-5-ethoxy-4-fluoro-3-(3-methoxyphenyl)-1H-pyrazole: HPLC-MS m/z (M+) 513.5, RT 4.22 min

217. 5-ethoxy-4-fluoro-3-(3-methoxyphenyl)-1-{2-[(2′,3,3′-trichloro-1,1′-biphenyl-4-yl)oxy]ethyl}-1H-pyrazole: HPLC-MS m/z (M+) 535.3, 537.3 RT 4.45 min

218. 1-{2-[(3-chloro-2′,3′,4′-trimethoxy-1,1′-biphenyl-4-yl)oxy]ethyl}-5-ethoxy-4-fluoro-3-(3-methoxyphenyl)-1H-pyrazole: HPLC-MS m/z (M+) 557.7, RT 3.90 min

219. 1-{2-[4-(1-benzothien-2-yl)-2-chlorophenoxy]ethyl}-5-ethoxy-4-fluoro-3-(3-methoxyphenyl)-1H-pyrazole: HPLC-MS m/z (M+) 523.3, RT 4.51 min

220. 1-{2-[4-(1-benzofuran-2-yl)-2-chlorophenoxy]ethyl}-5-ethoxy-4-fluoro-3-(3-methoxyphenyl)-1H-pyrazole: HPLC-MS m/z (MH+) 507.3, RT 4.41 min

221. 4-(1-{2-[2-chloro-4-(2-thienyl)phenoxy]ethyl}-5-ethoxy-4-fluoro-1H-pyrazol-3-yl)benzamide: ¹H NMR (CDCl3): δ 1.41 (t, 3H), 4.40 (m, 6H), 6.85 (d, 1H), 7.05 (m, 1H), 7.20 (s, 1H), 7.25 (m, 2H), 7.38 (d, 2H), 7.60 (s, 1H), 7.80 (m, 4H), LC/MS (M+H)⁺ m/z 486.1

222. 4-(1-{2-[2-chloro-4-(3-thienyl)phenoxy]ethyl}-5-ethoxy-4-fluoro-1H-pyrazol-3-yl)benzamide: ¹H NMR (CDCl3): δ 1.38 (t, 3H), 4.25 (m, 6H), 6.85 (d, 1H), 7.05 (m, 1H), 7.55 (s, 1H), 7.40 (m, 5H), 7.80 (m, 4H), LC/MS (M+H)⁺ m/z 486.1

223. 4-(1-{2-[2-chloro-4-(4-methyl-2-thienyl)phenoxy]ethyl}-5-ethoxy-4-fluoro-1H-pyrazol-3-yl)benzamide: ¹H NMR (CDCl3): δ 1.41 (t, 3H), 2.23 (m, 3H), 4.35 (m, 6H), 6.80 (s, 1H), 6.90 (d, 1H), 7.00 (s, 1H), 7.30 (m, 1H), 7.55 (s, 1H), 7.80 (4H), LC/MS (M+H)⁺ m/z 500.1

224. 4-(1-{2-[4-(5-acetyl-2-thienyl)-2-chlorophenoxy]ethyl}-5-ethoxy-4-fluoro-1H-pyrazol-3-yl)benzamide: ¹H NMR (CDCl3): δ 1.41 (t, 3H), 2.55 (m, 3H), 4.40 (m, 6H), 6.90 (d, 1H), 7.18 (s, 1H), 7.42 (s, 1H), 7.60 (m, 2H), 7.80 (m, 4H), LC/MS (M+H)⁺ m/z 527.9

225. 1-[2-(4-bromo-2-chlorophenoxy)ethyl]-3-(3,5-dimethylphenyl)-5-ethoxy-4-fluoro-1H-pyrazole: HPLC-MS m/z (MH+) 467.1, 469.1, RT 4.60 min

226. 1-{2-[2-chloro-4-(2-furyl)phenoxy]ethyl}-3-(3,5-dimethylphenyl)-5-ethoxy-4-fluoro-1H-pyrazole: HPLC-MS m/z (MH+) 455.2, RT 4.57 min

227. 1-{2-[2-chloro-4-(3-thienyl)phenoxy]ethyl}-3-(3,5-dimethylphenyl)-5-ethoxy-4-fluoro-1H-pyrazole: HPLC-MS m/z (MH+) 471.2, RT 4.62 min

228. 1-{2-[(3-chloro-4′-methyl-1,1′-biphenyl-4-yl)oxy]ethyl}-3-(3,5-dimethylphenyl)-5-ethoxy-4-fluoro-1H-pyrazole: HPLC-MS m/z (MH+) 479.2, RT 4.89 min

229. 1-{2-[(3-chloro-4′-methoxy-1,1′-biphenyl-4-yl)oxy]ethyl}-3-(3,5-dimethylphenyl)-5-ethoxy-4-fluoro-1H-pyrazole: HPLC-MS m/z (MH+) 495.2, RT 4.68 min

230. 1-(2-{[3-chloro-4-(methylsulfanyl)-1,1′-biphenyl-4-yl]oxy}ethyl)-3-(3,5-dimethylphenyl)-5-ethoxy-4-fluoro-1H-pyrazole: HPLC-MS m/z (M+) 511.2, RT 4.87 min

231. 1-{2-[(3-chloro-3′,4′-dimethyl-1,1′-biphenyl-4-yl)oxy]ethyl}-3-(3,5-dimethylphenyl)-5-ethoxy-4-fluoro-1H-pyrazole: HPLC-MS m/z (M+) 493.2, RT 5.02 min

232. 4-(1-{2-[2-chloro-4-(3-pyridinyl)phenoxy]ethyl}-5-ethoxy-4-fluoro-1H-pyrazol-3-yl)benzoic acid: ¹H NMR (CD3OD): δ 1.38 (t, 3H), 4.40 (m, 4H), 4.55 (m, 2H), 7.25 (d, 1H), 7.65 (d, 1H), 7.80 (m, 3H), 8.00 (d, 2H), 8.10 (dd, 1H), 8.75 (d, 1H), 8.80 (d, 1H), 9.00 (1H), (LC/MS (M+H)⁺ m/z 482.2

233. 4-(1-{2-[2-chloro-4-(3-pyridinyl)phenoxy]ethyl}-5-ethoxy-1H-pyrazol-3-yl)benzoic acid: ¹H NMR (CD3OD): δ 1.38 (t, 3H), 4.20 (q, 2H), 4.45 (m, 4H), 6.25 (s, 1H), 7.25 (d, 1H), 7.65 (d, 1H), 7.80 (m, 3H), 8.00 (d, 2H), 8.10 (dd, 1H), 8.75 (d, 1H), 8.80 (d, 1H), 9.00 (1H), carboxylic acid H not seen (LC/MS (M+H)⁺ m/z 464.2

234. methyl 4-(4-bromo-1-{2-[2-chloro-4-(4-methyl-2-thienyl)phenoxy]ethyl}-5-ethoxy-1H-pyrazol-3-yl)benzoate: ¹H NMR (CDCl3): δ 1.41 (t, 3H), 2.25 (s, 3H), 3.92 (s, 3H), 4.45 (m, 6H), 6.80 (s, 1H), 6.90 (d, 1H), 7.00 (s, 1H), 7.35 (d, 1H), 7.55 (s, 1H), 7.90 (d, 2H), 8.10 (d, 2H), (LC/MS (M+H)⁺ m/z 575.3

235. 4-(4-bromo-1-{2-[2-chloro-4-(4-methyl-2-thienyl)phenoxy]ethyl}-5-ethoxy-1H-pyrazol-3-yl)benzoic acid: ¹H NMR (Acetone): δ 1.41 (t, 3H), 2.23 (s, 3H), 4.40 (m, 4H), 4.60 (m, 2H), 6.99 (d, 1H), 7.20 (m, 1H), 7.35 (d, 1H), 7.50 (d, 1H), 7.60 (d, 1H), 7.95 (d, 2H), 8.10 (d, 2H),carboxylic acid H not seen, (LC/MS (M+H)⁺ m/z 561.1

236. 4-(4-bromo-1-{2-[2-chloro-4-(4-methyl-2-thienyl)phenoxy]ethyl}-5-ethoxy-1H-pyrazol-3-yl)benzamide: ¹H NMR (CDCl3): δ 1.38 (t, 3H), 2.20 (s, 3H), 4.38 (m, 6H), 6.75 (s, 1H), 6.80 (d, 2H), 7.25 (d, 1H), 7.50 (s, 1H), 7.80 (d, 2H), 7.90 (d, 2H), (LC/MS (M+H)⁺ m/z 560.3

237. 4-(4-bromo-1-{2-[2-chloro-4-(2-thienyl)phenoxy]ethyl}-5-ethoxy-1H-pyrazol-3-yl)benzamide: ¹H NMR (CDCl3): δ 1.41 (t, 3H), 4.45 (m, 6H), 6.90 (d, 1H), 7.05 (dd, 1H), 7.20 (dd, 2H), 7.40 (dd, 1H), 7.60 (s, 1H), 7.85 (d, 2H), 7.95 (d, 2H), (LC/MS (M+H)⁺ m/z 546.0

238. 4-(4-bromo-1-{2-[2-chloro-4-(2-thienyl)phenoxy]ethyl}-5-ethoxy-1H-pyrazol-3-yl)benzoic acid: ¹H NMR (Acetone): δ 1.41 (t, 3H), 4.45 (q, 2H), 4.58 (t, 2H), 4.65 (t, 2H), 7.10 (dd, 1H), 7.20 (d, 1H), 7.38 (dd, 2H), 7.55 (d, 1H), 7.65 (s, 1H), 8.03 (m, 4H), 11.20 (br.s, 1H), (LC/MS (M+H)⁺ m/z 547.5

239. methyl 4-(4-bromo-1-{2-[2-chloro-4-(2-thienyl)phenoxy]ethyl}-5-ethoxy-1H-pyrazol-3-yl)benzoate: ¹H NMR (CDCl₃): δ 1.41 (t, 3H), 3.92 (s, 3H), 4.45 (m, 6H), 6.90 (d, 1H), 7.05 (dd, 1H), 7.20 (m, 2H), 7.40 (m, 1H), 7.60 (m, 1H), 7.95 (d, 2H), 8.05 (d, 2H), (LC/MS (M+H)⁺ m/z 561.0

240. methyl 4-(4-bromo-1-{2-[2-chloro-4-(3-thienyl)phenoxy]ethyl}-5-ethoxy-1H-pyrazol-3-yl)benzoate: ¹H NMR (CDCl3): δ1.41 (t, 3H), 3.92 (s, 3H), 4.45 (m, 6H), 6.90 (d, 1H), 7.25 (dd, 1H), 7.30 (m, 3H), 7.60 (s, 1H), 7.95 (d, 2H), 8.05 (d, 2H), 11.20 (br. s, 1H), (LC/MS (M+H)⁺ m/z 561.0

241. 4-(4-bromo-1-{2-[2-chloro-4-(3-thienyl)phenoxy]ethyl}-5-ethoxy-1H-pyrazol-3-yl)benzoic acid: LC/MS MH+=547.5. RT=3.76 min

242. 4-(4-bromo-1-{2-[2-chloro-4-(3-thienyl)phenoxy]ethyl}-5-ethoxy-1H-pyrazol-3-yl)benzamide: LC/MS MH+=546.0 RT=3.55 min

243. methyl 4-{5-ethoxy-1-[2-(5,6,7,8-tetrahydro-1-naphthalenyloxy)ethyl]-1H-pyrazol-3-yl}benzoate: ¹H NMR (CD₂Cl₂, δ ppm) 8.04 (2H, d, J=6 Hz), 7.83 (2H, d, J=6 Hz), 7.03 (1H, t, J=6 Hz), 6.69 (1H, d, J=6 Hz), 6.63 (1H, d, J=6 Hz), 5.87 (1H, s), 4.41-4.43 (2H, m), 4.34-4.31 (2H, m), 4.18 (2H, t, J=6 Hz), 3.93 (3H, s), 2.71-2.73 (2H, m), 2.61-2.62 (2H, m), 1.76-1.72 (4H, m), 1.47 (3H, t). LC/MS (m+1) m/z=421.2

244. 4-{5-ethoxy-1-[2-(5,6,7,8-tetrahydro-1-naphthalenyloxy)ethyl]-1H-pyrazol-3-yl}benzoic acid: ¹H NMR (CD₃OD, δ ppm) 8.02 (2H, d, J=6 Hz), 7.83 (2H, d, J=6 Hz), 6.87 (1H, d, J=9 Hz), 6.61 (1H, d, J=3 Hz), 6.51 (1H, d, J=3 Hz), 6.12 (1H, s), 4.31-4.33 (3H, m), 4.20 (2H, q, J=9 Hz), 3.31-3.29 (2H, m), 2.65-2.64 (3H, m), 1.75-1.70 (4H, m), 1.40 (3H, t, J=9 Hz). LC/MS (m+1) m/z=407.2

245. 4-{5-ethoxy-1-[2-(5,6,7,8-tetrahydro-2-naphthalenyloxy)ethyl]-1H-pyrazol-3-yl}benzoic acid: ¹H NMR (CD₃OD, δ ppm) 8.05 (2H, d, J=6 Hz), 7.85-7.83 (2H, d, J=6 Hz), 6.89 (1H, d, J=6 Hz), 6.60 (1H, dd, J=6,3 Hz), 6.52 (1H, d, J=3 Hz), 6.12 (1H, s), 4.34-4.30 (4H, m), 4.20 (2H, q, J=6 Hz), 2.65-2.62 (4H, m), 1.75-1.70 (3H, m), 1.40 (3H, t, J=6 Hz). LC/MS (m+1) m/z=407.2

246. methyl 4-{1-[2-(2-chloro-4-methoxyphenoxy)ethyl]-5-ethoxy-1H-pyrazol-3-yl}benzoate: ¹H NMR (CD₂Cl₂, δ ppm) 8.05 (2H, d, J=9 Hz), 7.81 (2H, d, J=9 Hz), 6.91 (1H, d, J=3 Hz), 6.85-6.82 (1H, m), 6.73 (1H, dd, J=9, 3 Hz), 5.87 (1H, s), 4.42-4.37 (4H, m), 4.17 (2H, q, J=6 Hz), 3.92 (3H, s), 3.74 (3H, s), 1.45 (3H, t). LC/MS (m+1) m/z=431.3

247. 4-{1-[2-(2-chloro-4-methoxyphenoxy)ethyl]-5-ethoxy-1H-pyrazol-3-yl}benzoic acid: ¹H NMR (CD₃OD, δ ppm) 8.03 (2H, d, J=9 Hz), 7.85 (2H, d, J=9 Hz), 6.92 (2H, dd, J=9, 3 Hz), 6.76 (1H, dd, J=6, 3 Hz), 6.15 (1H, s), 4.39-4.36 (4H, m), 4.22 (2H, q, J=6 Hz), 3.30 (3H, s), 1.41 (3H, t, J=6 Hz). LC/MS (m+1) m/z=417.1

248. methyl 4-{1-[2-(2-chloro-4-methylphenoxy)ethyl]-5-ethoxy-1H-pyrazol-3-yl}benzoate: ¹H NMR (CD₂Cl₂, δ ppm) 8.05 (2H, d, J=9 Hz), 7.81 (2H, d, J=9 Hz), 6.91 (1H, d, J=3 Hz), 6.85-6.82 (1H, m), 6.73 (1H, dd, J=9, 3 Hz), 5.87 (1H, s), 4.42-4.37 (4H, m), 4.17 (2H, q, J=6 Hz), 3.92 (3H, s), 3.74 (3H, s), 1.45 (3H, t). LC/MS (m+1) m/z=415.3

249. 4-{1-[2-(2-chloro-4-methylphenoxy)ethyl]-5-ethoxy-1H-pyrazol-3-yl}benzoic acid: ¹H NMR (CD₃OD, δ ppm) 8.05 (2H, d, J=9 Hz), 7.81 (2H, d, J=9 Hz), 6.91 (1 H, d, J=3 Hz), 6.85-6.82 (1H, m), 6.73 (1H, dd, J=9 Hz, 3 Hz), 5.87 (1H, s), 4.42-4.37 (4H, m), 4.17 (2H, q, J=6 Hz), 3.74 (3H, s), 1.45 (3H, t). LC/MS (m+1) m/z=401.2

250. methyl 4-{1-[2-(2-chloro-4-ethylphenoxy)ethyl]-5-ethoxy-1H-pyrazol-3-yl}benzoate: ¹H NMR (CD₂Cl₂, δ ppm) 8.06 (2H, d, J=9 Hz), 7.83 (2H, d, J=9 Hz), 7.08 (2H, d, 6 Hz), 6.82 (2H, d, J=6 Hz), 5.87 (1H, s), 4.40-4.30 (4H, m), 4.18 (2H, q, J=9 Hz), 3.92 (3H, s), 2.57 (2H, q, J=6 Hz), 1.45 (3H, q, J=6 Hz), 1.19 (3H, J=6 Hz). LC/MS (m+1) m/z=395.4

251. 4-{1-[2-(2-chloro-4-ethylphenoxy)ethyl]-5-ethoxy-1H-pyrazol-3-yl}benzoic acid: ¹H NMR (CD₃OD, δ ppm) 8.06 (2H, d, J=9 Hz), 7.83 (2H, d, J=9 Hz), 7.08 (2H, d, J=6 Hz), 6.82 (2H, d, J=6 Hz), 5.87 (1H, s), 4.78-4.68 (2H, m), 4.42-4.36 (2H, m), 4.18 (2H, q, J=9 Hz), 2.57 (2H, q, J=6 Hz), 1.45 (3H, q, t=6 Hz), 1.19 (3H, t, J=6 Hz). LC/MS (m+1) m/z=381.2

252. methyl 4-{1-[2-(2-chloro-4-isopropylphenoxy)ethyl]-5-ethoxy-1H-pyrazol-3-yl}benzoate: ¹H NMR (CD₂Cl₂, δ ppm) 8.04 (2H, d, J=9 Hz), 7.82 (2H, d, J=9 Hz), 7.12 (2H, d, J=6 Hz), 6.82 (2H, d, J=6 Hz), 5.87 (1H, s), 4.40-4.34 (4H, m), 4.17 (2H, q, J=6 Hz), 3.92 (3H, s), 2.84 (1H, Septet, J=6 Hz), 1.44 (3H, t, J=6 Hz), 1.22 (3H, s), 1.20 (3H, s). LC/MS (m+1) m/z=409.4

253. 4-{1-[2-(2-chloro-4-isopropylphenoxy)ethyl]-5-ethoxy-1H-pyrazol-3-yl}benzoic acid: ¹H NMR (CD₃OD, δ ppm) 8.04 (2H, d, J=9 Hz), 7.82 (2H, d, J=9 Hz), 7.12 (2H, d, J=6 Hz), 6.82 (2H, d, J=6 Hz), 5.87 (1 H, s), 4.78-4.66 (2H, m), 4.42-4.38 (2H, m), 4.17 (2H, q, J=6 Hz), 2.84 (1H, Septet, J=6 Hz), 1.44 (3H, t, J=6 Hz), 1.22 (3H, s), 1.20 (3H, s). LC/MS (m+1) m/z=395.2

254. methyl 4-(1-{2-[(6-chloro-2,3-dihydro-1H-inden-5-yl)oxy]ethyl}-5-ethoxy-1H-pyrazol-3-yl)benzoate: ¹H NMR (CD₂Cl₂, δ ppm) 8.05 (2H, d, J=6 Hz), 7.85 (2H, d, J=6 Hz), 7.05 (1H, d,), 6.77 (1H, s), 6.67 (1H, dd, J=9, 3 Hz), 5.94 (1H, s), 4.37-4.34 (4H, s), 4.18 (2H, q, J=9 Hz), 3.91 (3H, s), 2.83 (4H, q, J=9 Hz), 2.03 (2H, t, J=6 Hz), 1.48 (3H, t, J=6 Hz). LC/MS (m+1) m/z=407.4

255. 4-(1-{2-[(6-chloro-2,3-dihydro-1H-inden-5-yl)oxy]ethyl}-5-ethoxy-1H-pyrazol-3-yl)benzoic acid: ¹H NMR (CD₃OD, δ ppm) 8.05 (2H, d, J=6 Hz), 7.85 (2H, d, J=6 Hz), 7.05 (1H, d,), 6.77 (1H, s), 6.67 (1H, dd, J=9, 3 Hz), 5.94 (1H, s), 4.79-4.66 (2H, m), 4.40-4.32 (2H, m), 4.18 (2H, q, J=9 Hz), 2.83 (4H, q, J=9 Hz), 2.03 (2H, t, J=6 Hz), 1.48 (3H, t, J=6 Hz). LC/MS (m+1) m/z=393.2

256. 4-{1-[2-(2-chloro-4-methoxyphenoxy)ethyl]-5-ethoxy-1H-pyrazol-3-yl}benzamide: ¹H NMR (CDCl₃ δ ppm) 7.74-7.76 (4H, m), 6.84 (1H, d, J=3 Hz), 6.75 (1H, d, J=9 Hz), 6.61 (1H, dd, J=9, 3 Hz), 5.79 (1H, s), 4.35-4.40 (4H, m), 4.12 (2H, q, J=6 Hz), 3.66 (3H, s), 1.36 (3H, t, J=9 Hz).

257. 4-{1-[2-(2-chloro-4-methylphenoxy)ethyl]-5-ethoxy-1H-pyrazol-3-yl}benzamide: ¹H NMR (CDCl₃, δ ppm) 7.82-8.84 (4H, m), 7.14 (1H, d, J=1.2 Hz), 6.93 (1H, d, J=9 Hz), 6.79 (1H, J=9 Hz), 5.86 (1H, s), 4.44-4.39 (4H, m), 4.16 (2H, q, J=9 Hz), 2.24 (3H, s), 1.45 (3H, t, J=6 Hz). LC/MS (m+1) m/z=400.3

258. methyl 3-(1-{2-[2-chloro-4-(4-methyl-2-thienyl)phenoxy]ethyl}-5-ethoxy-1H-pyrazol-3-yl)benzoate: ¹H NMR (CDCl3): δ 1.41 (t, 3H), 2.23 (s, 3H), 3.92 (s, 3H), 4.15 (q, 2H), 4.45 (br. s, 4H), 5.85 (s, 1H), 6.80 (s, 1H), 6.85 (d, 1H), 6.99 (s, 1H), 7.30 (dd, 1H), 7.42 (t, 1H), 7.55 (s, 1H), 7.90 (t, 2H), 8.38 (s, 1H), (LC/MS (M+H)⁺ m/z 497.2

259. 3-(1-{2-[2-chloro-4-(4-methyl-2-thienyl)phenoxy]ethyl}-5-ethoxy-1H-pyrazol-3-yl)benzoic acid: ¹H NMR (Acetone): δ 1.38 (t, 3H), 2.25 (s, 3H), 4.20 (s, 2H), 4.40 (t, 2H), 4.50 (t, 2H), 6.19 (s, 1H), 6.99 (s, 1H), 7.20 (d, 2H), 7.45 (m, 2H), 7.60 (s, 1H), 7.90 (d, 1H), 8.05 (d, 1H), 8.50 (s, 1H), 11.20 (br. s, 1H), (LC/MS (M+H)⁺ m/z 483.2

260. 1-[2-(2,4-dibromophenoxy)ethyl]-5-ethoxy-3-phenyl-1H-pyrazole:

261. 3-(1-{2-[2-chloro-4-(4-methyl-2-thienyl)phenoxy]ethyl}-5-ethoxy-1H-pyrazol-3-yl)-N-(4-morpholinylmethyl)benzamide: ¹H NMR (CDCl₃): 1.48 (t, 3H), 2.25 (s, 3H), 2.83-2.98 (br s, 2H), 3.30-3.39 (br s, 2H), 3.60-3.71 (m, 2H), 3.82-3.98 (m, 6H), 4.24 (q, 2H), 4.40-4.60 (m, 4H), 6.08 (s, 1H), 6.80 (s, 1H), 6.88 (d, 1H), 6.98 (s, 1H), 7.32 (d, 1H), 7.46 (t, 1H), 7.58 (s, 1H), 7.91 (t, 2H), 8.30 (s, 1H), 8.66 (S, 1H). HPLC-MS [M+1]⁺: m/z 595, RT 2.87

262. 3-(1-{2-[2-chloro-4-(4-methyl-2-thienyl)phenoxy]ethyl}-5-ethoxy-1H-pyrazol-3-yl)-N-[(diethylamino)methyl]benzamide: HPLC-MS [M+1]⁺: m/z 581, RT 2.89

263. 3-(1-{2-[2-chloro-4-(4-methyl-2-thienyl)phenoxy]ethyl}-5-ethoxy-1H-pyrazol-3-yl)-N-[(dimethylamino)methyl]benzamide: HPLC-MS [M+1]⁺: m/z 553, RT 2.83

264. 3-(1-{2-[2-chloro-4-(4-methyl-2-thienyl)phenoxy]ethyl}-5-ethoxy-1H-pyrazol-3-yl)-N-(2-methoxyethyl)benzamide: HPLC-MS [M+1]⁺: m/z 540, RT 3.73

265. 3-(1-{2-[2-chloro-4-(4-methyl-2-thienyl)phenoxy]ethyl}-5-ethoxy-1H-pyrazol-3-yl)-N-propylbenzamide: HPLC-MS [M+1]⁺: m/z 524, RT 3.82

266. 1-{2-[2-chloro-4-(2-furyl)phenoxy]ethyl}-3-phenyl-5-propoxy-1H-pyrazole: HPLC-MS [M+1]⁺: m/z 423, RT 3.97

267. 1-{2-[2-chloro-4-(2-thienyl)phenoxy]ethyl}-3-phenyl-5-propoxy-1H-pyrazole: HPLC-MS [M+1]⁺: m/z 439, RT 4.08

268. 1-{2-[2-chloro-4-(3-thienyl)phenoxy]ethyl}-3-phenyl-5-propoxy-1H-pyrazole: HPLC-MS [M+1]⁺: m/z 439, RT 4.03

269. 3-chloro-4-[2-(5-ethoxy-4-fluoro-3-phenyl-1H-pyrazol-1-yl)ethoxy]benzonitrile: LC/MS [M+1]⁺: m/z 386.3, RT 3.44

270. 4-[2-(4-bromo-5-ethoxy-3-phenyl-1H-pyrazol-1-yl)ethoxy]-3-chlorobenzonitrile: LC/MS [M+1]⁺: m/z 448.2, RT 3.54

271. 4-bromo-1-{2-[2-chloro-4-(trifluoromethyl)phenoxy]ethyl}-5-ethoxy-3-phenyl-1H-pyrazole: HPLC-MS [M+1]⁺: m/z 489, RT 4.18

272. 1-{2-[2-chloro-4-(trifluoromethyl)phenoxy]ethyl}-5-ethoxy-4-fluoro-3-phenyl-1H-pyrazole: HPLC-MS [M+1]⁺: m/z 429, RT 4.10

273. methyl 3-(1-{2-[2-chloro-4-(trifluoromethyl)phenoxy]ethyl}-5-ethoxy-1H-pyrazol-3-yl)benzoate: HPLC-MS [M+1]⁺: m/z 469, RT 3.87

274. methyl 3-{1-[2-(4-bromo-2-methylphenoxy)ethyl]-5-ethoxy-1H-pyrazol-3-yl}benzoate: HPLC-MS [M+1]⁺: m/z 459, RT 3.94

275. methyl 3-(1-{2-[2-chloro-4-(trifluoromethyl)phenoxy]ethyl}-5-ethoxy-4-fluoro-1H-pyrazol-3-yl)benzoate: HPLC-MS [M+1]⁺: m/z 487, RT 4.12

276. methyl 3-{1-[2-(4-bromo-2-methylphenoxy)ethyl]-5-ethoxy-4-fluoro-1H-pyrazol-3-yl}benzoate: HPLC-MS [M+1]⁺: m/z 477, RT 4.19

277. 1-[2-(4-bromo-2-methylphenoxy)ethyl]-5-ethoxy-4-fluoro-3-phenyl-1H-pyrazole: HPLC-MS [M+1]⁺: m/z 419, RT 4.19

278. 3-(1-{2-[2-chloro-4-(trifluoromethyl)phenoxy]ethyl}-5-ethoxy-4-fluoro-1H-pyrazol-3-yl)benzoic acid: HPLC-MS [M+1]⁺: m/z 473, RT 3.68

279. 3-{1-[2-(4-bromo-2-methylphenoxy)ethyl]-5-ethoxy-4-fluoro-1H-pyrazol-3-yl}benzoic acid: HPLC-MS [M+1]⁺: m/z 463, RT 3.67

280. 3-(1-{2-[2-chloro-4-(trifluoromethyl)phenoxy]ethyl}-5-ethoxy-1H-pyrazol-3-yl)benzoic acid: HPLC-MS [M+1]⁺: m/z 455, RT 3.51

281. 3-{1-[2-(4-bromo-2-methylphenoxy)ethyl]-5-ethoxy-1H-pyrazol-3-yl}benzoic acid: HPLC-MS [M+1]⁺: m/z 445, RT 3.54

282. methyl 3-{4-bromo-1-[2-(4-bromo-2-methylphenoxy)ethyl]-5-ethoxy-1H-pyrazol-3-yl}benzoate: HPLC-MS [M+1]⁺: m/z 540, RT 4.20

283. methyl 3-(4-bromo-1-{2-[2-chloro-4-(trifluoromethyl)phenoxy]ethyl}-5-ethoxy-1H-pyrazol-3-yl)benzoate: HPLC-MS [M+1]⁺: m/z 547, RT 4.24

284. 4-bromo-1-[2-(4-bromo-2-methylphenoxy)ethyl]-5-ethoxy-3-phenyl-1H-pyrazole: HPLC-MS [M+1]⁺: m/z 478, RT 4.20

285. 3-{4-bromo-1-[2-(4-bromo-2-methylphenoxy)ethyl]-5-ethoxy-1H-pyrazol-3-yl}benzoic acid: HPLC-MS [M+1]⁺: m/z 522, RT 3.77

286. 3-(4-bromo-1-{2-[2-chloro-4-(trifluoromethyl)phenoxy]ethyl}-5-ethoxy-1H-pyrazol-3-yl)benzoic acid: HPLC-MS [M+1]⁺: m/z 533, RT 3.67

287. 5-{3-chloro-4-[2-(5-ethoxy-3-phenyl-1H-pyrazol-1-yl)ethoxy]phenyl}-1,3-oxazole: TLC 50% Ethyl acetate/hexanes Rf 0.47, MS [M+] m/z 410

Method of Making the Compounds of the Present Invention

The particular process to be utilized in the preparation of the compounds of the present invention depends upon the specific compound desired. For example, such factors as the selection of the desired X and/or R moieties play a role in the path to be followed in the preparation of the specific compounds of this invention. Those factors are readily recognized by one of ordinary skill in the art.

In general, the compounds used in this invention may be prepared by standard techniques known in the art, by known processes analogous thereto, and/or by the processes described herein, using starting materials which are either commercially available or producible according to routine, conventional chemical methods.

General approaches to making the compounds of this invention can be found in “Advanced Organic Chemistry”, by J. March, John Wiley and Sons, 1985 and in “Comprehensive Organic Transformations”, by R. C. Larock, VCH Publishers, 1989, which are hereby incorporated by reference. In addition, many general preparations of pyrazole heterocycles of the present invention are known in the art (for example, Katritzky et al. in “Comprehensive Heterocyclic Chemistry II”, Elsevier Science Inc., 1996, incorporated herein by reference).

All references cited in this disclosure are incorporated herein by reference, in their entirety, for all purposes.

Even though the compounds of Formula I and Formula II may be prepared by use of a variety of known chemical reactions and procedures, the following general preparative methods are presented to aid the reader in synthesizing the compounds of the present invention, with more detailed particular examples being presented below. Compounds of Formula I and II are prepared as shown in Reaction Schemes 1 and 2, respectively.

A substituted naphthol (Scheme 1) or phenol (Scheme 2) may be converted to substituted pyrazolone compounds by first reacting the substituted naphthol or phenol with dibromoethylene in the presence of a base and a polar solvent such as NMP to form a bromoethyl ether. The ether is then coupled with hydrazine hydrate, in an alcohol such as ethanol, followed by cyclization with a substituted β-keto ester. The reaction produces a keto pyrazole as well as some of the corresponding alkoxy pyrazole of either Formula Ia (Formula I, where X=O) (Scheme 1) or Formula II (Scheme 2). The keto pyrazole product may also be O-alkylated with an alcohol of Formula R¹⁰OH under Mitsunobu conditions, or by reaction with a dialkylsulfide of Formula (R¹⁰)₂S and a base to produce the respective Formula Ia or II compounds. Formula Ib compounds (Formula I, where X═NH) may be prepared as shown in Scheme 1. Condensation of a cyanoketone with the hydrazine (III) gives a 3-amino pyrazole, (IV) that may be N-acylated to (V) and reduced to give the desired Ib compound.

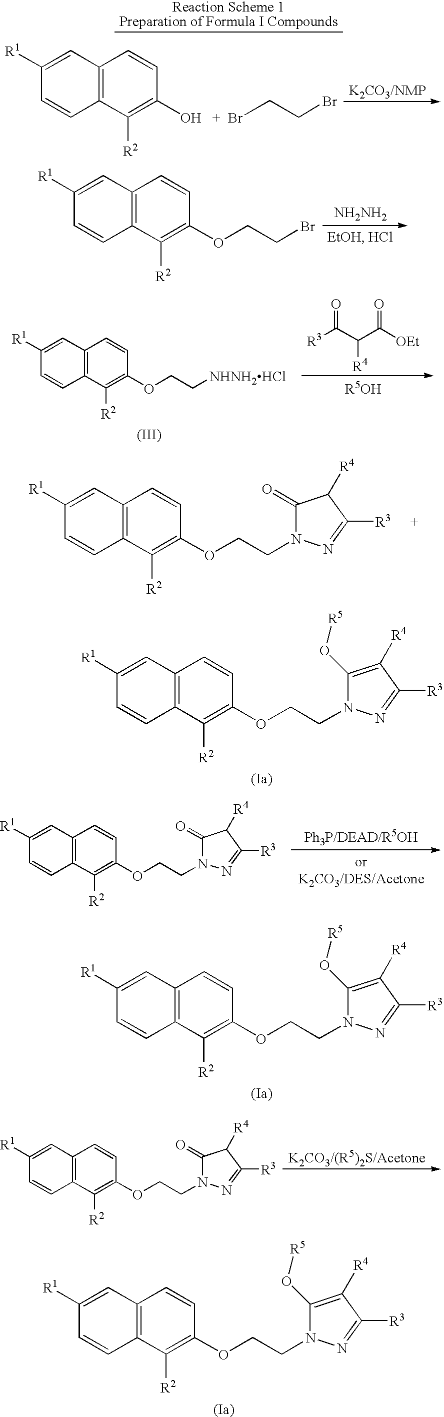

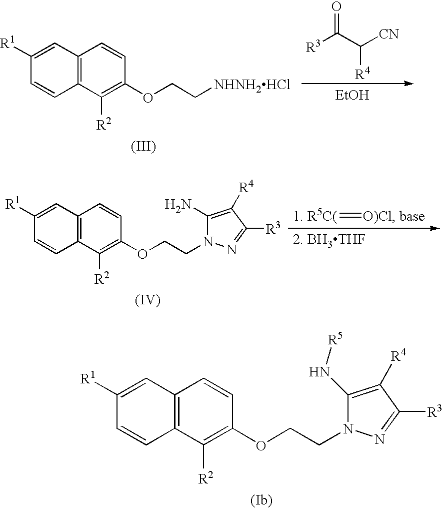

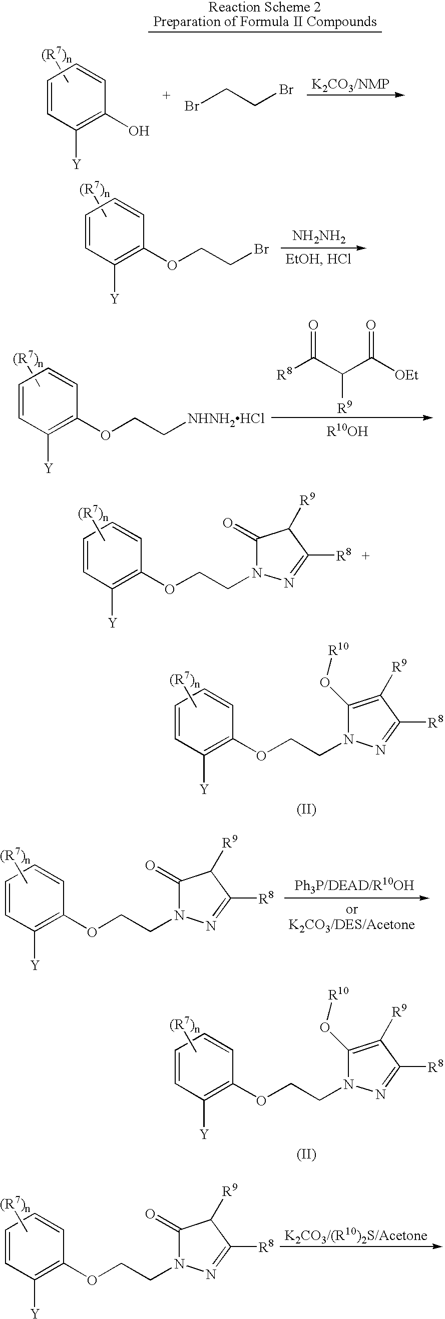

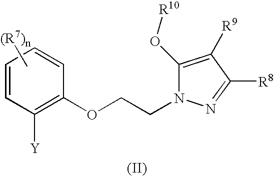

An alternative method for the preparation of Formula II compounds via substituted pyrazoles is shown in Reaction Scheme 3. 2-Hydroxyethylhydrazine is first allowed to react with a β-keto ester, then alkylated with a dialkylsulfide. Mitsunobu reaction of the hydroxyethyl pyrazole with a bromo-substituted phenol, followed by Suzuki coupling with an boronic acid of Formula R⁷B(OH)₂ gives compounds of Formula II.

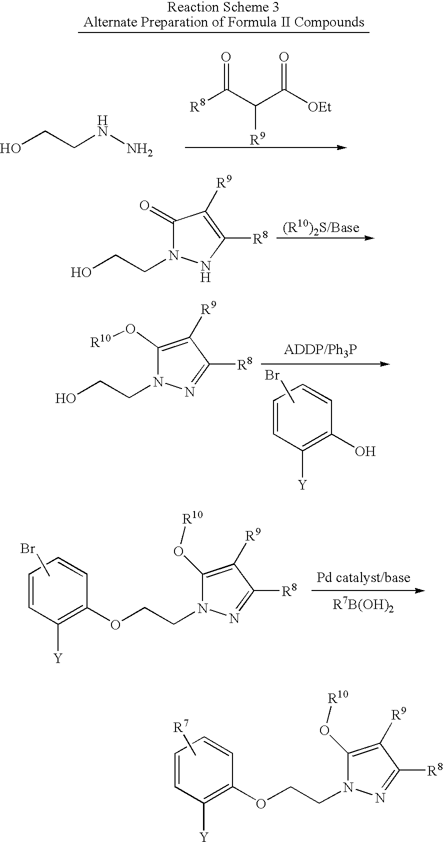

3-Unsubstituted pyrazoles can be converted to Formula II 3-halopyrazoles as shown in Scheme 4, using either NBS (bromo), NCS (chloro) or Selectofluor™(fluoro).

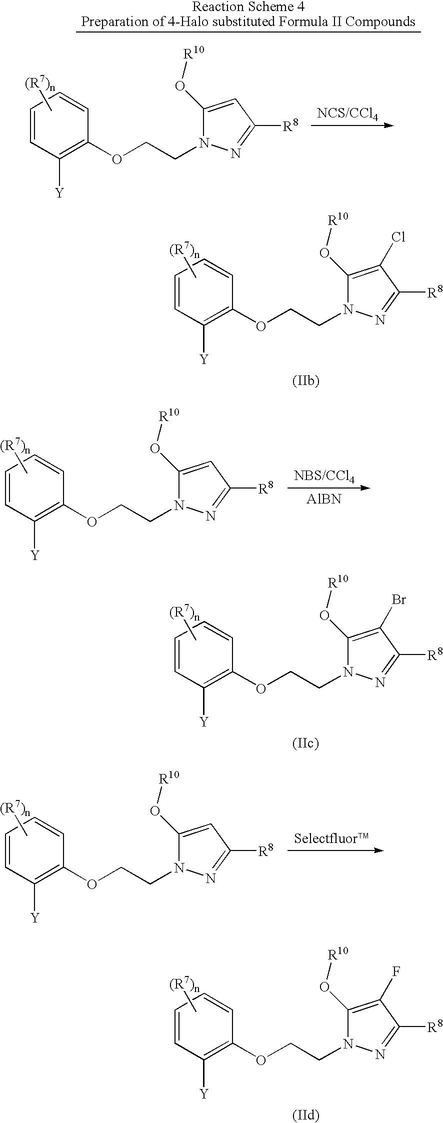

Specific examples of the manufacture of certain compounds of Formula I and Formula II are described below.

Abbreviations and Acronyms. When the following abbreviations are used throughout the disclosure, they have the following meaning:

CD₃OD methanol-d₄

CD₂Cl₂ methylene chloride-d₂

DCM dichloromethane

CH₃CN acetonitrile

DMF N,N-dimethylformamide

DES diethylsulphate

DPS dipropylsulphate

DMSO dimethylsulfoxide

EtOAc ethyl acetate

EtOH ethanol (100%)

Et₂O diethyl ether

Et₃N triethylamine

HEPES 2-[4-(2-hydroxyethyl)-1-piperazine]ethanesulfonic acid

HPLC high performance liquid chromatography

HPLC/MS high performance liquid chromatography/mass spectroscopy

LC/MS liquid chromatography/mass spectroscopy

MeOH methanol

MgSO₄ anhydrous magnesium sulfate

MPLC medium pressure liquid chromatography

MS/ES mass spectroscopy with electrospray

Na₂SO₄ anhydrous sodium sulfate

NH₄Cl ammonium chloride

RT Retention Time

THF tetrahydrofuran

TFA trifluoroacetic acid

TLC thin layer chromatography

All reactions were performed in oven-dried glassware under a positive pressure of dry argon, and were stirred magnetically unless otherwise indicated. Sensitive liquids and solutions were transferred via syringe or cannula, and introduced into reaction vessels through rubber septa. Commercial grade reagents and solvents were used without further purification. HPLC—electrospray mass spectra (HPLC ES-MS) were obtained using a Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 min at a flow rate of 1.0 mL/min was used with an initial hold of 0.5 min and a final hold at 95% B of 0.5 min. Total run time was 6.5 min.

### EXAMPLE 1

### 1-{2-[(1,6-dibromo-2-naphthyl)oxy]ethyl}-5-ethoxy-3-phenyl-1H-pyrazole

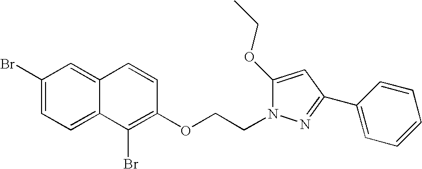

### Step 1: Preparation of 1,6-dibromo-2-(2-bromoethoxy)naphthalene

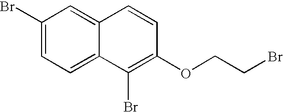

1,6 Dibromo-2-napthol (15. 0 g, 49.8 mmol) and potassium carbonate (19.0 g, 137.7 mmol) were dissolved in 1-methyl-2-pyroldinone (50 mL). The resulting mixture was stirred while being heated at 100° C. Dibromoethane (42.0 mL, 500 mmol) was added and the mixture was stirred at 100° C. for 72 h. After cooling to ambient temperature, water was added. The aqueous layer was extracted with diethyl ether (3×). The combined organic layers were washed with 1 N NaOH (3×), dried (Na₂SO₄), and evaporated. The crude product (19.0 g, 93%) was used for the next reaction without further purification.

### Step 2: Preparation of 1-{2-[(1,6-dibromo-2-naphthyl)oxy]ethyl}hydrazine hydrochloride

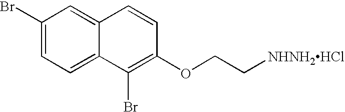

1,6-Dibromo-2-(2-bromoethoxy)naphthalene was dissolved in ethanol (50 mL) followed by addition of hydrazine monohydrate (24.3 mL, 500 mmol). The solution was heated at 80° C. for 16 h. After cooling to ambient temperature the reaction mixture was concentrated in vacuo. The residue was treated with 2N HCl (40 mL) and dichloromethane (20 mL) while stirring at room temp for 2 h. The precipitated yellow salt was filtered out and washed with water (2×) and dichloromethane (2×). Solid product (15.0 g, 78%) was dried under vacuum. LC/MS [M+1]⁺: m/z 361.0

### Step 3: Preparation of 2-{2-[(1,6-dibromo-2-naphthyl)oxy]ethyl}-5-phenyl-2,4-dihydro-3H-pyrazol-3-one

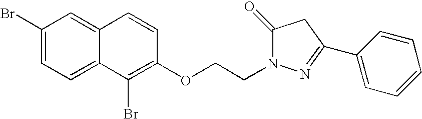

1-{2-[(1,6-Dibromo-2-naphthyl)oxy]ethyl}hydrazine hydrochloride (10.0 g, 25.9 mmol) was added to a solution of ethyl benzoylacetate (5.0 g, 4.5 mL, 25.9 mmol) in ethanol (50 mL). The resulting mixture was refluxed for 16 h. After cooling to ambient temperature, hydrochloric acid (2N, 10 mL) was added. The white precipitate formed was filtered and washed with water (2×). The solid was dried under vacuum and determined to be pyrazolone product 2-{2-[(1,6-dibromo-2-naphthyl)oxy]ethyl}-5-phenyl-2,4-dihydro-3H-pyrazol-3-one (5.0 g, 39%). ¹H-NMR (CDCl₃, 400 MHz) δ 3.62 (s, 1H), 4.25 (t, J=11.2 Hz, 2H), 4.48 (t, J=11.6 Hz, 2H), 7.16 (m, 1H), 7.25 (m, 1H), 7.38 (m, 2H), 7.55 (m, 1H), 7.62 (m, 3H), 7.89 (d, J=1.6 Hz, 1H), 8.02 (d, J=8.8 Hz, 1H). LC/MS [M+1]⁺: m/z 488.99

From the same reaction, 1-{2-[(1,6-dibromo-2-naphthyl)oxy]ethyl}-5-ethoxy-3-phenyl-1H-pyrazole (the step 4 product below) was also isolated as follows: The combined aqueous layers were extracted with ethyl acetate (3×), dried (Na₂SO₄), and concentrated in vacuo. Flash chromatography of the residue over silica gel with 5% EtOAc/DCM gave desired product (3.9 g, 29%). ¹H-NMR (CD₃OD, 400 MHz) δ 1.29 (m, 3H), 4.11 (m, 2H), 4.42 (m, 2H), 4.60 (m, 2H), 5.96 (s, 1H), 7.28 (m, 1H), 7.36 (m, 3H), 7.62 (m, 3H), 7.73 (m, 3H), 7.99 (d, J=2.0 Hz, 1H), 8.03 (d, J=9.2 Hz, 1H). LC/MS [M+1]⁺: m/z 517.04.

### Step 4: Preparation of the title compound: 1-{2-[(1,6-dibromo-2-naphthyl)oxy]ethyl}-5-ethoxy-3-phenyl-1H-pyrazole

A solution of 2-{2-[(1,6-dibromo-2-naphthyl)oxy]ethyl}-5-phenyl-2,4-dihydro-3H-pyrazol-3-one (0.890 g, 1.8 mmol), 1-1′-(azodicarbonyl) dipiperdine (0.919 g, 3.6 mmol), tributylphosphine (0.732 g, 0.896 mL, 3.6 mmol), and ethanol (1 mL) in toluene (10 mL) was stirred at 100° C. for 18 h. Then the reaction mixture was concentrated down in vacuo. Flash chromatography of the residue over silica gel with 5% EtOAc/DCM gave desired 1-{2-[(1,6-dibromo-2-naphthyl)oxy]ethyl}-5-ethoxy-3-phenyl-1H-pyrazole (0.730 g, 78%). ¹H-NMR (CD₃OD, 400 MHz) δ 1.29 (m, 3H), 4.11 (m, 2H), 4.42 (m, 2H), 4.60 (m, 2H), 5.96 (s, 1H), 7.28 (m, 1H), 7.36 (m, 3H), 7.62 (m, 3H), 7.73 (m, 3H), 7.99 (d, J=2.0 Hz, 1H), 8.03 (d, J=9.2 Hz, 1H). LC/MS [M+1]⁺: m/z 517.04.

Using procedures analogous to that described for Example 1, starting with the, appropriately substituted naphthols or phenols, and appropriate β-keto esters, the compounds of examples 2-34, 37-87 described in Table 1 were prepared.

### EXAMPLE 36

### 1-{2-[(1,6-dibromo-2-naphthyl)oxy]ethyl}-N-ethyl-3-phenyl-1H-pyrazol-5-amine

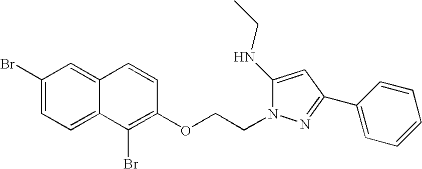

### Step 1: Preparation of 1-{2-[(1,6-dibromo-2-naphthyl)oxy]ethyl}-3-phenyl-1H-pyrazol-5-amine

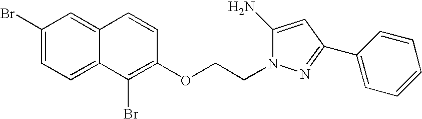

A solution of benzoylacetonitrile (500 mg, 3.45 mmol), and 1-{2-[(1,6-dibromo-2-naphthyl)oxy]ethyl}hydrazine hydrochloride (example 1, step 2) (1.365 g, 3.79 mmol) in EtOH (20.0 mL) was refluxed for 15 h. After removal of the solvents, flash chromatography of the residue over silica gel by using gradient solvents (20%, 40%, and 60% ethyl acetate in hexane) gave 1-{2-[(1,6-dibromo-2-naphthyl)oxy]ethyl}-3-phenyl-1H-pyrazol-5-amine as a yellow solid (230 mg, 15%). ¹H-NMR (CDCl₃, 400 MHz) δ 4.10-4.65 (m, 6H), 6.10 (s, 1H), 7.05-7.15 (m, 1H), 7.30-7.40 (m, 2H) 7.60-7.78 (m, 4H), 7.90 (s, 1H), 8.00 (d, 1H). LC/MS [M+1]⁺: m/z 488.02.

### Step 2: Preparation of N-(1-{2-[(1,6-dibromo-2-naphthyl)oxy]ethyl}-3-phenyl-1H-pyrazol-5-yl)acetamide

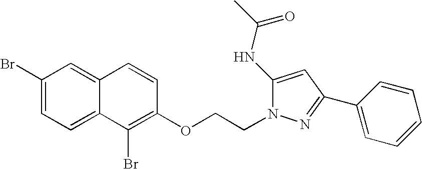

A mixture of compound 1-{2-[(1,6-dibromo-2-naphthyl)oxy]ethyl}-3-phenyl-1H-pyrazol-5-amine (100 mg, 0.21 mmol), acetyl chloride (38 μl, 0.41 mmol), and triethylamine (58 μl, 0.41 mmol) in a 1:1 mixture of DCE and NMP (4.0 mL) was stirred at ambient temperature for 72 h. Saturated NH₄Cl was added and the mixture was extracted with DCM (3×). The combined organic layers were dried and evaporated. Further purification using preparative TLC gave N-(1-{2-[(1,6-dibromo-2-naphthyl)oxy]ethyl}-3-phenyl-1H-pyrazol-5-yl)acetamide (80 mg) as a white solid.

### Step 3: Preparation of the title compound: 1-{2-[(1,6-dibromo-2-naphthyl)oxy]ethyl}-N-ethyl-3-phenyl-1H-pyrazol-5-amine

To a stirred solution of N-(1-{2-[(1,6-dibromo-2-naphthyl)oxy]ethyl}-3-phenyl-1H-pyrazol-5-yl)acetamide (40 mg, 0.07 mmol) in THF (0.75 mL) was added BH₃ (1.0 M in THF, 0.30 mL, 0.29 mmol). The resulting solution was heated at 50° C. for 6 h. After cooling to ambient temperature, MeOH (1.0 mL) was added and the resulting solution was refluxed overnight. Purification of the residue by preparative TLC using 5% acetone/DCM gave 1-{2-[(1,6-dibromo-2-naphthyl)oxy]ethyl}-N-ethyl-3-phenyl-1H-pyrazol-5-amine (28.8 mg, 80%). 1H-NMR (CDCl₃, 400 MHz) δ 1.25 (t, 3H), 3.14 (q, 2H), 4.40-4.62 (m, 4H), 5.75 (s, 1H), 7.05-7.15 (m, 1H), 7.30-7.40 (m, 2H), 7.60-7.78 (m, 4H), 7.90 (s, 1H), 8.00 (d, 1H). LC/MS [M+1]⁺: m/z 516.04.

Using procedure analogous to that described for Example 36, the compounds of examples 35 described in was prepared.

### EXAMPLE 70

### 1-{2[(3-chloro-4′-methoxy-1,1′-biphenyl-4-yl)oxy]ethyl}-5-ethoxy-3-phenyl-1H-pyrazole

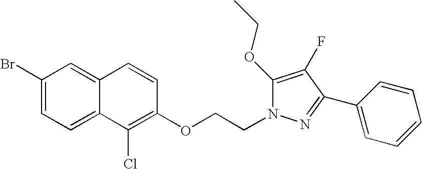

A solution of 1-{2-[(6-bromo-1-chloro-2-naphthyl)oxy]ethyl}-5-ethoxy-3-phenyl-1H-pyrazole (50 mg, 0.106 mmol) in anhydrous acetonenitrile (5 mL) was added 1-(chloromethyl)-4-fluoro-1,4-diazoniabicyclo[2,2,2]octane bis(tetrafluoroborate) (Selectfluor™) (45 mg, 0.127 mmol). The mixture was immediately cooled down to 0° C. After 2 hour stirring, at 0° C., TLC (EtOAc-hexane 1:4) showed a new fast moving spot appeared. The solution was added with 0.3 mL of NaHCO₃ aqueous and the solvent was concentrated. The residue was dissolved with minimum amount of CH₂Cl₂ and purified with Combiflash with 10 g silica gel. The column was eluted with 0 to 25% EtOAc in hexanes. After solvent was concentrated and dried under high vacuum to give 20 mg of the desired product. TLC Rf=1.7/4.3=0.40 (38% yield). LC/MS: MW+1 at m/z 489, RT 4.71 min. ¹H NMR (CD₂Cl₂) δ: 8.1 (m, 1H), 7.95 (d, 1H), 7.8 (m, 2H), 7.7 (m, 2H), 7.4 (m, 4H), 4.6 (t, 2H), 4.5 (t, 2H), 4.4 (2H), 1.4 (t, 3H).

Using procedures analogous to that described for Example 70, and using appropriately substituted pyrazoles, the compounds of examples 55-56, 58-59, 62-65, 70, 72-73, 75-76, 81, 131-134, 145,148, 165-188, 194-203, 207-232, 269, 272, 275-279 described in were prepared.

### EXAMPLE 88

### 1-{2-[(3-chloro-4′-methoxy-1,1′-biphenyl-4-yl)oxy]ethyl}-5-ethoxy-3-phenyl-1H-pyrazole

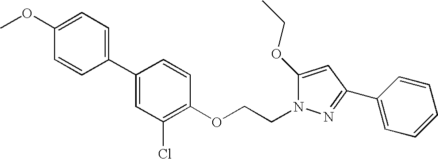

### Step 1: Preparation of 2-(2-hydroxyethyl)-5-phenyl-2,4-dihydro-3H-pyrazol-3-one

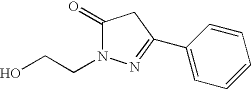

A mixture of ethyl benzoylacetate (50 mL, 0.26 mol) and 2-hydoxyethylhydrazine (20 mL, 0.29 mol) in 400 mL of toluene was heated at 125° C. (oil bath) with a Dean Stark overnight. The mixture was cooled down to room temperature, filtered, and treated with ether to form 41 g of desired product as a light brown precipitate (0.2 mol, 77% yield). ¹H NMR (DMSO): δ 3.85 (t, 2H), 4.1 (t, 2H), 5.90 (s, 1H), 7.35 (t, 1H), 7.5 (m, 2H), 7.8 (d, 2H); HPLC/MS (M+H)⁺ m/z 205.

### Step 2: 2-(5-ethoxy-3-phenyl-1H-pyrazol-1-yl)ethanol

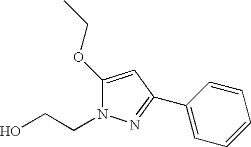

A mixture of 2-(2-hydroxyethyl)-5-phenyl-2,4-dihydro-3H-pyrazol-3-one (10 g, 0.049 mol), cesium carbonate (24 g, 0.073 mol), and diethyl sulfate (6.4 mL, 0.049 mol) in acetone (400 mL) was heated to reflux for 3 hrs. The solvent was filtered, and concentrated. The resulting residue was dissolved with CH₂Cl₂ and passed a short column eluted with CH₂Cl₂. After dried under vacuum at 40° C., 8.75 g of the desired product was isolated as a white precipitate (37.6 mmol, 77% yield). R_{f}=0.47 (EtOAc-hexanes, 1:1); HPLC/MS (M+H)⁺ m/z 233; ¹H NMR (CDCl₃): δ 1.45 (t, 3H), 4.0 (t, 2H), 4.1 (t, 2H), 4.2 (q, 2H), 5.85 (s, 1H), 7.30 (d, 1H), 7.35 (t, 2H), 7.75 (d, 2H).

### Step 3: 1-[2-(4-bromo-2-chlorophenoxy)ethyl]-5-ethoxy-3-phenyl-1H-pyrazole

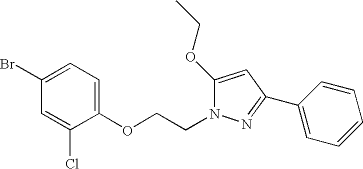

A mixture of 2-(5-ethoxy-3-phenyl-1H-pyrazol-1-yl)ethanol (0.40 g, 1.7 mmol), 4-bromo-2-chlorophenol (0.71 g, 3.4 mmol), ADDP (1,1′-(azodicarbonyl)dipiperidine) (0.87 g, 3.4 mmol), and tributylphosphine (0.86 mL) in toluene (20 mL) was heated at 115° C. (oil bath) overnight. The solvent was concentrated. The resulting residue was dissolved with CH₂Cl₂ and purified by a silica gel column chromatography eluting with EtOAc in hexanes (from 5 to 40%) to give 0.6 g of the desired product as a sticky oil (83% yield).

### Step 4: Preparation of the title compound: 1-{2-[(3-chloro-4′-methoxy-1,1′-biphenyl-4-yl)oxy]ethyl}-5-ethoxy-3-phenyl-1H-pyrazole

A mixture of 1-[2-(4-bromo-2-chlorophenoxy)ethyl]-5-ethoxy-3-phenyl-1H-pyrazole (35 mg, 0.083 mmol), 4-methoxyphenylboronic acid (23 mg, 0.11 mmol), Pd(dppf)Cl₂ (6 mg), and sodium bicarbonate (22 mg) in DME (2 mL) and water (0.4 mL) was heated at 115° C. overnight in a vial. The precipitate was filtered off, and the solvent was evaporated. The residue was purified with silica gel column eluted with EtOAc in hexanes (5-30%) to give 20 mg of 1-{2-[(3-chloro-4′-methoxy-1,1′-biphenyl-4-yl)oxy]ethyl}-5-ethoxy-3-phenyl-1H-pyrazole (54% yield). HPLC/MS: (M+H)⁺ m/z 449; R_{f}=0.34 (1:4, EtOAc/hexanes); ¹H NMR (CDCl₃): δ 1.45 (t, 3H), 3.85 (s, 3H), 4.2 (q, 2H), 4.45 (s, 4H), 5.8 (s, 1H), 6.95 (m, 3H), 7.3-7.45 (m, 6H), 7.52 (s, 1H), 7.75 (d, 2H).

Using procedures analogous to that described for Example 88, starting with the appropriate pyrazole, the compounds of examples 90-92 were prepared.

### EXAMPLE 89

### 1-[2-(4-bromo-2-chlorophenoxy)ethyl]-5-ethoxy-3-phenyl-1H-pyrazole

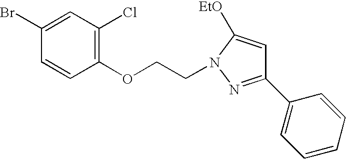

This compound was prepared using procedure analogous to example 1 shown above starting with 4-bromo-2-chlorophenol instead of 1,6 dibromo-2-napthol. TLC R_{f}=0.7 (3:7, EtOAc/hexanes); ¹H NMR (CDCl₃): δ 1.45 (t, 3H), 4.15 (q, 2H), 4.4 (d, 4H), 5.80 (s, 1H), 6.7 (d, 1H), 7.3-7.35 (m, 2H), 7.40 (t, 2H), 7.47 (s, 1H), 7.75 (d, 2H).

Using procedures analogous to that described for Example 89, and using appropriately substituted phenols, the compounds of examples 105-116, 119-124, 127-131,160, 172, 188, 191, 194-203, 225, 243-257, 260, 269-286 described in were prepared.

### EXAMPLE 104

### 1-{2-[2-chloro-4-(2-furyl)phenoxy]ethyl}-5-ethoxy-3-phenyl-1H-pyrazole

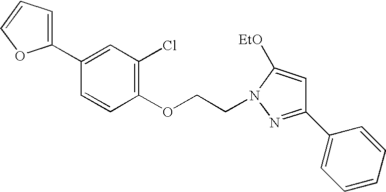

This compound was prepared using procedure analogous to example 88, step 4 starting with 1-[2-(4-bromo-2-chlorophenoxy)ethyl]-5-ethoxy-3-phenyl-1H-pyrazole (example 89) and 2-furanboronic acid. ¹H NMR (CDCl₃): δ 7.65 (t, 2H), 7.6 (d, 1H), 7.2-7.4 (m, 5H), 6.8 (t, 1H), 6.45 (d, 1H), 6.35 (d, 1H), 5.75 (d, 1H), 4.4 (d, 4H), 4.1 (q, 2H), 1.3 (t, 3H).

Using procedures analogous to that described for Example 104, the compounds of Examples 93-103, 118,125-126, 132-159, 161-171,173-187, 189-190,192-193, 204-224, 226-242, 258-259, 261-268 were prepared.

### EXAMPLE 117

### 1-{2-[2-chloro-4-(1H-pyrrol-1-yl)phenoxy]ethyl}-5-ethoxy-3-phenyl-1H-pyrazole

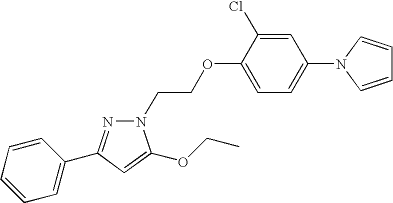

To a vial was added 1-[2-(4-bromo-2-chlorophenoxy)ethyl]-5-ethoxy-3-phenyl-1H-pyrazole (60 mg), pyrrole (20 uL), copper(I) trifluoromethanesulfonate benzene (5 mg), and 1,10-phenanthroline (26 mg), dba (trans, trans-dibenzylideneacetone) and cesium carbonate (51 mg) in m-xylene (anhyd, 1 mL). The mixture was heated to 120° C. on a heating block for 60 h. TLC showed that a slow moving spot had appeared, however, the starting pyrazole still could be observed. The mixture was purified by Combiflash (10 g silica gel), eluting with a gradient mixture of EtOAc in hexanes (5 to 15%). The solvent was removed and the residue was dried under high vacuum at 40° C. overnight to give 13 mg of a sticky oil (22% yield). TLC showed a single spot. ¹H NMR (CD₂Cl₂) showed a desired product and LC/MS showed m/z=408 (M+H).

### EXAMPLE 122

### 1-[2-(4-bromo-2-chlorophenoxy)ethyl]-5-ethoxy-3-phenyl-1H-pyrazole

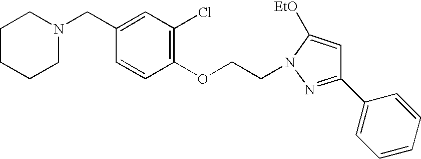

### Step 1: Preparation of 3-chloro-4-[2-(5-ethoxy-3-phenyl-1H-pyrazol-1-yl)ethoxy]benzaldehyde

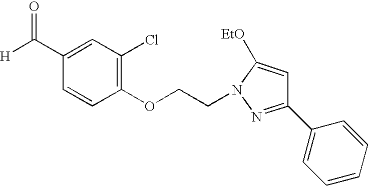

A mixture of 2-(5-ethoxy-3-phenyl-1H-pyrazol-1-yl)ethanol (example 88, step 2) (0.5 g), 3-chloro-4-hydroxybenzaldehyde (1.0 g), ADDP (1.63 g), and tributylphosphine (Bu₃P) (1.6 mL) in toluene (30 mL) was stirred at 100° C. over the weekend (84 hrs). The mixture was cooled down to room temperature and the insoluble material was filtered off, and washed with EtOAc. The solvent was concentrated and residue was dissovled with CH₂Cl₂ (16 mL) and purified with Combiflash (35 g of silica gel). The column was eluted with EtOAc in hexanes (form 10 to 60%, 50 min). TLC showed a poor separation. The mixture was combined and solvent was concentrated. The residue was dissolved with CH₂Cl₂ (5 mL) and purified with Combiflash again (35 g of silica gel) eluted with 5 to 30% EtOAc in hexanes to give 0.6 g (more pure) and 0.13 g (less pure) of product. However, ¹H NMR indicated both components contained impurities. TLC showed a more polar impurity very close the product. Rf=1.7/5.1=0.33 (EtOAc-hexanes 2:3). ¹H NMR (CD2Cl2): δ 9.74 (s, 1H), 7.65 (m, 3H), 7.3 (m, 2H), 7.2 (m, 1H), 6.95 (d, 1H), 5.75 (s, 1H), 4.45 (t, 2H), 4.35 (t, 2H), 4.05 (q, 2H), 1.35.(t, 3H).

### Step 2. Preparation of the title compound: 1-{3-chloro-4-[2-(5-ethoxy-3-phenyl-1H-pyrazol-1-yl)ethoxy]benzyl}piperidine:

To a vial was added sodium triacetoxyborohydride (85 mg), piperidine (26 uL), and 3-chloro-4-[2-(5-ethoxy-3-phenyl-1H-pyrazol-1-yl)ethoxy]benzaldehyde (80 mg) in 2 mL of 1,2-dichloroethane (anhydrous). The solution was stirred at room temperature for 80 hrs. TLC (CH2Cl2-MeOH-NH4OH, 9.5:0.5:0.025) showed a major slow moving spot appeared. Rf=1.8/4.7=0.33. No starting material A was observed. The solvent was filtered and washed with CH2Cl2. The mixture was purified with Combiflash (10 g silica gel), eluted with a mixed solution from 1 to 5% of MeOH (MeOH contained 5% of NH4OH aqueous ) in CH2Cl2. After the solvent was concentrated, co-evaporated with toluene, and dried under high vacuum at 40° C. over night, 40 mg of sticky oil was obtained (41% yield). TLC showed a single spot. LC/MS showed an mw+1 with m/z at 440, RT 2.01 min. 1H NMR (CD2Cl2): δ 7.7 (d, 2H), 7.3 (m, 2H), 7.2 (m, 2H), 7.05 (d, 1H), 6.8 (d, 1H), 5.75 (s, 1H), 4.35 (s, 4H), 4.05 (q, 2H), 3.25 (s, 2H), 2.2 (s, 4H), 1.45 (m, 4H), 1.3 (m, 5H).

Using procedures analogous to that described for Example 122, the compounds of examples 119-121 were prepared.

### EXAMPLE 137

### 4-(1-{2-[2-chloro-4-(2-thienyl)phenoxy]ethyl}-5-ethoxy-1H-pyrazol-3-yl)benzoic acid

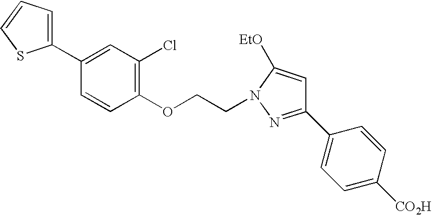

To a vial was added 1-[4-(2-thiophene)-2-chlorophenyl)ethyl]-5-ethoxy-3-[4-methoxycarbonyl)phenyl]-1H-pyrazole (50 mg, 0.104 mmol), and potassium hydroxide (21 mg, 0.52 mmol)in methanol (3 mL), water (1 mL), and THF (0.5 mL). The mixture was heated at 90° C. for 3 h. The solvent was evaporated and residue diluted with water and washed with hexanes. The aqueous layer was separated, and acidified slowly with 1 N HCl until pH=2. The solid precipitate was filtered off to give1-[4-(2-thiophene)-2-chlorophenyl)ethyl]-5-ethoxy-3-(4)-benzoic-1H-pyrazole, and dried under vac. oven at 40 C. overnight. The product was collected as a white solid (90%). TLC (Rf=0.07, EtOAc-hexanes 1:2); ¹H NMR (Acetone): δ 1.42 (t, 3H), 4.15 (q, 2H), 4.43 (t, 2H), 4.53 (t, 2H), 6.20 (s, 1H), 7.08 (dd, 1H), 7.18 (d, 1H), 7.39 (m, 2H), 7.54 (dd, 1H), 7.65 (d, 1H), 7.92 (d, 2H), 8.03 (d, 2H), 11.15 (br. s, 1H); LC/MS (M+H)⁺ m/z 469.4

Using procedures analogous to that described for Example 137, starting with the appropriate esters, the compounds of examples 22-23, 60-63, 66-67, 75-77, 80, 87, 136, 142-144, 146-147, 149, 153, 157-159, 168-171, 192, 232-233, 238, 241, 244-245, 247, 249, 251, 253, 255, 259, 278-281, 285-286 were prepared.

### EXAMPLE 221

### 1-[2-4-(2-thiophene)-2-chlorophenyl)ethyl]-5-ethoxy-4-fluoro-3-(4)-benzamide-1H-pyrazole

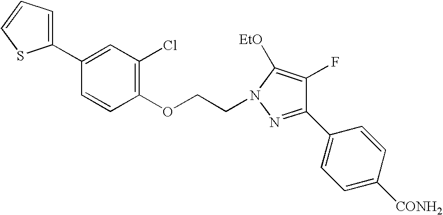

To a 10 mL reaction flask was added 1-[4-(2-thiophene)-2-chlorophenyl)ethyl]-5-ethoxy-4-fluoro-3-(4)-benzoic-1H-pyrazole (69 mg, 0.142 mmol), triethylamine (0.022 mL, 0.156 mmol), and ethyl chloroformate (0.015 mL, 0.142 mmol) in THF (4 mL) at 0° C. After stirring 30 min., NH4OH (0.017 mL, 0.426 mmol) was added and the mixture warmed to room temperature with stirring. The reaction mixture was diluted with EtOAc (5 mL), and washed with 0.5 N HCl (3×5 mL). The organic layer was separated, dried over Na2SO4, filtered, and concentrated to give 1-[24-(2-thiophene)-2-chlorophenyl)ethyl]-5-ethoxy-4-fluoro-3-(4)-benzamide-1H-pyrazole. The product was dried under vac. oven at 40 C. overnight, and collected as a white solid (87%). ¹H NMR (CDCl3): δ 1.41 (t, 3H), 4.40 (m, 6H), 6.85 (d, 1H), 7.05 (m, 1H), 7.20 (s, 1H), 7.25 (m, 2H), 7.38 (d, 2H), 7.60 (s, 1H), 7.80 (m, 4H), LC/MS (M+H)⁺ m/z 486.1

Using procedures analogous to that described for Example 221, starting with the appropriate carboxylic acids, the compounds of examples 64-65, 222-224, 236-237, 242, 256-257 were prepared.

### EXAMPLE 239

### 1-[4-(2-thiophene)-2-chlorophenyl)ethyl]-5-ethoxy-4-bromo-3-(4)-methoxycarbonyl-1-1H-pyrazole

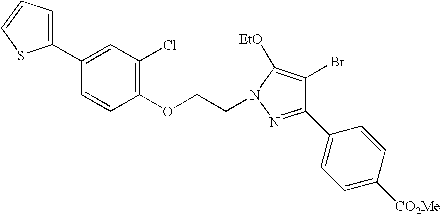

To a 10 mL reaction flask intially charged with CCl4 (5 mL) were added 1-[4-(2-thiophene)-2-chlorophenyl)ethyl]-5-ethoxy-3-(4)-methoxycarbonyl-1H-pyrazole (210 mg, 0.436 mmol), NBS (78 mg, 0.436 mmol), and AIBN (1 mg-catalyst). The mixture was stirred with heating to 60° C., for 3 h. Upon cooling to rt, the contents were concentrated in vacuo. The residue was washed with acetone, and solid product filtered off to furnish 1-[4-(2-thiophene)-2-chlorophenyl)ethyl]-5-ethoxy-4-bromo-3-(4)-methoxycarbonyl-1H-pyrazole. The product was dried under vac. oven at 40 C. overnight, and collected as a white solid (94%). ¹H NMR (CDCl3): δ 1.41 (t, 3H), 3.92 (s, 3H), 4.45 (m, 6H), 6.90 (d, 1H), 7.05 (dd, 1H), 7.20 (m, 2H), 7.40 (m, 1H), 7.60 (m, 1H), 7.95 (d, 2H), 8.05 (d, 2H), (LC/MS (M+H)⁺ m/z 561.0

Using procedures analogous to that described for Example 239, starting with the appropriate pyrazoles, 4-bromopyrazoles, as in the compounds of examples 54, 57, 60-61, 71, 74, 77, 79-80, 234-238, 240-242, 270-271, 282-286 were prepared.

### EXAMPLE 261

### 3-(1-{2-[2-chloro-4-(4-methyl-2-thienyl)phenoxy]ethyl}-5-ethoxy-1H-pyrazol-3-yl)-N-(4-morpholinylmethyl)benzamide

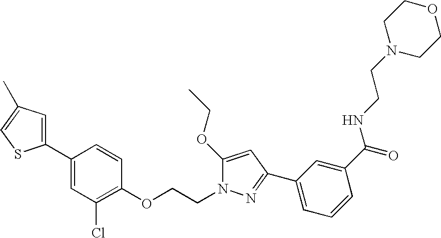

A reaction mixture containing example 259 (3-(1-{2-[2-chloro-4-(4-methyl-2-thienyl)phenoxy]ethyl}-5-ethoxy-1H-pyrazol-3-yl)benzoic acid, 50 mg, 0.1 mmol), 4-(2-aminoethyl)morpholine (13 mg, 0.1 mmol), EDCI (40 mg, 0.21 mmol), HOBT (28 mg, 0.21 mmol), and TEA (31 mg, 0.31 mmol) in methylene chloride was stirred at room temperature overnight. The reaction mixture was then concentrated under reduced pressure and the desired product was purifies by reverse phase HPLC. ¹H NMR (CDCl₃): 1.48 (t, 3H), 2.25 (s, 3H), 2.83-2.98 (br s, 2H), 3.30-3.39 (br s, 2H), 3.60-3.71 (m, 2H), 3.82-3.98 (m, 6H), 4.24 (q, 2H), 4.40-4.60 (m, 4H), 6.08 (s, 1H), 6.80 (s, 1H), 6.88 (d, 1H), 6.98 (s, 1H), 7.32 (d, 1H), 7.46 (t, 1H), 7.58 (s, 1H), 7.91 (t, 2H), 8.30 (s, 1H), 8.66 (S, 1H). HPLC-MS [M+1]⁺: m/z 595, RT 2.87

Using procedure analogous to that described for Example 261, starting with the example 259, examples 262-265 were prepared.

### EXAMPLE 287

### 5-{3-Chloro-4-[2-(5-ethoxy-3-phenyl-1H-pyrazol-1 -yl)ethoxy]phenyl}-1,3-oxazole

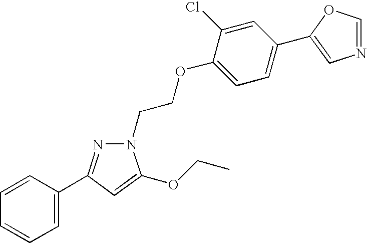

To a stirred suspension of K₂CO₃ (55 g) in anhydrous MeOH (10 mL) was added tosymethyl isocyanide. The solution was heated to 45° C. and 0.13 g of the aldehyde (from Step 1, Example 122) in 10 mL of anhydrous MeOH was added. The solution was heated to reflux for 2 h. TLC (EtOAc-hexanes, 1:1) showed the appearance of a slow moving spot, R_{f}=0.47. No starting material was observed. The solvent was concentrated, the residue was treated with CH₂Cl₂ and the insolubles were removed by filtration. The solvent was concentrated and purified with Combiflash (10 g silica gel), eluted with EtOAc in hexanes from 5 to 40%. After the solvent was concentrated and dried under high vacuum at 40° C. overnight, 15 mg of oil product was obtained (10% yield). LC/MS showed M+H m/z=410. ¹H NMR (CD₂Cl₂) confirmed the desired product.

Salts of the compounds identified herein can be obtained by isolating the compounds as hydrochloride salts, prepared by treatment of the free base with anhydrous HCl in a suitable solvent such as THF. Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. These methods are conventional and would be readily apparent to one skilled in the art.

Compositions of the Compounds of this Invention

The compounds of Formula I and Formula II can be utilized to achieve the desired pharmacological effect by administration to a patient in need thereof in an appropriately formulated pharmaceutical composition. A patient, for the purpose of this invention, is a mammal, including a human, in need of treatment (including prophylactic treatment) for the particular condition or disease. Therefore, the present invention includes pharmaceutical compositions which are comprised of a pharmaceutically acceptable carrier and a pharmaceutically effective amount of a compound, or salt thereof, of the present invention. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, otically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of 15 surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC-CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be illustrated as follows:

Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.

Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Method of Treating Hyper-proliferative Disorders

The present invention also relates to a method for using the compounds described of Formula I and Formula II to treat mammalian hyper-proliferative disorders. This method comprises administering to a mammal in need thereof, including a human, an amount of a compound of this invention, or a pharmaceutically acceptable salt thereof, which is effective to treat the disorder.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

The present invention also relates to a method for using the compounds of Formula I and Formula II as prophylactic or chemopreventive agents for prevention of the mammalian hyper-proliferative disorders described herein. This method comprises administering to a mammal in need thereof, including a human, an amount of a compound of this invention, or a pharmaceutically acceptable salt thereof, which is effective to delay or diminish the onset of the disorder.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The utility of the compounds of the present invention can be illustrated, for example, by their activity in vivo in the in vivo xenograft tumor model assay described below. The link between activity in tumor xenograft models in vivo and anti-tumor activity in the clinical setting is well established in the art (see, for example, Rose et al. Clin. Cancer Res 2001 July; 7(7):2016-21 and Goodman and Gilman's The Pharmacological Basis of Therapeutics (Ninth Edition), editor Molinoff et al., publ. by McGraw-Hill, pages 1225-1287, (1996)).

The following assay is one of the methods by which compound activity relating to prevention and/or treatment of the disorders identified herein can be determined.

In Vivo Tumor Model Assay

The tumor model selected for initial in vivo evaluation was an unstaged subcutaneous HCT-116 human colon tumor xenograft. Cells from HCT-116 tumor cell in vitro culture (5×10⁶ cells/animal) were implanted subcutaneously in the flank of mice. The mice were separated into a control group of 20 mice and three treatment groups of 10 mice each. Treatment was initiated 24 h later by the desired route and schedule. Test compounds were administered p.o. at dosages of 100 to 150 mg/kg/dose on a twice a day schedule (q7h×2) for 14 days (qd×14). Tumor growth and animal body weights were monitored twice per week. Efficacy was measured as the percent suppression of tumor progression relative to control. The mean size of the treated versus control tumors was monitored at each measurement and expressed as %T/C. Significance was evaluated by comparing the average tumor size in the treated and control groups at the end of treatment using a Student's t-test. Significance was set at p<0.05 for either test. Toxicity was assessed in terms of body weight loss and frank lethality was also recorded on a daily basis.

Control tumors grew in conformance to historical norms for this model. All treatments were well tolerated with no lethality and no weight loss in any group. Vehicle treatment had no significant effect on tumor growth. Compound tretament exhibited significant inhibition in tumor growth compared to vehicle treated tumors. Curves were statistically different from treated control (p=0.008) by two way ANOVA analysis. The T/C relative to the vehicle treated control at the end of treatment was not significantly different among the two doses evaluated.

Additionally, the compounds of this invention are useful in the prevention and/or treatment of, or in the manufacture of a medicament for treating, angiogenesis dependent disorders. A number of diseases are known to be associated with deregulated angiogenesis such as, for example, ocular neovascular disease, neovascular glaucoma, diabetic retinopathy, retrolental fibroplasia, hemangiomas, angiofibromas, psoriasis, age-related macula degeneration, haemangioblastoma, haemangioma, pain and inflammatory diseases such as rheumatoid or rheumatic inflammatory diseases including rheumatoid arthritis, as well as neoplastic diseases including, for example, so-called solid tumors and liquid tumors such as leukemias. As angiogenesis inhibitors, the compounds of this invention are also useful to control solid tumor growth such as breast, prostate, melanoma, renal, colon, cervical cancer, tumor metastasis, and the like.

Based upon the above and other standard laboratory techniques known to evaluate compounds useful for the prevention and/or treatment of the diseases or disorders described above by standard toxicity tests and by standard pharmacological assays for the determination of the prevention and/or treatment of the conditions identified above in mammals, and by comparison of these results with the results of known medicaments that are used to treat these conditions, the effective dosage of the compounds of this invention can readily be determined for prevention and/or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 200 mg/kg, and preferably from about 0.01 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound of the present invention or a pharmaceutically acceptable salt or ester or composition thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with known anti-hyper-proliferative or other indication agents, and the like, as well as with admixtures and combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone, irinotecan, raloxifen and topotecan.

The compounds or compositions of this invention can be administered as the sole pharmaceutical agent or in combination with one or more other pharmaceutical agents where the combination causes no unacceptable adverse effects. For example, the compounds of this invention can be combined with known anti-hyper-proliferative or other indication agents, and the like, as well as with admixtures and combinations thereof.

Optional anti-hyper-proliferative agents which can be added to or administered in conjunction with a compound or composition of this invention include but are not limited to compounds listed on the cancer chemotherapy drug regimens in the 11^{th} Edition of the Merck Index, (1996), which is hereby incorporated by reference. These compounds include asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of this invention either as a portion of a single composition containing more than one active ingredient, or as a separate drug to be administered in conjunction with a composition of this invention, include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine. Other anti-hyper-proliferative agents suitable for use with the composition of this invention include but are not limited to other anti-cancer agents such as epothilone, irinotecan, raloxifen and topotecan.

It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

Numerous modifications and variations in the invention as described in the above illustrative examples are expected to occur to those skilled in the art and consequently only those limitations as appear in the appended claims should be placed thereon. Accordingly it is intended in the appended claims cover all such equivalent variations which come within the scope of the invention as claimed

What is claimed is:

1. A compound of Formula I 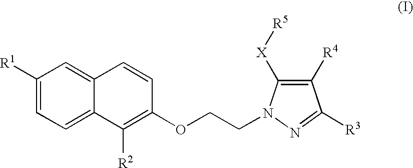
wherein
R¹ is H, halo or CN;
R² is H, CN, COR⁶, halo, or C₁-C₆alkyl;
R³ is CF₃,
C₁-C₆alkyl substituted with 0-1 substituent selected from
phenyl where the phenyl group is substituted with 0-5 substituents selected from C₁-C₆alkyl, C₁-C₆alkoxy, CF₃, NO₂, halo, CONH₂ and COOR⁶, and
phenoxy where the phenoxy group is substituted with 0-5 substituents selected from C₁-C₆alkyl,C₁-C₆alkoxy, CF₃, NO₂, halo, CONH₂ and COOR⁶, or
phenyl substituted with 0-5 substituents selected from C₁-C₆alkyl, C₁-C₆alkoxy, CF₃, NO₂, halo, CONH₂ and COOR⁶,
furyl substituted with 0-2 substituents selected from C₁-C₆alkyl and CF₃,
thienyl substituted with 0-2 substituents selected from halo and C₁-C₆alkoxy,
isoxazolyl substituted with 0-2 C₁-C₆alkyl substituents,
pyridyl, or
benzodioxole;
R⁴ is H, C₁-C₆alkyl, halo, or cyano;
X is O or NH;
R⁵ is C₁-C₆alkyl substituted with 0-1 substituent selected from CF₃, pyridyl, morpholinyl, and thienyl substituted with 0-1 C₁-C₆alkyl group;
R⁶ is H or C₁-C₆alkyl;
or a pharmaceutically acceptable salt thereof.

2. A compound of claim 1 wherein X is O.

3. A compound of claim 2 wherein R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

4. A compound of claim 3 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

5. A compound of claim 1 wherein X is NH.

6. A compound of claim 5 wherein R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

7. A compound of claim 6 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

8. A compound of Formula II 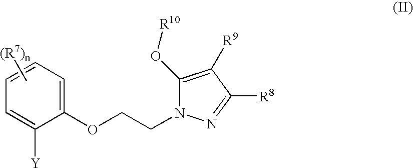
wherein
R⁷ is selected from C₁-C₆alkoxy, Br, Cl, F, CF₃, CN, COOH, NHCOR¹⁴,
C₁-C₆alkyl substituted with 0-1 substituent selected from COOH, NR¹²R¹², morpholine, pyrrolidine and piperidine,
phenyl substituted with from 0-3 substituents selected from C₁-C₆ alkyl, C₁-C₆alkoxy, SR¹⁴, Br, Cl, F, CF₃, NH₂ and phenyl,
a C₅-C₆ cyclic group,
thiophene substituted with 0-1 substituent selected from C₁-C₆alkyl and COR¹⁴,
pyridine with 0-2 substituents selected from Br, Cl, F, and C₁-C₆alkyl, pyrimidine substituted with 0-2 Br atoms, pyrrole, furan, oxazole, benzothiophene, benzofuran, morpholine, pyrrolidine, piperidine, naphthalene, and benzodioxole;
Y is H, C₁-C₆alkyl, C₁-C₆alkoxy, CN, Br, Cl, F, or I;
R⁸ is phenyl substituted with 0-2 substituents selected from C₁-C₆alkyl, C₁-C₆alkoxy, COR¹¹, and CONH(C₁-C₃alkyl)R¹¹;
R⁹ is H, C₁-C₆alkyl, Br, Cl, and F;
R¹⁰ is C₁-C₆alkyl substituted with 0-1 substituent selected from CF₃, pyridine, morpholine, and thiophene substituted with 0-1 C₁-C₆alkyl group;
R¹¹ is OH, NR¹²R¹², C₁-C₁₀alkyl, C₁-C₆alkoxy, C₁-C₆alkyl substituted with 0-1 substituent selected from CF₃ and morpholine;
R¹² is H and C₁-C₆alkyl;
R¹⁴ is C₁-C₆alkyl;.
n is 0, 1, or 2;
or a pharmaceutically acceptable salt thereof.

9. A compound of claim 8 wherein R⁷ is CF³, CN, C₁-C₆alkyl substituted with 0-1 substituent selected from COOH, and NR¹²R¹², COOH, phenyl substituted with 0-3 substituents selected from C₁-C₆alkyl, C₁-C₆alkoxy, CF₃, Br, Cl or F, or furan, or thiophene substituted with 0-2 substituents selected from halo and C₁-C₆alkoxy.

10. A compound of claim 9 wherein R⁷ is CF³, CN, furan or is thiophene substituted with 0-2 substituents selected from halo and C₁-C₆alkoxy, n is 0-1, R⁸ is phenyl substituted with 0-1 substituent selected from C₁-C₆alkyl, C₁-C₆alkoxy, and COOR¹¹, R¹¹ is OH, NR¹²R¹², C₁-C₁₀alkyl, and C₁-C₆alkoxy, and Y is Cl or C₁-C₆alkyl.

11. A pharmaceutical composition comprising a compound of Formula I 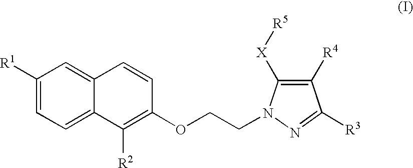
wherein
R¹ is H, halo or CN;
R² is H, CN, COR⁶, halo, or C₁-C₆alkyl;
R³ is CF₃,
C₁-C₆alkyl substituted with 0-1 substituent selected from
phenyl where the phenyl group is substituted with 0-5 substituents selected from C₁-C₆alkyl, C₁-C₆alkoxy, CF₃, NO₂, halo, CONH₂ and COOR⁶, and
phenoxy where the phenoxy group is substituted with 0-5 substituents selected from C₁-C₆alkyl,C₁-C₆alkoxy, CF₃, NO₂, halo, CONH₂ and COOR⁶,or
phenyl substituted with 0-5 substituents selected from C₁-C₆alkyl, C₁-C₆alkoxy, CF₃, NO₂, halo, CONH₂ and COOR⁶,
furyl substituted with 0-2 substituents selected from C₁-C₆alkyl and CF₃,
thienyl substituted with 0-2 substituents selected from halo and C₁-C₆alkoxy,
isoxazolyl substituted with 0-2 C₁-C₆alkyl substituents,
pyridyl, or
benzodioxole;
R⁴ is H, C₁-C₆alkyl, halo, or cyano;
X is O or NH;
R⁵ is C₁-C₆alkyl substituted with 0-1 substituent selected from CF₃, pyridyl, morpholinyl, and thienyl substituted with 0-1 C₁-C₆alkyl group;
R⁶ is H or C₁-C₆alkyl;
or a pharmaceutically acceptable salt thereof.

12. A pharmaceutical composition comprising a compound of Formula II 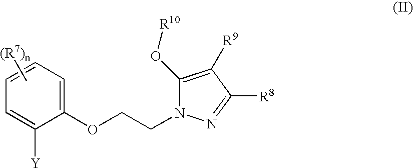
wherein
R⁷ is selected from C₁-C₆alkoxy, Br, Cl, F, CF₃, CN, COOH, NHCOR¹⁴,
C₁-C₆alkyl substituted with 0-1 substituent selected from COOH, NR¹²R¹², morpholine, pyrrolidine and piperidine,
phenyl substituted with from 0-3 substituents selected from C₁-C₆ alkyl, C₁-C₆alkoxy, SR¹⁴, Br, Cl, F, CF₃, NH₂ and phenyl,
a C₅-C₆ cyclic group, thiophene substituted with 0-1 substituent selected from C₁-C₆alkyl and COR¹⁴,
pyridine with 0-2 substituents selected from Br, Cl, F, and C₁-C₆alkyl,
pyrimidine substituted with 0-2 Br atoms,
pyrrole, furan, oxazole, benzothiophene, benzofuran, morpholine,
pyrrolidine, piperidine, naphthalene, and benzodioxole;
Y is H, C₁-C₆alkyl, C₁-C₆alkoxy, CN, Br, Cl, F, or I;
R⁸ is phenyl substituted with 0-2 substituents selected from C₁-C₆alkyl, C₁-C₆alkoxy, COR¹¹, and CONH(C₁-C₃alkyl)R¹¹;
R⁹ is H, C₁-C₆alkyl, Br, Cl, and F;
R¹⁰ is C₁-C₆alkyl substituted with 0-1 substituent selected from CF₃, pyridine, morpholine, and thiophene substituted with 0-1 C₁-C₆alkyl group;
R¹¹ is OH, NR¹²R¹², C₁-C₁₀alkyl, C₁-C₆alkoxy, C₁-C₆alkyl substituted with 0-1 substituent selected from CF₃ and morpholine;
R¹² is H and C₁-C₆alkyl;
R¹⁴ is C₁-C₆alkyl;
n is 0, 1, or 2;
or a pharmaceutically acceptable salt thereof.

13. A method of preventing and/or treating hyper-proliferative disorders in a mammal comprising administering to a mammal in need thereof a pharmaceutically effective amount of a compound of Formula I 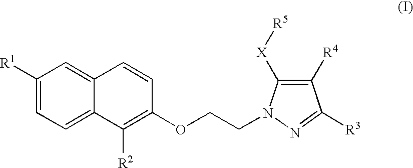
wherein
R¹ is H, halo or CN;
R² is H, CN, COR⁶, halo, or C₁-C₆alkyl;
R³ is CF₃,
C₁-C₆alkyl substituted with 0-1 substituent selected from
phenyl where the phenyl group is substituted with 0-5 substituents selected from C₁-C₆alkyl, C₁-C₆alkoxy, CF₃, NO₂, halo, CONH₂ and COOR⁶, and
phenoxy where the phenoxy group is substituted with 0-5 substituents selected from C₁-C₆alkyl,C₁-C₆alkoxy, CF₃, NO₂, halo, CONH₂ and COOR⁶,or
phenyl substituted with 0-5 substituents selected from C₁-C₆alkyl, C₁-C₆alkoxy, CF₃, NO₂, halo, CONH₂ and COOR⁶,
furyl substituted with 0-2 substituents selected from C₁-C₆alkyl and CF₃,
thienyl substituted with 0-2 substituents selected from halo and C₁-C₆alkoxy,
isoxazolyl substituted with 0-2 C₁-C₆alkyl substituents,
pyridyl, or
benzodioxole;
R⁴ is H, C₁-C₆alkyl, halo, or cyano;
X is O or NH;
R⁵ is C₁-C₆alkyl substituted with 0-1 substituent selected from CF₃, pyridyl, morpholinyl, and thienyl substituted with 0-1 C₁-C₆alkyl group;
R⁶ is H or C₁-C₆alkyl;
or a pharmaceutically acceptable salt thereof.

14. A method of claim 13 wherein X is O.

15. A method of claim 14 wherein R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

16. A method of claim 15 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

17. A method of claim 13 wherein X is NH.

18. A method of claim 17 R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

19. A method of claim 18 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

20. A method of preventing and/or treating hyper-proliferative disorders in a mammal comprising administering to a mammal in need thereof a pharmaceutically effective amount of a compound of Formula II 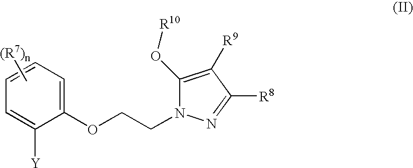
wherein
R⁷ is selected from C₁-C₆alkoxy, Br, Cl, F, CF₃, CN, COOH, NHCOR¹⁴,
C₁-C₆alkyl substituted with 0-1 substituent selected from COOH, NR²R¹², morpholine, pyrrolidine and piperidine,
phenyl substituted with from 0-3 substituents selected from C₁-C₆ alkyl, C₁-C₆alkoxy, SR¹⁴, Br, Cl, F, CF₃, NH₂ and phenyl,
a C₅-C₆ cyclic group,
thiophene substituted with 0-1 substituent selected from C₁-C₆alkyl and COR¹⁴,
pyridine with 0-2 substituents selected from Br, Cl, F, and C₁-C₆alkyl,
pyrimidine substituted with 0-2 Br atoms,
pyrrole, furan, oxazole, benzothiophene, benzofuran, morpholine,
pyrrolidine, piperidine, naphthalene, and benzodioxole;
Y is H, C₁-C₆alkyl, C₁-C₆alkoxy, CN, Br, Cl, F, or I;
R⁸ is phenyl substituted with 0-2 substituents selected from C₁-C₆alkyl, C₁-C₆alkoxy, COR¹¹, and CONH(C₁-C₃alkyl)R¹¹;
R⁹ is H, C₁-C₆alkyl, Br, Cl, and F;
R¹⁰ is C₁-C₆alkyl substituted with 0-1 substituent selected from CF₃, pyridine, morpholine, and thiophene substituted with 0-1 C₁-C₆alkyl group;
R¹¹ is OH, NR¹²R¹², C₁-C₁₀alkyl, C₁-C₆alkoxy, C₁-C₆alkyl substituted with 0-1 substituent selected from CF₃ and morpholine;
R¹² is H and C₁-C₆alkyl;
R¹⁴ is C₁-C₆alkyl;
n is 0, 1, or 2;
or a pharmaceutically acceptable salt thereof.

21. A method of claim 20 wherein R⁷ is CF³, CN, C₁-C₆alkyl substituted with 0-1 substituent selected from COOH, and NR¹²R¹², COOH, phenyl substituted with 0-3 substituents selected from C₁-C₆alkyl, C₁-C₆alkoxy, CF₃, Br, Cl or F, or furan, or thiophene substituted with 0-2 substituents selected from halo and C₁-C₆alkoxy.

22. A method of claim 21 wherein R⁷ is CF³, CN, furan or is thiophene substituted with 0-2 substituents selected from halo and C₁-C₆alkoxy, n is 0-1, R⁸ is phenyl substituted with 0-1 substituent selected from C₁-C₆alkyl, C₁-C₆alkoxy, and COOR¹¹, R¹¹ is OH, NR¹²R¹², C₁-C₁₀alkyl, and C₁-C₆alkoxy, and Y is Cl or C₁-C₆alkyl.

23. A method of preventing and/or treating angiogenesis dependent disorders comprising administering to a mammal in need thereof a pharmaceutically effective amount of a compound of Formula I 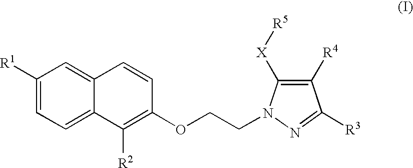
wherein
R¹ is H, halo or CN;
R² is H, CN, COR⁶, halo, or C₁-C₆alkyl;
R³ is CF₃,
C₁-C₆alkyl substituted with 0-1 substituent selected from
phenyl where the phenyl group is substituted with 0-5 substituents selected from C₁-C₆alkyl, C₁-C₆alkoxy, CF₃, NO₂, halo, CONH₂ and COOR⁶, and
phenoxy where the phenoxy group is substituted with 0-5 substituents selected from C₁-C₆alkyl,C₁-C₆alkoxy, CF₃, NO₂, halo, CONH₂ and COOR⁶, or
phenyl substituted with 0-5 substituents selected from C₁-C₆alkyl, C₁-C₆alkoxy, CF₃, NO₂, halo, CONH₂ and COOR⁶,
furyl substituted with 0-2 substituents selected from C₁-C₆alkyl and CF₃,
thienyl substituted with 0-2 substituents selected from halo and C₁-C₆alkoxy,
isoxazolyl substituted with 0-2 C₁-C₆alkyl substituents,
pyridyl, or
benzodioxole;
R⁴ is H, C₁-C₆alkyl, halo, or cyano;
X is O or NH;
R⁵ is C₁-C₆alkyl substituted with 0-1 substituent selected from CF₃, pyridyl, morpholinyl, and thienyl substituted with 0-1 C₁-C₆alkyl group;
R⁶ is H or C₁-C₆alkyl;
or a pharmaceutically acceptable salt thereof.

24. A method of claim 23 wherein X is O.

25. A method of claim 24 wherein R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

26. A method of claim 25 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

27. A method of claim 23 wherein X is NH.

28. A method of claim 27 wherein R³ is phenyl substituted with 0-5 substituents selected from C₁-C₆alkyl, C₁-C₆alkoxy, CF₃, NO₂, halo, CONH₂ and COOR⁶, or furyl substituted with 0-2 substituents selected from C₁-C₆alkyl and CF₃, or thienyl substituted with 0-2 substituents selected from halo and C₁-C₆alkoxy, or C₁-C₆alkyl substituted with phenyl where phenyl is substituted with 0-5 substituents selected from C₁-C₆alkyl, C₁-C₆alkoxy, CF₃, NO₂, halo, CONH₂ and COOR⁶.

29. A method of claim 28 wherein R¹ and R² are halo, R³ is phenyl or phenyl substituted with C₁-C₆alkyl or COOR⁶, R⁴ is halo, and R⁵ is C₁-C₆alkyl substituted with 0-1 CF₃ substituent.

30. A method of preventing and/or treating angiogenesis dependent disorders comprising administering to a mammal in need thereof a pharmaceutically effective amount of a compound of Formula II 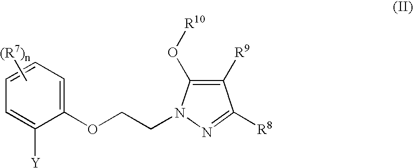
wherein
R⁷ is selected from C₁-C₆alkoxy, Br, Cl, F, CF₃, CN, COOH, NHCOR¹⁴,
C₁-C₆alkyl substituted with 0-1 substituent selected from COOH, NR¹²R¹², morpholine, pyrrolidine and piperidine,
phenyl substituted with from 0-3 substituents selected from C₁-C₆ alkyl, C₁-C₆alkoxy, SR¹⁴, Br, Cl, F, CF₃, NH₂ and phenyl,
a C₅-C₆ cyclic group,
thiophene substituted with 0-1 substituent selected from C₁-C₆alkyl and COR¹⁴,
pyridine with 0-2 substituents selected from Br, Cl, F, and C₁-C₆alkyl,
pyrimidine substituted with 0-2 Br atoms,
pyrrole, furan, oxazole, benzothiophene, benzofuran, morpholine, pyrrolidine, piperidine, naphthalene, and benzodioxole;
Y is H, C₁-C₆alkyl, C₁-C₆alkoxy, CN, Br, Cl, F, or I;
R⁸ is phenyl substituted with 0-2 substituents selected from C₁-C₆alkyl, C₁-C₆alkoxy, COR¹¹, and CONH(C₁-C₃alkyl)R¹¹;
R⁹ is H, C₁-C₆alkyl, Br, Cl, and F;
R¹⁰ is C₁-C₆alkyl substituted with 0-1 substituent selected from CF₃, pyridine, morpholine, and thiophene substituted with 0-1 C₁-C₆alkyl group;
R¹¹ is OH, NR¹²R¹², C₁-C₁₀alkyl, C₁-C₆alkoxy, C₁-C₆alkyl substituted with 0-1 substituent selected from CF₃ and morpholine;
R¹² is H and C₁-C₆alkyl;
R¹⁴ is C₁-C₆alkyl;
n is 0, 1, or 2;
or a pharmaceutically acceptable salt thereof.

31. A method of claim 30 wherein R⁷ is CF³, CN, C₁-C₆alkyl substituted with 0-1 substituent selected from COOH, and NR¹²R¹², COOH, phenyl substituted with 0-3 substituents selected from C₁-C₆alkyl, C₁-C₆alkoxy, CF₃, Br, Cl or F, or furan, or thiophene substituted with 0-2 substituents selected from halo and C₁-C₆alkoxy.

32. A method of claim 31 wherein R⁷ is CF³, CN, furan or is thiophene substituted with 0-2 substituents selected from halo and C₁-C₆alkoxy, n is 0-1, R⁸ is phenyl substituted with 0-1 substituent selected from C₁-C₆alkyl, C₁-C₆alkoxy, and COOR¹¹, R¹¹ is OH, NR¹²R¹², C₁-C₁₀alkyl, and C₁-C₆alkoxy, and Y is Cl or C₁-C₆alkyl.

33. A method of claim 13 where the hyper-proliferative disorder is selected from colon cancer, breast cancer and lung cancer.

34. A method of claim 33 where the disorder is colon cancer.
